(19) United States
(12) Patent Application Publication
(10) Pub. No.: US 2024/0043935 A1
(43) Pub. Date: Feb. 8, 2024

(54) EPIGENETICS ANALYSIS OF CELL-FREE DNA

(71) Applicant: Centre for Novostics Limited, New Territories (HK)

(72) Inventors: Yuk-Ming Dennis Lo, Kowloon (CN); Peiyong Jiang, New Territories (CN); Kwan Chee Chan, Kowloon (CN); Masashi Yukawa, New Territories (CN); Lu Ji, New Territories (CN); Jinyue Bai, New Territories (CN)

(21) Appl. No.: 18/227,831

(22) Filed: Jul. 28, 2023

Related U.S. Application Data
(60) Provisional application No. 63/393,725, filed on Jul. 29, 2022.

(51) Int. Cl. C12Q 1/6886; C12Q 1/6809; C12Q 1/6869; G16B 20/10; G16B 40/00

(52) U.S. Cl. CPC C12Q 1/6886; C12Q 1/6809; C12Q 1/6869; G16B 20/10; G16B 40/00; C12Q 2600/154; C12Q 2600/158

(57) ABSTRACT

Measuring quantities (e.g., relative frequencies) of particular sequence motifs of cell-free DNA fragments in a biological sample can be used to analyze the biological sample. The particular sequence motifs or sequence sizes in certain genomic regions may indicate a histone modification. The sequence motifs and/or sizes can be used to measure a property of the sample (e.g., fractional concentration of a tissue type or a characteristic of the tissue type), to measure an amount of histone modifications, to determine a condition of the organism based on such measurements, and to enrich a biological sample for clinically-relevant DNA. Different tissue types can exhibit different patterns for the relative frequencies of the sequence motifs. Measures of the relative frequencies of sequence motifs of cell-free DNA can be used for analysis.

### CROSS-REFERENCES TO RELATED APPLICATIONS

The present application claims priority to and is a non-provisional of U.S. Provisional Application No. 63/393,725, entitled “EPIGENETICS ANALYSIS OF CELL-FREE DNA,” filed on Jul. 29, 2022, the disclosure of which is incorporated by reference in its entirety for all purposes.

### BACKGROUND

Cell-free DNA (cfDNA) is a rich source of information that can be applied to the diagnosis and prognostication of many physiological and pathological conditions such as pregnancy and cancer (Chan, K. C. A. et al. (2017), New England Journal of Medicine 377, 513-522; Chiu, R. W. K. et al. (2008), Proceedings of the National Academy of Sciences of the United States of America 105, 20458-20463; Lo, Y. M. D. et al., (1997), The Lancet 350, 485-487). Cell-free DNA molecules in various bodily fluids (e.g., plasma, serum, urine, saliva, semen, peritoneal fluid, cerebrospinal fluid) may include a mixture of DNA molecules originating from various tissues. One mechanism whereby such cfDNA molecules are released is through cell death (e.g., apoptosis or necrosis). Selected cell populations, e.g., lymphocytes and neutrophils, have also been shown to secrete DNA molecules into bodily fluids. cfDNA molecules consist of fragmented DNA molecules. The correlation between cfDNA fragmentation patterns and nucleosome structures has been illustrated in many studies (Sun et al. Proc Natl Acad Sci USA. 2018; 115:E5106; Snyder et al. Cell. 2016; 164:57-68). Though circulating cfDNA is now commonly used as a non-invasive biomarker and is known to circulate in the form of short fragments, the physiological factors governing the fragmentation and molecular profile of cfDNA remain elusive.

Cell-free DNA may be analyzed to understand the epigenomic status. Epigenomic status of DNA may indicate regulation of genes, tissue origin, or diseases. The amount of histone modifications is an epigenomic factor. Conventional techniques to detect histone modifications involve using specific antibodies, relatively large amounts of sample, and more complicated sample handling. A simpler and more efficient technique is desired for determining epigenomic status of DNA. These and other needs are addressed.

### BRIEF SUMMARY

The present disclosure describes various techniques, such as measuring quantities (e.g., relative frequencies) of sequence motifs and sizes of cell-free DNA fragments in a biological sample of an organism for measuring a property of the sample (e.g., fractional concentration of a tissue type or a characteristic of the tissue type), measuring an amount of histone modifications, determining a condition of the organism based on such measurements, and enriching a biological sample for clinically-relevant DNA. Different tissue types exhibit different patterns for chromatin structures. The present disclosure provides various uses for deducing the chromatin structures based on the measures of the relative frequencies of sequence motifs and/or sizes of cell-free DNA, e.g., in mixtures of cell-free DNA from various tissues. DNA from one of a particular tissue may be referred to as clinically-relevant DNA.

Various examples can quantify amounts of sequence motifs representing the ending sequences of DNA fragments (i.e., end motifs). For example, embodiments can determine one or more relative frequencies of a set of one or more sequence motifs for ending sequences of DNA fragments. In various implementations, preferred sets of end motifs can be determined through using another technique (e.g., cfChIP-seq [cell-free Chromatin immunoprecipitation followed by sequencing) to measure an epigenomic status (e.g., histone modification) of chromatin in a particular region of a subject. The preferred sets of end motifs can be selected based on appearing more frequently in one or more regions with a particular epigenomic status compared to other end motifs. The particular epigenomic status can be associated with a particular tissue type or clinically-relevant DNA.

In various implementations, the relative frequencies of a preferred set can be used to measure a classification of a property (e.g., fractional concentration of clinically-relevant DNA) of a new sample, a condition (e.g., a gestational age of a fetus or a level of pathology) of the organism, or a measure of epigenomic status (e.g., histone modification amount). Accordingly, embodiments can provide measurements to inform physiological alterations, including cancers, autoimmune diseases, transplantation, and pregnancy.

As further examples, a preferred set of sequence end motif(s) can be used in a physical enrichment and/or an in silico enrichment of a biological sample for cell-free DNA fragments that are clinically-relevant. The enrichment can use sequence end motifs that are preferred for one or more genomic regions having particular histone modification(s). The particular histone modifications at the one or more genomic regions may be preferred for certain clinically-relevant tissue, such as fetal, tumor, or transplant. The physical enrichment can use one or more probe molecules that detect a particular set of sequence end motifs such that the biological sample is enriched for clinically-relevant DNA fragments. For the in silico enrichment, a group of sequence reads of cell-free DNA fragments having one of a set of preferred ending sequences for clinically-relevant DNA can be identified. Certain sequence reads can be stored based on a likelihood of corresponding to clinically-relevant DNA, where the likelihood accounts for the sequence reads including the preferred sequence end motifs. The stored sequence reads can be analyzed to determine a property of the clinically-relevant DNA the biological sample.

In some embodiments, the amount of DNA fragments in a certain size range can be used to determine the amount of a histone modification in cell-free DNA. The amount of histone modification deduced through the size information can be used to determine tissue fraction, a classification of a level of a disorder, and a status of a tissue or organ transplant.

Additionally, while a histone modification in a specific genomic region may indicate the DNA being of a specific type of tissue, histone modifications in many genomic regions may be the result of several different tissues. Using the histone modifications in genomic regions contributed by several different tissues may allow for more accurate analysis of a biological sample than using only histone modifications in genomic regions resulting from a single tissue. For example, using histone modifications contributed by several different tissues may result in more accurate analysis of the tissue origin and of the level of a disorder.

These and other embodiments of the disclosure are described in detail below. For example, other embodiments are directed to systems, devices, and computer readable media associated with methods described herein.

Reference to the remaining portions of the specification, including the drawings and claims, will realize other features and advantages of the present disclosure. Further features and advantages of the present disclosure, as well as the structure and operation of various embodiments of the present disclosure, are described in detail below with respect to the accompanying drawings. In the drawings, like reference numbers can indicate identical or functionally similar elements.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an illustration of the structure of DNA.

FIG. 2 shows using immunoprecipitation to analyze plasma cfDNA molecules associated with a histone modification.

FIG. 3 shows an illustration of the end motif of a fragment.

FIG. 4 is a graph defining categories of H3K4me3 regions with different levels of H3K4me3 ChIP signal according to embodiments of the present invention.

FIG. 5 is a table showing an example definition of categories of H3K4me3 regions using H3K4me3 ChIP-seq analysis of pregnant samples according to embodiments of the present invention.

FIG. 6 shows a table showing an example definition of categories of H3K27ac regions using H3K27ac ChIP-seq analysis of pregnant samples according to embodiments of the present invention.

FIG. 7 is a table showing an example definition of categories of H3K4me3 regions using H3K4me3 ChIP-seq analysis of samples from non-pregnant, healthy subjects according to embodiments of the present invention.

FIG. 8 is a table showing an example definition of categories of H3K27ac regions using H3K27ac ChIP-seq analysis of samples from non-pregnant, healthy subjects according to embodiments of the present invention.

FIG. 9 shows a heatmap of motif frequencies in regions with different levels of H3K4me3 ChIP signals for plasma DNA sequencing results, obviating a step of immunoprecipitation, according to embodiments of the present invention.

FIG. 10 is a graph of a comparison of end motif frequencies ranking between plasma DNA sequencing results with and without H3K4me3-based immunoprecipitation according to embodiments of the present invention.

FIG. 11 shows a table of 24 end motifs with the greatest ranking differences between conventional cfDNA sequencing and cfChIP-seq for H3K4me3 histone modification according to embodiments of the present invention.

FIG. 12A and FIG. 12B illustrate the use of end motif patterns to deduce plasma DNA histone modifications signal for plasma DNA sequencing results without immunoprecipitation according to embodiments of the present invention.

FIG. 13 shows a graph of the correlation between the aggregated abundance of end motifs overrepresented in H3K4me3-based immunoprecipitated plasma DNA and H3K4me3 ChIP signal according to embodiments of the present invention.

FIG. 14 is a graph showing the correlation between the cfChIP signal and the end motif frequency for 11 peak groups according to embodiments of the present invention.

FIGS. 15A and 15B are graphs showing the correlation between the cfChIP signal and the end motif frequency for six and eight peak groups according to embodiments of the present invention.

FIG. 16 is a graph of the correlation between the H3K4me3 ChIP signal in placenta-specific H3K4me3 regions deduced by end motifs and fetal DNA fraction determined by SNP-based approach according to embodiments of the present invention.

FIG. 17 is a flowchart of an example process associated with determining a fractional concentration of cell-free DNA fragments in a biological sample according to embodiments of the present invention.

FIG. 18 is a flowchart of an example process associated with estimating a first value of a characteristic of the target tissue according to embodiments of the present invention.

FIG. 19 is a flowchart of an example process associated with determining an amount of histone modification in one or more genomic regions using sequence motifs according to embodiments of the present invention.

FIG. 20 is a flowchart of an example process associated with determining an amount of histone modification in one or more genomic regions using fragmentomic features according to embodiments of the present invention.

FIG. 21 shows applying ChIP-seq (cell-free Chromatin immunoprecipitation followed by sequencing) to determine the contribution from different tissues according to embodiments of the present invention.

FIG. 22 is an ROC curve for differentiating patients with and without HCC using the deduced H3K4me3 signals in liver-specific H3K4me3 regions using end motifs according to embodiments of the present invention.

FIG. 23 is a flowchart of an example process associated with classifying a level of a disorder according to embodiments of the present invention.

FIGS. 24A, 24B, and 24C show percentages of cfDNA molecules with certain sizes in the region categories for different levels of H3K27ac signal according to embodiments of the present invention.

FIGS. 25A, 25B, and 25C show that the correlation between sizes and ChIP signals of histone modification can be generalized to other histone modifications according to embodiments of the present invention.

FIG. 26A and FIG. 26B illustrate the use of size information to deduce plasma DNA histone modifications for plasma DNA sequencing results without immunoprecipitation according to embodiments of the present invention.

FIGS. 27A, 27B, and 27C show the correlation between the percentage of cfDNA molecules within a size range and the log-transformed H3K4me3 ChIP signal according to embodiments of the present invention.

FIG. 28A shows evaluating the performance of deduced H3K4me3 ChIP signals in placenta-specific H3K4me3 regions for fetal DNA fraction deduction according to embodiments of the present invention.

FIG. 28B shows evaluating the performance of molecules within a certain size range in placenta-specific H3K4me3 regions for fetal DNA fraction deduction according to embodiments of the present invention.

FIG. 29 is a graph evaluating the performance of deduced H3K27ac ChIP signal in placenta-specific H3K27ac regions for determining fetal DNA fraction according to embodiments of the present invention.

FIG. 30 is a graph of the Pearson correlation coefficient of size ranges with and without calibration to the amount of H3K27ac signals in placenta-specific regions and fetal DNA fraction determined by SNP-based approach according to embodiments of the present invention.

FIG. 31A and FIG. 31B are graphs showing using deduced H3K4me3 ChIP signals based on liver-specific H3K4me3 regions for HCC detection according to embodiments of the present invention.

FIG. 32A and FIG. 32B show using deduced H3K27ac ChIP signals based on H3K27ac regions for HCC detection according to embodiments of the present invention.

FIG. 33 is a receiver operating characteristic (ROC) curve for differentiating subjects with intermediate and advanced stage hepatocellular carcinoma from healthy subjects according to embodiments of the present invention.

FIG. 34 is a graph showing correlation between deduced H3K27ac ChIP signals in liver-specific H3K27ac regions and donor DNA fraction according to embodiments of the present invention.

FIG. 35 is a graph of the Pearson correlation coefficient of size ranges with and without calibration to the amount of H3K27ac signals in the liver-specific regions and donor DNA fraction determined by SNP-based approach according to embodiments of the present invention.

FIG. 36 is a flowchart of an example process associated with determining an amount of histone modification in one or more genomic regions using fragment sizes according to embodiments of the present invention.

FIG. 37 shows a table of tissue-specific histone modification regions according to embodiments of the present invention.

FIG. 38 is a graph showing plasma DNA tissue mapping based on H3K4me3 histone modifications of cell-free DNA according to embodiments of the present invention.

FIG. 39 shows a graph showing the correlation between the placental contribution deduced by H3K4me3 ChIP signals according to embodiments of the present invention and the fetal DNA fraction according to embodiments of the present invention.

FIG. 40 is a graph of the contribution percentage of different tissues for both pregnant and non-pregnant samples based on H3K27ac histone modifications of cfDNA according to embodiments of the present invention.

FIG. 41 shows a heatmap of tissue contributions deduced from H3K27ac ChIP signal in pregnant and non-pregnant subjects according to embodiments of the present invention.

FIG. 42 is a graph of deduced H3K27ac ChIP signals across various tissue-specific region according to embodiments of the present invention.

FIG. 43A shows correlation between the placental contributions deduced by H3K27ac ChIP signals and the fetal DNA fraction determined by SNP-based approaches according to embodiments of the present invention.

FIG. 43B shows the correlation between normalized reads/kb in placental specific regions and the fetal DNA fraction determined by SNP-based approaches according to embodiments of the present invention.

FIG. 44 is an ROC curve for differentiating pregnant and non-pregnant subjects according to embodiments of the present invention.

FIG. 45 shows a receiver operating characteristic (ROC) curve for differentiating control subjects and subjects with colorectal cancer (CRC) using deduced colon contributions according to embodiments of the present invention.

FIG. 46A is a graph comparing erythroblast contributions deduced by H3K27ac ChIP signals between subjects with beta-thalassemia major and control subjects without beta-thalassemia major according to embodiments of the present invention.

FIG. 46B is an ROC curve for using the deduced erythroblast contribution to differentiate subjects with and without beta-thalassemia major according to embodiments of the present invention.

FIG. 47 is a heatmap of tissue contributions deduced using H3K27ac ChIP signals in subjects with beta thalassaemia major and control subjects according to embodiments of the present invention.

FIGS. 48A, 48B, and 48C show correlation between erythroid DNA percentage determined by ddPCR assay and the erythroblast contribution determined by H3K27ac signal according to embodiments of the present invention.

FIGS. 49A and 49B are graphs of deduced H3K27ac signals across healthy subjects, subjects with colorectal cancer (CRC) but without liver metastasis, and subjects with CRC and with liver metastasis according to embodiments of the present invention.

FIG. 50 is a graph of tissue contributions in urine and plasma DNA samples using H3K27ac histone modification of cell-free DNA according to the embodiments of the present invention.

FIG. 51 is a flowchart of an example process associated with determining a fractional concentration of a tissue type according to embodiments of the present invention.

FIG. 52 is a flowchart of an example process associated with determining a classification of a pregnancy or a disease according to embodiments of the present invention.

FIG. 53 illustrates input features in a machine learning model for determining a classification of a cancer according to embodiments of the present invention.

FIGS. 54A and 54B show results from a machine learning model in determining a classification of a cancer according to embodiments of the present invention.

FIG. 55 shows area under the curve (AUC) results for differentiating hepatocellular carcinoma (HCC) and non-HCC cases using machine learning models with different fragmentomic features according to embodiments of the present invention.

FIG. 56 is a flowchart of an example process associated with analyzing a biological sample of a subject to determine a classification of a condition of the subject according to embodiments of the present invention.

FIG. 57 is a flowchart of an example process associated with enriching a biological sample for clinically-relevant DNA according to embodiments of the present invention.

FIG. 58 is a flowchart of an example process associated with enriching a biological sample for clinically-relevant DNA according to embodiments of the present invention.

FIG. 59 illustrates a measurement system according to an embodiment of the present invention.

FIG. 60 shows a block diagram of an example computer system usable with systems and methods according to embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells.

A “biological sample” refers to any sample that is taken from a subject (e.g., a human (or other animal), such as a pregnant woman, a person with cancer, or a person suspected of having cancer, an organ transplant recipient or a subject suspected of having a disease process involving an organ (e.g., the heart in myocardial infarction, or the brain in stroke, or the hematopoietic system in anemia) and contains one or more nucleic acid molecule(s) of interest. The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), intraocular fluids (e.g., the aqueous humor), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 g×10 minutes, obtaining the fluid part, and re-centrifuging at for example, 16,000 g for another 10 minutes to remove residual cells. As part of an analysis of a biological sample, at least 1,000 cell-free DNA molecules can be analyzed. As other examples, at least 10,000 or 50,000 or 100,000 or 500,000 or 1,000,000 or 5,000,000 cell-free DNA molecules, or more, can be analyzed.

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

A “sequence read” refers to a string of nucleotides sequenced from any part or all of a nucleic acid molecule. For example, a sequence read may be a short string of nucleotides (e.g., nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification. As part of an analysis of a biological sample, at least 1,000 sequence reads can be analyzed. As other examples, at least 10,000 or 50,000 or 100,000 or 500,000 or 1,000,000 or 5,000,000 sequence reads, or more, can be analyzed.

A sequence read can include an “ending sequence” associated with an end of a fragment. The ending sequence can correspond to the outermost N bases of the fragment, e.g., 2-bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

A “sequence motif” of “sequence end signature” may refer to a short, recurring pattern of bases in nucleic acid fragments (e.g., cell-free DNA fragments). A sequence motif can occur at an end of a fragment, and thus be part of or include an ending sequence. An “end motif” can refer to a sequence motif for an ending sequence that preferentially occurs at ends of nucleic acid, e.g., DNA, fragments, potentially for a particular type of tissue. An end motif may also occur just before or just after ends of a fragment, thereby still corresponding to an ending sequence.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672).

A “relative frequency” (also referred to just as “frequency”) may refer to a proportion (e.g., a percentage, fraction, or concentration). In particular, a relative frequency of a particular end motif pair (e.g., A<>A) can provide a proportion of cell-free DNA fragments that have that particular pair of ending sequences.

An “aggregate value” may refer to a collective property, e.g., of relative frequencies of a set of end motifs. Examples include a mean, a median, a sum of relative frequencies, a variation among the relative frequencies (e.g., entropy, standard deviation (SD), the coefficient of variation (CV), interquartile range (IQR) or a certain percentile cutoff (e.g. 95^{th} or 99^{th} percentile) among different relative frequencies), or a difference (e.g., a distance) from a reference pattern of relative frequencies, as may be implemented in clustering. As another example, an aggregate value can comprise an array/vector of relative frequencies, which can be compared to a reference vector (e.g., representing a multidimensional data point).

A “calibration sample” can correspond to a biological sample whose fractional concentration of clinically-relevant nucleic acid (e.g., tissue-specific DNA fraction) is known or determined via a calibration method, e.g., using an allele specific to the tissue, such as in transplantation whereby an allele present in the donor's genome but absent in the recipient's genome can be used as a marker for the transplanted organ. As another example, a calibration sample can correspond to a sample from which end motifs can be determined. A calibration sample can be used for both purposes. Multiple calibration samples may be used As an example, a first calibration sample can correspond to a biological sample, which has measurable histone modification levels across various genomic regions of interest. A second calibration sample can correspond to a biological sample, which has measurable fragmentomic features across various genomic regions of interest. The first and second calibration samples can be used together for determining the calibration values.

A “calibration data point” includes a “calibration value” and a measured or known characteristic value of a target tissue type or a fractional concentration of the clinically-relevant nucleic acid (e.g., DNA of particular tissue type). The calibration value can be determined from various types of data measured from nucleic acid molecules of a sample, e.g., amounts of end motifs or fragment sizes. The calibration value corresponds to a parameter that correlates to the desired property, e.g., characteristic value of a target tissue type or a fractional concentration of the clinically-relevant DNA. For example, a calibration value can be determined from relative frequencies (e.g., an aggregate value) of end signatures as determined for a calibration sample, for which the desired property is known. The calibration data points may be defined in a variety of ways, e.g., as discrete points or as a calibration function (also called a calibration curve or calibration surface). The calibration function could be derived from additional mathematical transformation of the calibration data points. In some embodiments, a “calibration data point” may include a “calibration value” and a measured or known characteristic values (e.g., fragmentomic features) of a group of genomic regions of interest (e.g., characterized by certain levels of histone modifications).

A “separation value” corresponds to a difference or a ratio involving two values, e.g., two fractional contributions or two methylation levels. The separation value could be a simple difference or ratio. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (ln) of the two values. A separation value can include a difference and a ratio.

A “separation value” and an “aggregate value” (e.g., of relative frequencies) are two examples of a parameter (also called a metric) that provides a measure of a sample that varies between different classifications (states), and thus can be used to determine different classifications. An aggregate value can be a separation value, e.g., when a difference is taken between a set of relative frequencies of a sample and a reference set of relative frequencies, as may be done in clustering.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1). As further examples, the levels of classification can correspond to a fractional concentration or a value for a characteristic, e.g., of a sample or of a target tissue type.

The term “parameter” as used herein means a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter.

The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. Such a reference value can be determined in various ways, as will be appreciated by the skilled person. For example, metrics (parameters) can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity). A parameter can be compared to cutoff value, threshold value, reference value, or calibration value to determine a classification Such a process for determining such values can be performed as part of training a machine learning model, e.g., which receives a training vector of a set of one or more parameters. And the comparison of a parameter(s) to any of such values can be accomplished by inputting the parameter(s) into a machine learning model, e.g., that was trained that was trained using the parameter values determined from other subjects, e.g., ones with or without a condition, abnormality, or pathology or ones with a known parameter values (e.g., a calibration value).

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer. A “level of disease” is similar to “level of cancer” but can refer to a disease rather than cancer.

A “level of abnormality” can refer to the amount, degree, or severity of abnormality associated with an organism, where the level can be as described above for cancer. An example of abnormality is pathology associated with the organism. Another example of abnormality is a rejection of a transplanted organ. Other example abnormalities can include autoimmune attack (e.g., lupus nephritis damaging the kidney or multiple sclerosis), inflammatory diseases (e.g., hepatitis), fibrotic processes (e.g., cirrhosis), fatty infiltration (e.g., fatty liver diseases), degenerative processes (e.g., Alzheimer's disease) and ischemic tissue damage (e.g., myocardial infarction or stroke). A heathy state of a subject can be considered a classification of normal.

The term “gestational age” can refer to a measure of the age of a pregnancy which is taken from the beginning of the woman's last menstrual period (LMP), or the corresponding age of the gestation as estimated by a more accurate method if available. Such methods include adding 14 days to a known duration since fertilization (as is possible in in vitro fertilization), or by obstetric ultrasonography.

A “pregnancy-associated disorder” includes any disorder characterized by abnormal relative expression levels of genes in maternal and/or fetal tissue or by abnormal clinical characteristics in the mother and/or fetus. These disorders include, but are not limited to, preeclampsia (Kaartokallio et al. Sci Rep. 2015; 5:14107; Medina-Bastidas et al. Int J Mol Sci. 2020; 21:3597), intrauterine growth restriction (Faxén et al. Am J Perinatol. 1998; 15:9-13; Medina-Bastidas et al. Int J Mol Sci. 2020; 21:3597), invasive placentation, pre-term birth (Enquobahrie et al. BMC Pregnancy Childbirth. 2009; 9:56), hemolytic disease of the newborn, placental insufficiency (Kelly et al. Endocrinology. 2017; 158:743-755), hydrops fetalis (Magor et al. Blood. 2015; 125:2405-17), fetal malformation (Slonim et al. Proc Natl Acad Sci USA. 2009; 106:9425-9), HELLP syndrome (Dijk et al. J Clin Invest. 2012; 122:4003-4011), systemic lupus erythematosus (Hong et al. J Exp Med. 2019; 216:1154-1169), and other immunological diseases of the mother.

A “site” (also called a “genomic site”) corresponds to a single site, which may be a single base position or a group of correlated base positions, e.g., a CpG site or larger group of correlated base positions. A “locus” may correspond to a region that includes multiple sites. A locus can include just one site, which would make the locus equivalent to a site in that context.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and in some versions within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. It is also to be understood that the endpoints of the range provided are included in the range. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described

### DETAILED DESCRIPTION

Epigenomic status of different regions of chromatin (DNA and proteins) may indicate the expression activities of genes, tissue origin, or diseases. A histone modification is an example of an epigenomic factor where measurements of the amount of histones having a particular epigenomic status can be used in various ways. Techniques to detect histone modifications include cfChIP-seq (cell-free Chromatin immunoprecipitation followed by sequencing), which has some disadvantages. The cfChIP-seq technique requires 1-2 ml or more of sample, which is a large sample compared to the hundreds of microliters or less used when just sequencing is performed. In addition, cfChIP-seq uses more complicated and time-consuming sample techniques, compared with procedures of conventional plasma cfDNA-seq. In the cfChIP-seq procedure, the target epigenome is linked to proteins (e.g., histone modification). Proteins are unstable compared to DNA. Freeze, thaw, and storage conditions affect the stability of protein more than that of DNA.

This disclosure shows that certain end motifs of cell-free DNA (i.e., sequences at ends of the naturally fragmented DNA), sizes, and/or other fragmentomic features are highly correlated with histone modifications. The amount of these end motifs can indicate the amount of a histone modification in a sample, and therefore a subject. As a result, the end motifs can be used to indicate the activity of genes, tissue origin, or disease, avoiding the disadvantages of cfChIP-seq. Analyzing end motifs can use sequencing techniques that do not require the extra steps of cfChIP-seq. As a result, embodiments of the present invention can use less than 100 μl of biological sample, which can include about 500 pg of cell-free DNA. Sampling handling for sequencing is much simpler than with cfChIP-seq techniques. Samples do not need to be frozen to temperatures of less than −80° C. Samples can be shipped farther distances from a clinic to a laboratory. In addition, analyzing end motifs can be applied to study multiple, different epigenome types from a single measurement, rather than limited by the specific histone modification tied to the specific antibody used in a particular cfChIP-seq assay.

Measuring certain end motifs of cell-free DNA can therefore provide an improved technique of determining an epigenomic status of a particular region of chromatin, e.g., corresponding to a particular region of a reference genome. Additionally, measuring certain end motifs can also determine different properties of a sample where such a property is associated with the epigenomic status of the particular region, such as fractional concentration of a tissue type, classification of a disorder, gestational age, nutrition status of an organ, size of an organ, or other properties. These properties may be determined using the epigenomic status determined from the end motifs or directly from the end motifs.

Samples can be physically or in silico enriched for certain end motifs that are more frequently associate with certain epigenomic statuses, including histone modifications. Enrichment of samples may allow for more accurate measurements of a property of a sample, measuring an amount of histone modification, or determining a condition of an organism.

### I. Epigenomic Status

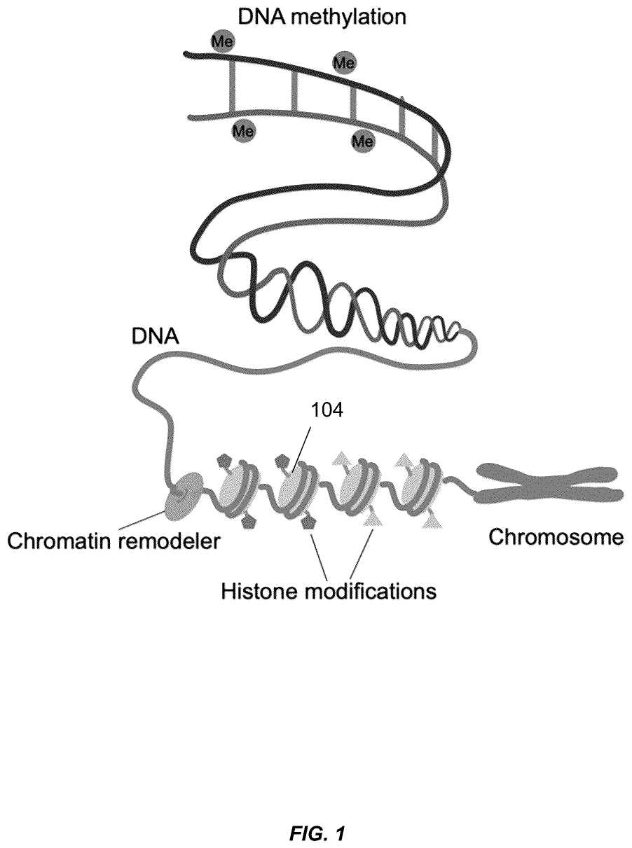

FIG. 1 shows an illustration of the structure of DNA. DNA within a cell is a large structure. Besides the nucleotides in DNA, DNA is lumped with several different proteins, including chromatin remodeler, transcription factors, nucleosome, and histones. Histones are proteins that the DNA winds around. DNA typically winds around eight histone proteins (e.g., histone octamer 104). The structural unit of the DNA around the histones is a nucleosome. Histones may carry a modification, which can affect gene transcription. Histone modifications include methylations and acetylations. The histone modifications are part of the epigenome. The epigenomic status is different for different types of cells. The structure of DNA and protein inside a cell is called chromatin. Within the chromatin, the DNA itself is also methylated. The protein structure physically opening and closing the chromatin and other DNA modifications contributing to the chromatin structure are also part of the epigenome. Chromatin remodelers are versatile tools that catalyze broad range of chromatin-changing reactions including sliding of an octamer across the DNA (nucleosome sliding), changing the conformation of nucleosomal DNA, and altering the composition of the octamers (histone variant exchange). Additionally, chromatin remodelers may remove other chromatin proteins from chromatin.

Histone modifications have various functions in the cell. One function is regulating gene expression. Gene expression may be promoted or inhibited. For example, the amount of H3K4me3 is correlated with transcriptional activity. In some cases, a histone modification may increase chromatin compaction and reduce transcription (e.g., H3K36me3).

### II. Measuring Epigenomic Status

A. Histone Modifications Determined Using cfChIP-Seq

Plasma DNA pool is a mixture of DNA molecules released from various tissues, among which certain molecules would be bound to histone proteins accompanied with certain histone modifications. Histone proteins include H1 (linker histones), H2A/B, H3, and H4 (core histones). DNA molecules together with histone proteins would form nucleosome structures (Zhou et al. Nat Struct Mol Biol. 2019; 26:3-13). The coiling of DNA around histones is largely due to electrostatic affinity between the positively charged histones and the negatively charged phosphate backbone of DNA. Histone modifications include but are not limited to histone methylation, acetylation, phosphorylation, and ubiquitylation, etc. (Barth et al. Trends Biochem. Sci. 2010; 35:618-626). Histone methylation could occur at different lysine residues of a histone. The methylation of each lysine residue can involve one, two, or three methyl groups so that the lysine residue would be mono-, di-, or tri-methylated, respectively. Examples of histone methylation include but not limited to the tri-methylation of the lysine (K) residue 4 at the N terminus of histone H3 (H3K4me3), mono-methylation of the lysine (K) residue 4 at the N terminus of histone H3 (H3K4me1) for transcriptional activation, H3K27me3 and H3K9me3 for transcriptional inactivation, and H3K36me3 associated with transcribed regions in gene bodies. H3K9me2 was reported to be a signal for heterochromatin formation in gene-poor chromosomal regions with tandem repeat structures, such as satellite repeats, telomeres, and pericentromeres. Histone acetylation includes, but not limited to, H3K27ac, H3K9ac, and H3K14ac, etc.

Plasma cfDNA molecules bound by histones with certain modifications may be isolated via chromatin immunoprecipitation. Those immunoprecipitated plasma cfDNA molecules can be analyzed using different technologies. In one embodiment, they can be analyzed by DNA sequencing.

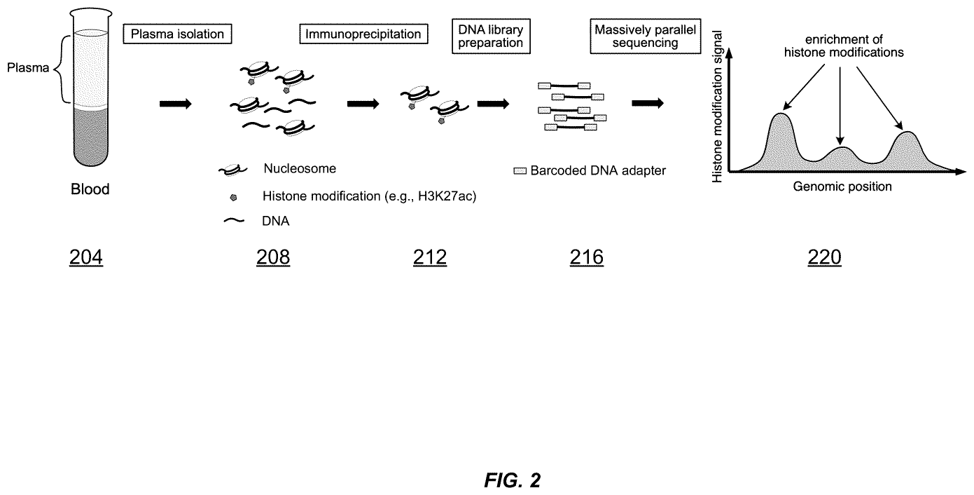

FIG. 2 shows using immunoprecipitation to analyze plasma cfDNA molecules associated with a histone modification. Stage 204 shows the plasma portion of a blood sample. The plasma is isolated. Stage 208 shows components of plasma, including DNA, DNA around histones, and DNA around histones with histone modifications. The plasma cfDNA molecules associated with a histone modification such as H3K27ac are precipitated by magnetic beads conjugated with the H3K27ac antibodies. At stage 212, the precipitated plasma cfDNA molecules are shown. At stage 216, the DNA library is prepared, and the DNA molecules are attached to barcoded adapters. Precipitated cfDNA molecules were analyzed by next generation sequencing (e.g., Illumina NextSeq 500). Sequencing reads can be aligned to a human reference genome GRCh37 (hg19), using for example Bowtie2 (Langmead et al. Nat Methods. 2012; 9:357-359). In some embodiments, one could use, but not limited to, SOAP2 (Li et al. Bioinformatics. 2009; 25:1966-67), Burrows-Wheeler Aligner (BWA) (Li et al. Bioinformatics. 2009; 25:1754-60), BLAT (Kent. Genome Res. 2002:12:656-664), BLAST (Zhang et al. J Comput Biol. 2000; 7:203-14), BFAST (Homer N et al. PLoS One. 2009; 4:e7767), MOSAIK (Lee et al. PLoS One. 2014; 9:e90581), etc. Stage 220 shows a plot of histone modification signal (y-axis) versus genomic position (x-axis). The sequencing depth (or sequencing read density) at a particular genomic region signifies the degree of H3K27ac modification present at that region across different cell types. The higher the sequencing depth at a particular region, the more H3K27ac modifications can potentially be identified. If such H3K27ac modifications were specific to a particular cell type at a particular region, sequencing depth at such a region can be used for determining the amount of cfDNA molecules carrying H3K27ac from that cell type. In one embodiment, the sequencing depth can be normalized and corrected by sequencing biases and/or noise resulting from unspecific bindings. In some examples, the sequencing depth related to chromatin immunoprecipitation assay followed by sequencing (i.e., ChIP-seq) can be used to define histone modification signals or ChIP signals.

B. Selected End Motifs Indicate Histone Modifications

Using fragmentomic features, including but not limited to plasma DNA end motifs and sizes, we developed new approaches for analyzing histone modifications in plasma without the requirement of immunoprecipitation. The regions relatively enriched with histone modifications would generate differential fragment end motif patterns when compared with those regions that lack histone modifications. Thus, the patterns of fragment end motifs could be used for deducing histone modifications. End motif could be defined as one or more nucleotides at one end of a cell-free DNA fragment. The number of nucleotides (nt) at each of fragment ends used for analysis could be, for example, but not limited to, 1 nt, 2 nt, 3 nt, 4 nt, 5 nt, 6 nt, 7 nt, 8 nt, 9 nt, and 10 nt or above. Plasma DNA fragment size could be measured in various ways. In one embodiment, plasma DNA fragment size can be measured by the number of nucleotides present in a plasma DNA molecule. In another embodiment, plasma DNA fragment size can be measured using paired-end sequencing, aligning the sequences to a genome, and then deducing the size from the genome coordinates of the aligned sequences. In embodiments, tissue- or disease-specific histone modification levels are deduced from cfDNA end motif or size frequency, or etc., enabling the monitoring of the physiology or pathology of one or more tissues, or the detection of monitoring of disease status.

The regions with histone modifications may include but not limited to repetitive regions, X chromosome inactivation regions, chromatin structures [e.g., open and closed chromatin structures], pseudogenes, CTCF, DNase I hypertensive sites [DHS], actively transcribed regions and inactively transcribed regions, G quadruplex, etc. For example, selected end motifs in a region with a DNase I hypersensitive site may be used for informing the amount of histone modifications associated with that DNase I hypersensitive site. As another example, sizes of DNA fragments in an X chromosome inactivation region may inform the amount of histone modifications of X-chromosomal genes.

Particular regions may be associated with a particular tissue type. In some instances, a certain property of the region may occur more often for a particular tissue type. As an example, a region being open chromatin (i.e., a large gap between histones) may occur more often for a particular tissue type than other tissue types. Other properties may include the region being a repetitive region, an X chromosome inactivation region, a closed chromatin structure, a pseudogene, CTCF, DHS, actively transcribed region, inactively transcribed region, or G quadruplex. Particular regions may be associated with a specific particular tissue type and no other tissue types. In other embodiments, particular regions may be associated with several different tissue types. The prevalence of that region property may be related to the contribution of the particular tissue type and the relative strength of the particular tissue to be associated with the region property. Deconvolution may be used to determine the tissue contribution from these regions, similar to what is described for histone modifications below.

1. Determining End Motifs Associated with Histone Modifications

Different histone modifications may confer different accessibilities of DNA nucleases, thus resulting in the characteristic fragmentations. Selective cleavage of DNA by nucleases through cfDNA fragmentation occurs in TSS and CpG islands, which have a particular epigenetic status (Han et al., Genome Res. 2021:31:2008-2021). Fragmentation patterns of cell-free DNA may be informative for inferring the histone modifications present in plasma DNA molecules. In embodiments, we analyzed nucleases cutting preference for cfDNA within regions of interest, which could be indicated by the pattern of cfDNA end motifs. The fragment end motif could be defined by one or more nucleotides at one end of a cell-free DNA fragment. For example, we determined the proportions of cfDNA molecules carrying a particular 4-mer end motif (a total of 256 types).

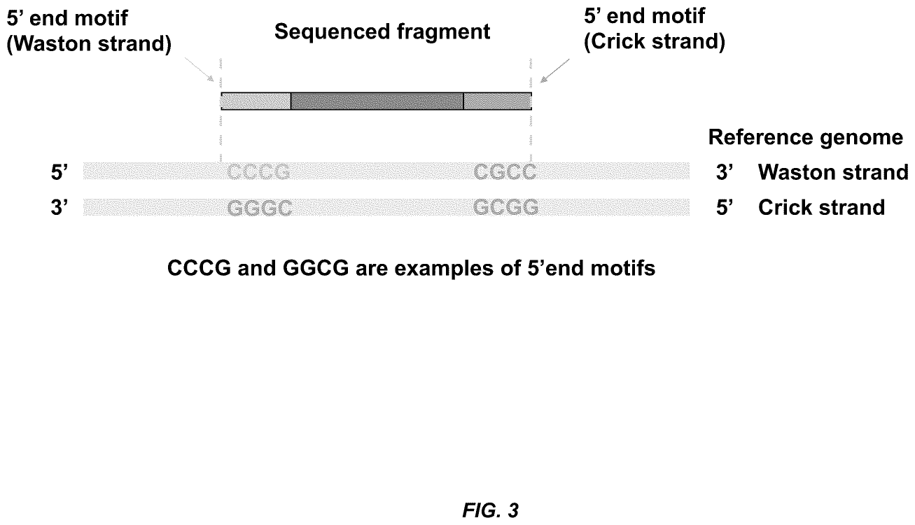

FIG. 3 shows an illustration of the end motif of a fragment. Each nucleotide can be one of four nucleotides: A, C, G, T. For an end motif of four nucleotides, there are 4⁴ (i.e., 256) arrangements. A 4-mer end motif was defined as the four nucleotides at 5′ end of a cfDNA molecule.

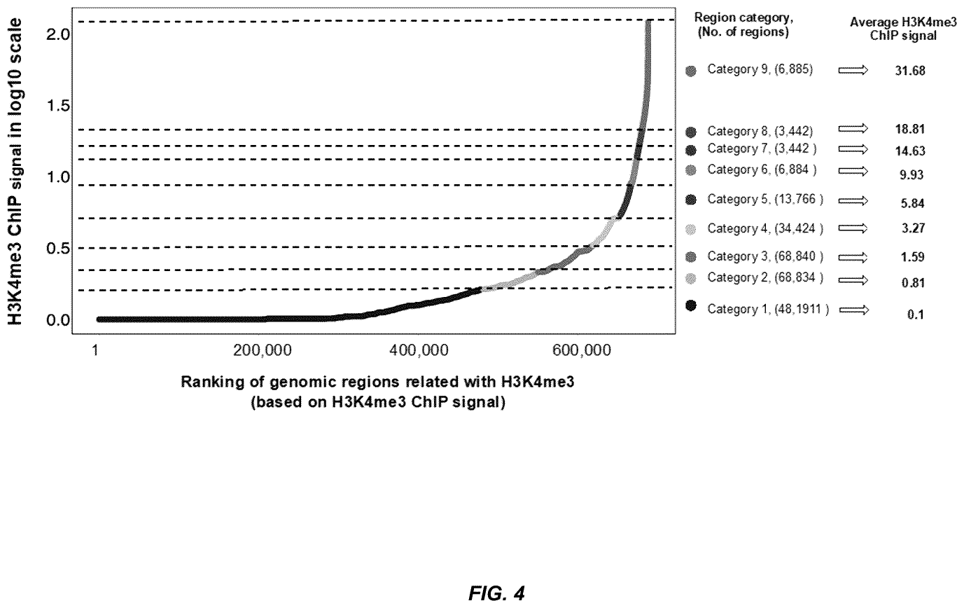

Regions involving histone modifications may be grouped into different categories according to the magnitudes of ChIP signal. FIG. 4 is a graph defining categories of H3K4me3 regions with different levels of H3K4me3 ChIP signal. The y-axis is the H3K4me3 signal with a log₁₀ scale. The x-axis shows the ranking of genomic regions related with H3K4me3. A higher ranking indicates a higher signal. The regions were first sorted according to the magnitudes of ChIP signal and then were empirically classified into 9 categories.

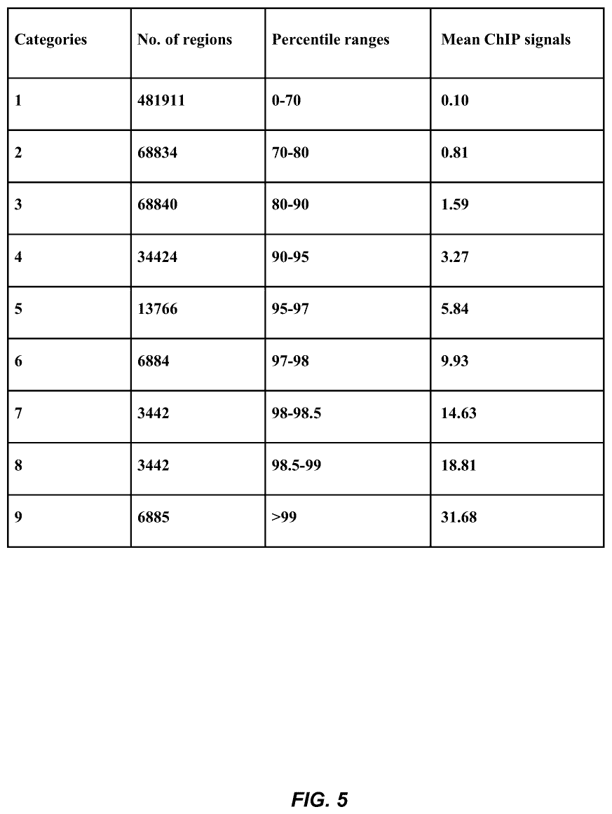

FIG. 5 is a table showing an example definition of categories of H3K4me3 regions using H3K4me3 ChIP-seq analysis of pregnant samples. The first column shows the category identification. The second column shows the number of regions in the category. The third column shows the percentile range for the magnitude of ChIP signals in the regions of the category. The fourth column shows the mean ChIP signal in the regions of the category. As shown in FIG. 5, we empirically classified regions associated with H3K4me3 into 9 categories according to the percentile ranges in terms of strength of ChIP signals. The strength of ChIP signal of a region could be a mean value of FPKM across 12 pregnant samples that were subjected to H3K4me3 ChIP-seq analysis. For instance, a percentile range of ChIP signal of 0 to was defined as category 1, with a mean ChIP signal of 0.10; a percentile range of ChIP signal of 70 to 80 was defined as category 2, with a mean ChIP signal of 0.81; a percentile range of ChIP signal of 80 to 90 was defined as category 3, with a mean ChIP signal of 1.59; a percentile range of ChIP signal of 90 to 95 was defined as category 4, with a mean ChIP signal of 3.27; a percentile range of ChIP signal of 95 to 97 was defined as category 5, with a mean ChIP signal of 5.84; a percentile range of ChIP signal of 97 to 98 was defined as category 6, with a mean ChIP signal of 9.93; a percentile range of ChIP signal of 98 to 98.5 was defined as category 7, with a mean ChIP signal of 14.63; a percentile range of ChIP signal of 98.5 to 99 was defined as category 8, with a mean ChIP signal of 18.81; a percentile range of ChIP signal of 99 or above was defined as category 9, with a mean ChIP signal of 31.68.

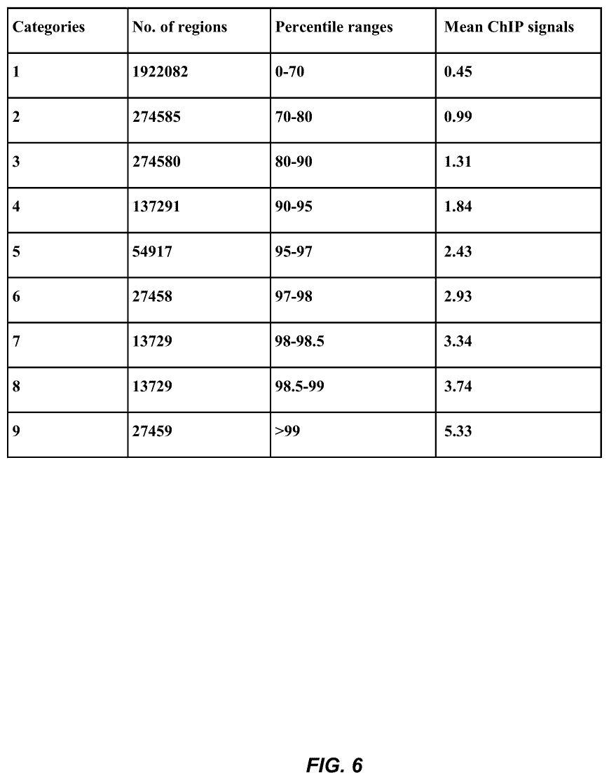

FIG. 6 shows a table showing an example definition of categories of H3K27ac regions using H3K27ac ChIP-seq analysis of pregnant samples. The table in FIG. 6 follows the same format as the table in FIG. 5. As shown in FIG. 6, we empirically classified regions associated with H3K27ac into 9 categories according to the percentile ranges in terms of strength of ChIP signals. The strength of ChIP signal of a region could be a mean value of FPKM across 19 pregnant samples that were subjected to H3K27ac ChIP-seq analysis. For instance, a percentile range of ChIP signal of 0 to 70 was defined as category 1, with a mean ChIP signal of 0.45; a percentile range of ChIP signal of 70 to 80 was defined as category 2, with a mean ChIP signal of 0.99; a percentile range of ChIP signal of 80 to 90 was defined as category 3, with a mean ChIP signal of 1.31; a percentile range of ChIP signal of 90 to 95 was defined as category 4, with a mean ChIP signal of 1.84; a percentile range of ChIP signal of 95 to 97 was defined as category with a mean ChIP signal of 2.43; a percentile range of ChIP signal of 97 to 98 was defined as category 6, with a mean ChIP signal of 2.93; a percentile range of ChIP signal of 98 to 98.5 was defined as category 7, with a mean ChIP signal of 3.34; a percentile range of ChIP signal of 98.5 to 99 was defined as category 8, with a mean ChIP signal of 3.74; a percentile range of ChIP signal of 99 or above was defined as category 9, with a mean ChIP signal of 5.33. We could also use other methods to define region categories, including but not limited to k-means clustering analysis.

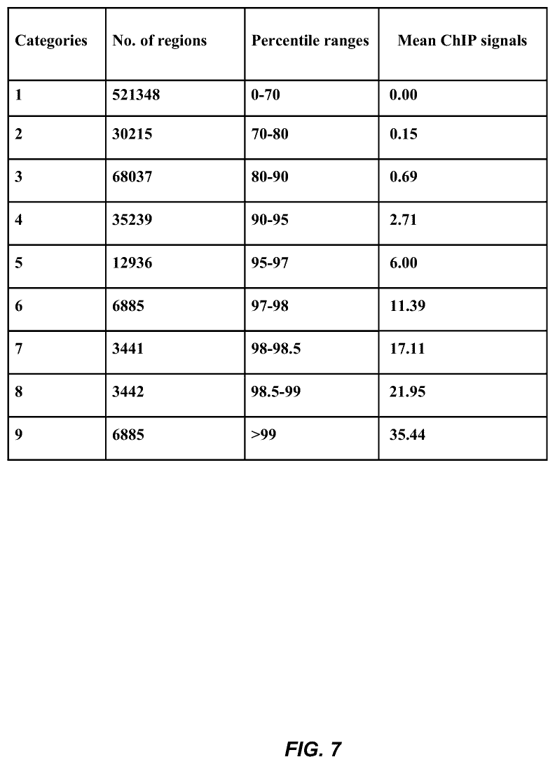

FIG. 7 is a table showing an example definition of categories of H3K4me3 regions using H3K4me3 ChIP-seq analysis of samples from non-pregnant, healthy subjects. The table in FIG. 7 follows the same format as the table in FIG. 5. FIG. 7 shows building a reference using non-pregnant healthy samples that were subjected to ChIP-seq analysis. As shown in FIG. 7, we empirically classified regions associated with H3K4me3 into 9 categories according to the percentile ranges in terms of strength of ChIP signals. The strength of ChIP signal of a region could be a mean value of FPKM across 4 healthy samples that were subjected to H3K4me3 ChIP-seq analysis. For instance, a percentile range of ChIP signal of 0 to 70 was defined as category 1, with a mean ChIP signal of 0.00; a percentile range of ChIP signal of 70 to 80 was defined as category 2, with a mean ChIP signal of 0.15; a percentile range of ChIP signal of 80 to was defined as category 3, with a mean ChIP signal of 0.69; a percentile range of ChIP signal of 90 to 95 was defined as category 4, with a mean ChIP signal of 2.71; a percentile range of ChIP signal of 95 to 97 was defined as category 5, with a mean ChIP signal of 6.00; a percentile range of ChIP signal of 97 to 98 was defined as category 6, with a mean ChIP signal of 11.39; a percentile range of ChIP signal of 98 to 98.5 was defined as category 7, with a mean ChIP signal of 17.11; a percentile range of ChIP signal of 98.5 to 99 was defined as category 8, with a mean ChIP signal of 21.95; a percentile range of ChIP signal of 99 or above was defined as category 9, with a mean ChIP signal of 35.44.

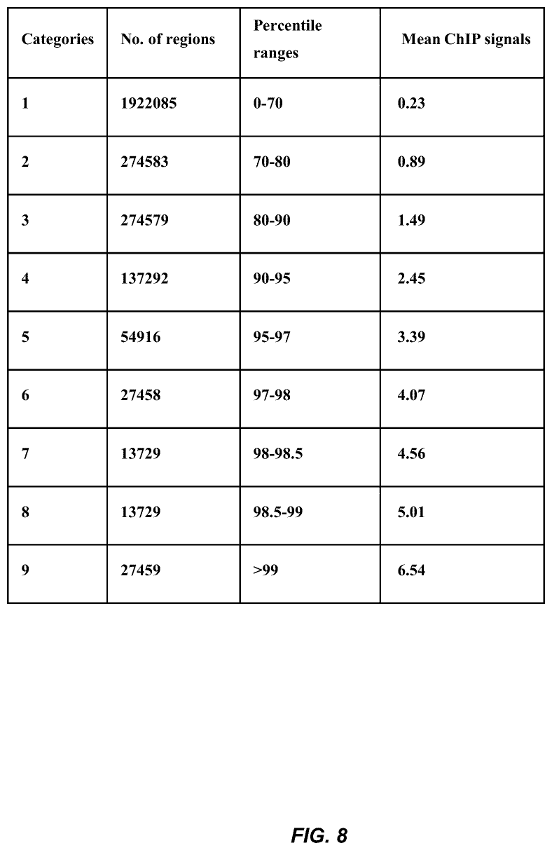

FIG. 8 is a table showing an example definition of categories of H3K27ac regions using H3K27ac ChIP-seq analysis of samples from non-pregnant, healthy subjects. The table in FIG. 8 follows the same format as the table in FIG. 5. As shown in FIG. 8, we empirically classified regions associated with H3K27ac into 9 categories according to the percentile ranges in terms of strength of ChIP signals. The strength of ChIP signal of a region could be a mean value of FPKM across 6 healthy samples that were subjected to H3K27ac ChIP-seq analysis. For instance, a percentile range of ChIP signal of 0 to 70 was defined as category 1, with a mean ChIP signal of 0.23; a percentile range of ChIP signal of 70 to 80 was defined as category 2, with a mean ChIP signal of 0.89; a percentile range of ChIP signal of 80 to 90 was defined as category 3, with a mean ChIP signal of 1.49; a percentile range of ChIP signal of 90 to 95 was defined as category 4, with a mean ChIP signal of 2.45; a percentile range of ChIP signal of 95 to 97 was defined as category 5, with a mean ChIP signal of 3.39; a percentile range of ChIP signal of 97 to 98 was defined as category 6, with a mean ChIP signal of 4.07; a percentile range of ChIP signal of 98 to 98.5 was defined as category 7, with a mean ChIP signal of 4.56; a percentile range of ChIP signal of 98.5 to 99 was defined as category 8, with a mean ChIP signal of 5.01; a percentile range of ChIP signal of 99 or above was defined as category 9, with a mean ChIP signal of 6.54.

We analyzed 4-mer end motif frequencies across the 9 categories defined according to the different levels of H3K4me3 signal for samples without immunoprecipitation.

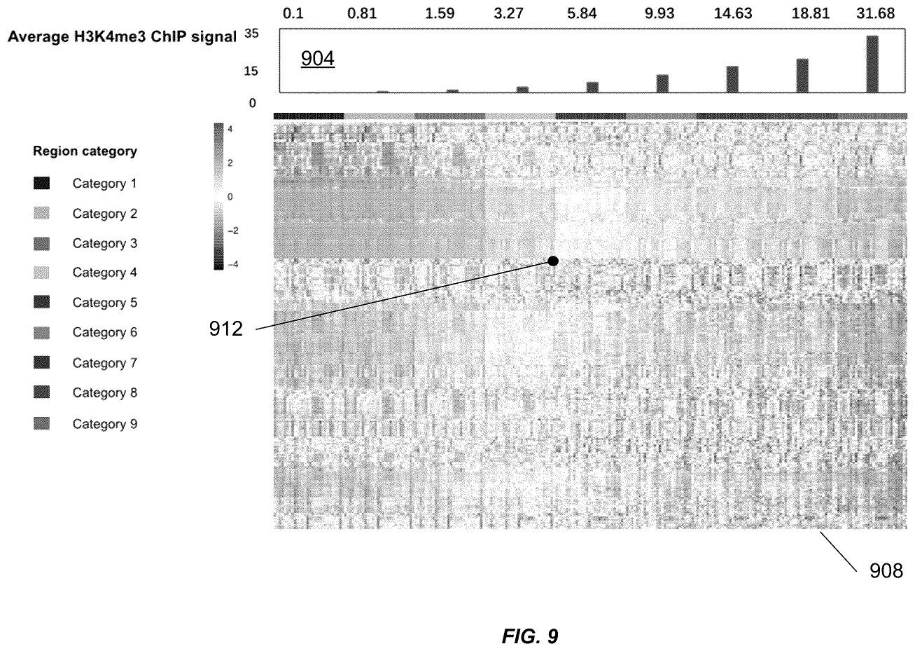

FIG. 9 shows a heatmap of motif frequencies in regions with different levels of H3K4me3 ChIP signals for plasma DNA sequencing results. Graph 904 shows the average H3K4me3 ChIP signal. The y-axis shows the average H3K4me3 ChIP signals. The x-axis shows the 9 categories of H3K4me3 regions. The x-axis categories align with the regions in heatmap 908. The y-axis of heatmap 908 corresponds to different 4-mer end motifs. The more red a point is, the higher the end motif frequency is in one region of one sample compared with that in the other combinations of region categories and samples. The more blue a point is, the lower the end motif frequency is in one region of one sample compared with that in the other combinations of region categories and samples. As shown in FIG. 9, the end motifs frequencies from plasma DNA sequencing data without immunoprecipitation varied according to the strengths of ChIP signal obtained from plasma DNA sequencing data with immunoprecipitation, suggesting the possibility for deducing plasma DNA histone modifications on the basis of end motifs of plasma DNA molecules without immunoprecipitation. Point 912 is a point where four unequal sized quadrants intersect. The upper right quadrant is more red. The upper left quadrant is more blue. The lower right quadrant is more blue. The lower left quadrant is more red.

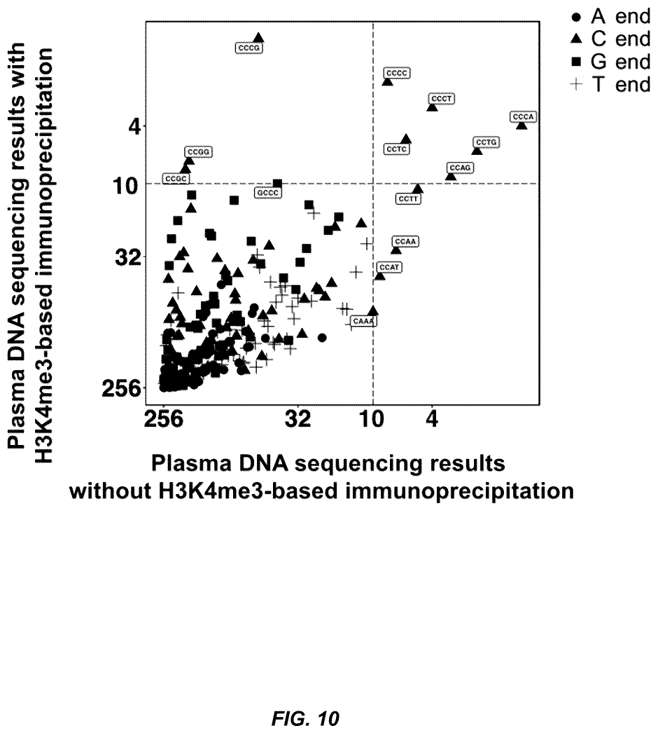

FIG. 10 is a graph of a comparison of end motif frequencies ranking between plasma DNA sequencing results with and without H3K4me3-based immunoprecipitation. The y-axis shows the ranking of end motifs from cfChIP-seq for H3K4me3 from 256 to 1, with 1 representing the most frequent end motif. The x-axis shows the ranking from 256 to 1 of end motifs resulting from conventional cfDNA sequencing on a plasma sample, without adding an antibody specific for H3K4me3 modification. The shape of the data point indicates the end nucleotide (circle for A, triangle for C, square for G, plus for T).

FIG. 10 shows that a number of 4-mer end motifs appeared to be overrepresented in H3K4me3-mediated immunoprecipitated plasma DNA sequencing results, including but not limited to GCGG, GCGC, CGCG, CCGC, CCGA, TCCG, CCGT, GGCG, CCGG, TGCG, GCCG, CTCG, GCGA, TCGG, CGGC, TCGC, CGGG, CGCC, ACCG, AGCG, CGGA, GGGC, GCGT, CACG, etc. (i.e., motifs where the ranking on the y-axis is a lower number than the ranking on the x-axis), compared with plasma DNA without immunoprecipitation. Overrepresented end motifs were considered to those end motifs above the diagonal line y=x and with x−y≥100. Those overrepresented end motifs may be suggestive of the presence of histone modifications (H3K4me3). In another embodiment, underrepresented motifs (i.e., motifs where the ranking on the y-axis is a higher number than the ranking on the x-axis) can be used.

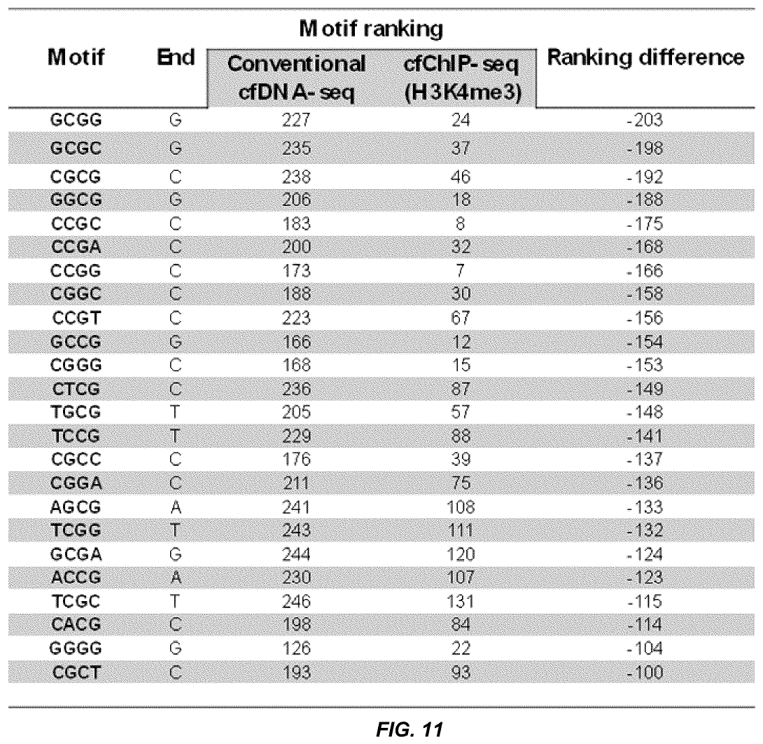

FIG. 11 shows a table of 24 end motifs with the greatest ranking differences between conventional cfDNA sequencing and cfChIP-seq for H3K4me3 histone modification. The first column shows the motif. The second column shows the nucleotide at the very end of the fragment (i.e., the first nucleotide listed in the first column). The third column shows the ranking of the motif in conventional cfDNA sequencing, with 1 being the most frequent and highest ranking and 256 being the least frequent and lowest ranking. The fourth column shows the ranking of the motif in cfChIP-seq for the H3K4me3 histone modification. The fifth column shows a ranking difference when taking the cfChIP-seq ranking and subtracting the conventional cfDNA sequencing ranking. The columns are ordered by the magnitude of the ranking difference. The data was acquired from multiple health subjects.

The results also show that many of the end motifs with higher rankings in cfChIP-seq have C and G nucleotides adjacent to each other. H3K4me3 sites appear to be enriched with CG sequences.

Accordingly, the end motifs with the largest ranking difference occur at a higher rate in the regions associated with H3K4me3 than occur without cfChIP, genome-wide, or relative to a random group of DNA fragments.

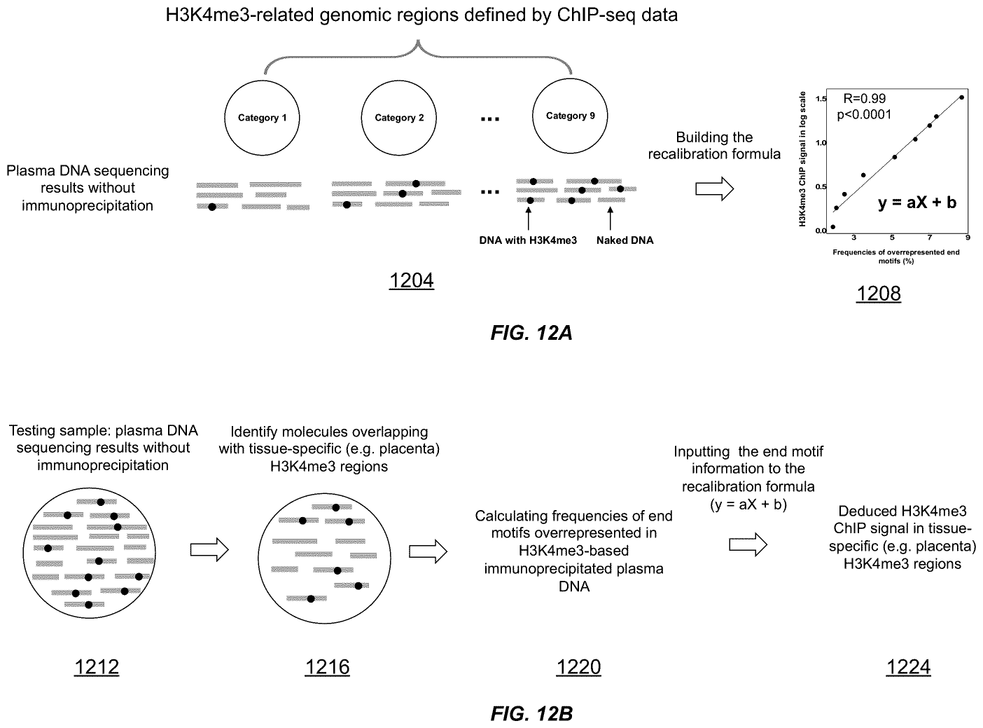

FIG. 12A and FIG. 12B illustrate the use of end motif patterns to deduce plasma DNA histone modifications signal for plasma DNA sequencing results without immunoprecipitation. FIG. 12A shows building the recalibration formula with the frequency of overrepresented end motifs and the level of H3K4me3 ChIP signals in the 9 categories. In stage 1204, the regions involving H3K4me3 were grouped into different categories according to the magnitudes of ChIP signal. In one embodiment, one could divide regions into 9 categories based on the magnitudes of ChIP signal of each region. After we obtained region categories with different ChIP signals, the end motif patterns (e.g., aggregated frequency of cfDNA molecules with overrepresented end motifs from plasma DNA sequencing results without immunoprecipitation) in each region category may be used to correlate with the H3K4me3 ChIP signals. In stage 1208, based on the correlation between fragment end motifs and ChIP signals, a recalibration formula can be determined. A linear formula is shown as an example of a recalibration formula, but non-linear formulas may also be used.

FIG. 12B shows how the recalibration formula can be used to infer the ChIP signals in other regions (e.g., placenta-specific H3K4me3 regions) according to the corresponding end motif information of those regions (i.e., deduced ChIP signal). At stage 1212, plasma DNA are sequenced without immunoprecipitation. At stage 1216, molecules overlapping with tissue-specific (e.g., placenta) H3K4me3 regions are identified. At stage 1220, frequencies of end motifs overrepresented in H3K4me3-based immunoprecipitated plasma DNA are calculated. The end motif information is inputted into the recalibration formula, and at stage 1224, the H3K4me3 ChIP signal is deduced in tissue-specific (e.g., placenta) H3K4me3 regions).

2. Testing Correlation with cfChIP-Seq Signal

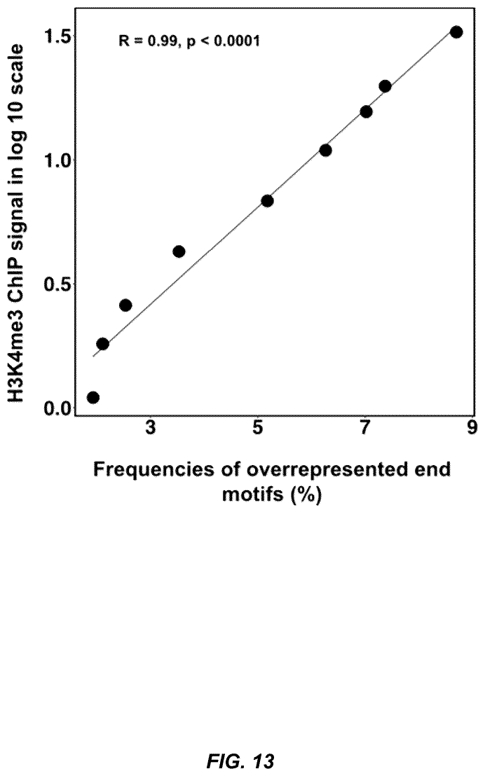

FIG. 13 shows a graph of the correlation between the aggregated abundance of end motifs overrepresented in H3K4me3-based immunoprecipitated plasma DNA and H3K4me3 ChIP signal. The x-axis shows the frequencies of overrepresented end motifs as a percent. The y-axis is the H3K4me3 ChIP signal in log₁₀ scale. FIG. 13 shows that the aggregated abundance of overrepresented end motifs was highly correlated with the H3K4me3 ChIP signals (Pearson's r: P value: <0.0001). The data shows that the use of plasma DNA end motifs can be used for deducing the strength of signals related to certain histone modifications. Hence, one can generate a recalibration formula using a linear regression model, facilitating the deduction of H3K4me3 ChIP signals based on end motifs of plasma DNA molecules without the need of an immunoprecipitation assay. Additionally, the motif frequency can be used to predict H3K4me3 histone modification and any other properties that H3K4me3 histone modification can be used, such as a percentage of DNA from a particular tissue type or a condition of the subject.

A higher frequency of the 24 end motifs from FIG. 11 would be expected to correlate with a higher cfChIP-seq signal. To test this hypothesis, we divided the cfChIP-seq signal into a different number of groups based on the height of the peaks.

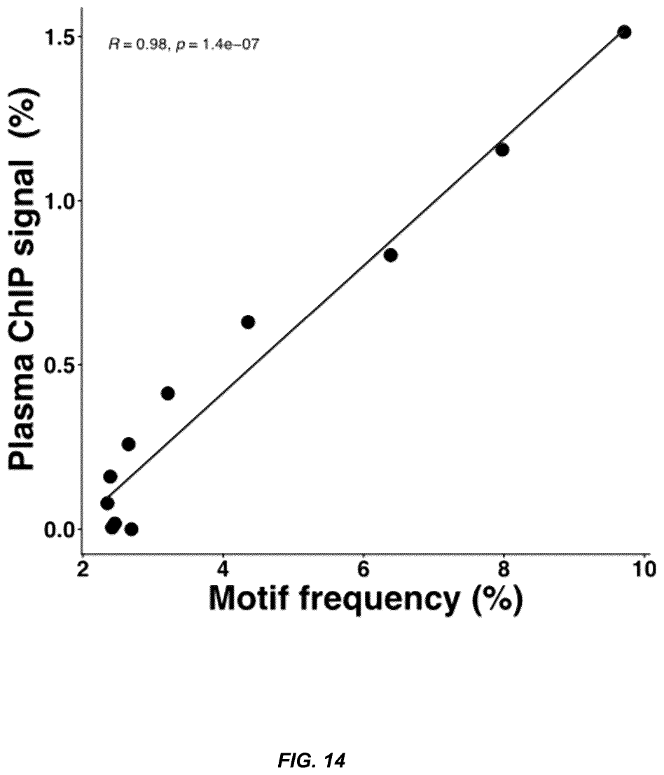

FIG. 14 is a graph showing the correlation between the cfChIP signal and the end motif frequency for 11 peak groups. Each dot (data point) corresponds to a different peak group of the 11 peak groups. Because the peaks correspond to the signal value, the signal increases with successive peak groups. The x-axis shows the aggregate frequency of the end motifs in the peak group relative to all motifs for the specific genomic region being analyzed. The end motif frequency for a peak group is for the specific genomic region associated with the peak group. The y-axis shows the average signal from cfChIP-seq for H3K4me3 histone modification for each peak group. As an example, each peak group can include a number of peaks as shown in FIGS. 5, 6, 7, and 8.

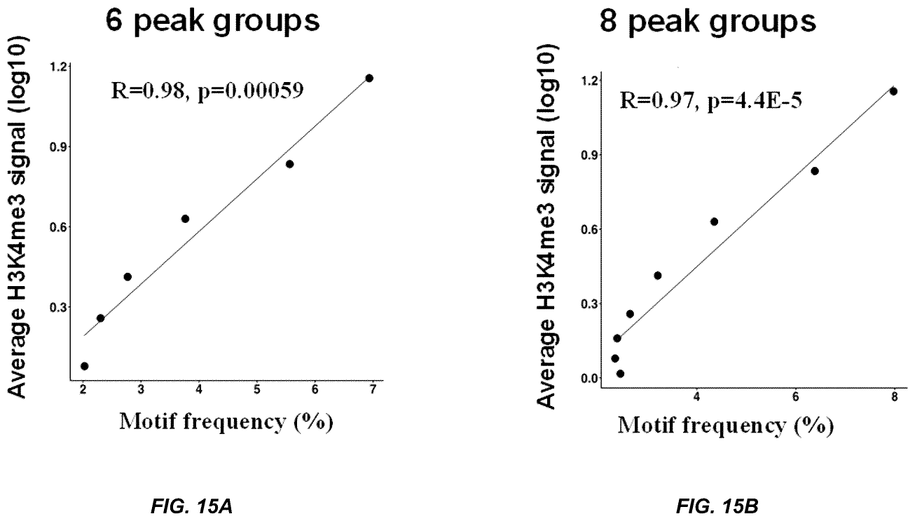

FIG. 15A is a graph showing the correlation between the cfChIP signal and the end motif frequency for 6 peak groups. The y-axis shows the average signal from cfChIP-seq for H3K4me3 histone modification for each peak group. The x-axis shows the frequency of the end motifs, similar to FIG. 14. Each dot represents one of the 6 peak groups. The end motif frequency for a peak group is for the specific genomic region associated with the peak group. The end motifs for FIG. 15A included all 24 end motifs identified in FIG. 11. This graph shows a high correlation with an R value of 0.98 and a p value of 0.00059. This graph suggests that using the frequency of the top 24 end motifs is correlated with the cfChIP-seq signal of the H3K4me3 histone modification. The graph also shows that grouping end motifs into six peak groups can maintain the correlation with the cfChIP-seq signal.

FIG. 15B is a graph showing the correlation between the cfChIP signal and the end motif frequency for 8 peak groups. The y-axis shows the average signal from cfChIP-seq for H3K4me3 histone modification for each peak group. The x-axis shows the frequency of the end motifs, similar to FIG. 14. Each dot represents one of the 8 peak groups. The end motif frequency for a peak group is for the specific genomic region associated with the peak group. The end motifs for FIG. 15A included all 24 end motifs identified in FIG. 11. This graph shows a high correlation with an R value of 0.97 and a p value of 4.4e-05. This graph suggests that using the frequency of the top 24 end motifs is correlated with the cfChIP-seq signal of the H3K4me3 histone modification. The graph also shows that grouping end motifs into eight peak groups can maintain the correlation with the cfChIP-seq signal.

FIGS. 14, 15A, and 15B show that end motif frequency correlates with the signal from cfChIP-seq signal peaks within a group. The correlation is high even when varying the number of peak groups.

### III. Using Sequence Motifs to Analyze Epigenomic Status

Because end motif frequency can identify epigenome status and different cells have different epigenome statuses, end motif frequency may be used to identify the tissue origin, determine a fractional concentration of a tissue in the sample, estimate characteristics of tissues, or determine levels of a disorder. End motif frequencies can also measure amounts of histone modifications.

A. Estimating Fractional Concentration of Tissue of Origin

The genomic regions where H3K4me3 signals are high for placenta are known (FIG. 4). Additionally, end motif frequencies for these genomic regions are known for different peak groups (FIG. 14). An overall end motif frequency is determined for the 24 end motifs in the various genomic regions corresponding to the 11 peak groups. Based on the end motif frequency, an H3K4me3 signal is predicted. The equation describing the linear relationship in FIG. 14 is log(average H3K4me3 signal)=a*(end motif frequency)+b.

1. Results

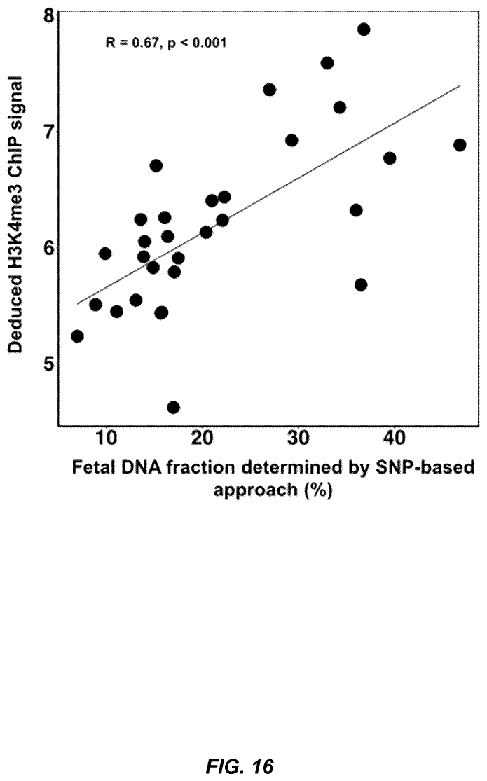

FIG. 16 is a graph of the correlation between the H3K4me3 ChIP signal in placenta-specific H3K4me3 regions deduced by end motifs and fetal DNA fraction determined by SNP-based approach. The x-axis is the fetal DNA fraction as a percent determined by the SNP-based approach. The y-axis is the deduced H3K4me3 ChIP signal using end motifs. The deduced H3K4me3 ChIP signals by using end motifs was correlated with the fetal DNA fraction in plasma DNA of pregnant women (Pearson's r: 0.67; P value: <0.001).

2. Example Method for Determining Fractional Concentration

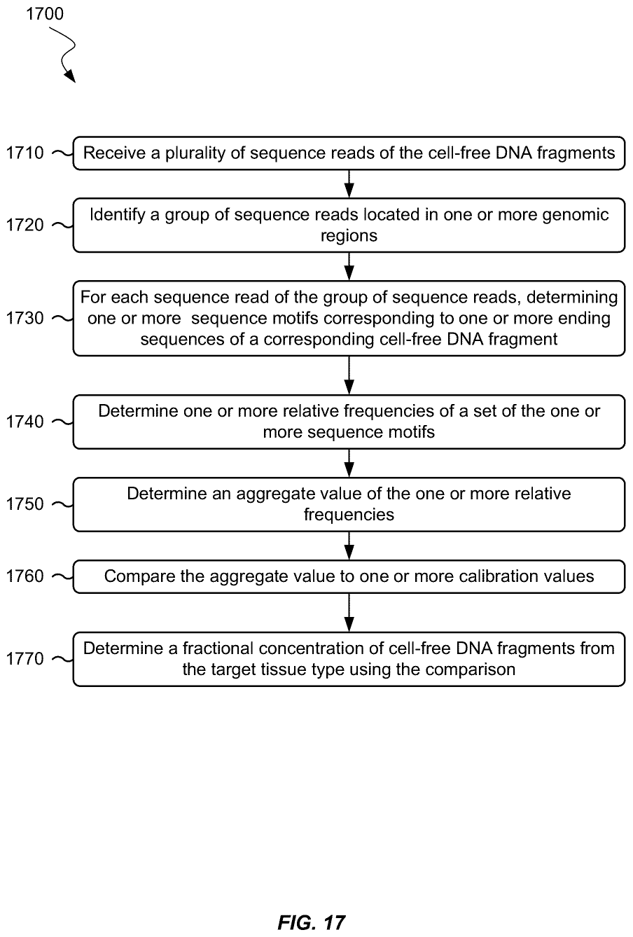

FIG. 17 is a flowchart of an example process 1700 associated with determining a fractional concentration of cell-free DNA fragments in a biological sample. The biological sample may include cell-free DNA fragments. The biological sample may be any biological sample described herein, including plasma or serum. In some implementations, one or more process blocks of FIG. 17 may be performed by a system (e.g., measurement system 5900). In some implementations, one or more process blocks of FIG. 17 may be performed by another device or a group of devices separate from or including the system. Additionally, or alternatively, one or more process blocks of FIG. 17 may be performed by one or more components of measurement system 5900, such as assay 5908, assay device 5910, detector 5920, logic system 5930, local memory 5935, external memory 5940, storage device 5945, and/or processor 5950.

At block 1710, a plurality of sequence reads of the cell-free DNA fragments is received. The plurality of sequence reads include ending sequences corresponding to ends of the plurality of cell-free DNA fragments.

In some embodiments, process 1700 may include sequencing the cell-free DNA fragments in the biological sample to obtain the plurality of sequence reads. In embodiments, the volume of the biological sample may be 100 μl or less, including 80 to 100 μl to 80 μl or 30 to 50 μl. The biological sample may use a volume smaller than the volume used in cfChIP-seq.

In some embodiments, process 1700 may include probe-based techniques to measure the amount of motifs. Techniques may include qPCR, digital PCR, digital droplet PCR, etc. As an example, cfDNA molecules can be subjected to the process of DNA end pair, A-tailing, and common adaptor ligation. The adaptor-ligated molecules can be partitioned, e.g., into different reactions, such as droplets. A pair of PCR primers can be designed in a way that one primer could bind to the common adaptor region and the other could bind to the specific region of interest. DNA molecules would be amplified inside a reaction (e.g., droplet) by the pair of PCR primers. The fluorescent probe specific to a certain end motif can be hydrolyzed and emit fluorescent signals, thus enabling the detection of the presence of a specific motif as well as the quantification of a specific motif. For digital PCR, the number of reactions positive for a particular end motif can be counted and used to determine the amount of DNA fragments with that end motif in the region analyzed. For real-time PCR, the intensity of each signal can be used as a measure of an amount of DNA fragments ending with a particular motif. The two intensities can be compared to each other.

At block 1720, a group of sequence reads located in one or more genomic regions is identified. Each of the one or more genomic regions has a histone modification associated with a target tissue type. The target tissue type may include the placenta, liver, heart, neutrophils, monocytes, B cells, adipose, NK cells, or any tissue type described herein. The histone modification may be H3K4me3, H3K4me1, H3K4me2, H3K27me3, H3K27ac, H3K36me3, H3K9me2, H3K9me3, H3S10P, H3R2me, H3T2P, H3K14ac, H3K9ac, H3K79me2, H3K79me3, H4K5ac, H4K8ac, H4K12ac, H4K16ac, H4K20me, H2BK120ub, H2AK119ub. The one or more genomic regions may include transcription start sites, promoter regions, enhancer regions, super enhancer regions, gene bodies, repetitive sequences, satellite repeats, telomeres, pericentromeres, mitotic chromosomes, transcriptional end sites. exon, intron, insulator, etc. The one or more genomic regions may have amounts of histone modification that are statistically significantly different from the amounts of histone modifications in other genomic regions or the average amount of modifications in other genomic regions or across all genomic regions. The sequence reads may be aligned to a reference genome (e.g., human reference genome) to determine if the sequence reads are located in the one or more genomic regions.

At block 1730, one or more sequence motifs corresponding to one or more ending sequences of a corresponding cell-free DNA fragment are determined for each sequence read of the group of sequence reads. The one or more sequence motifs may be correspond to a single nucleotide, a two-nucleotide sequence, a three-nucleotide sequence, a four-nucleotide sequence, a five-nucleotide sequence, a six-nucleotide sequence, a seven-nucleotide sequence, an eight-nucleotide sequence, or a sequence having more than eight nucleotides. The one or more sequence motifs may each have the same number of nucleotides. In some embodiments, the sequence motif includes the nucleotide at the end of the cell-free DNA fragment. The sequence motif may be at the 5′ end of the cell-free DNA fragment. In some embodiments, the sequence motif may be at the 3′ end. In embodiments, the one or more sequence motifs may include sequence motifs at the 3′ end and at the 5′ end. If a whole fragment is sequenced, two sequence motifs may be determined.

At block 1740, one or more relative frequencies of a set of the one or more sequence motifs are determined. The set of the one or more sequence motifs occurs at a higher rate in chromatin immunoprecipitation followed by sequencing (ChIP-seq) for the histone modification associated with the one or more genomic regions than in sequencing without chromatin immunoprecipitation. The chromatin immunoprecipitation may be cell-free chromatin immunoprecipitation followed by sequencing (cfChIP-seq) or may be cellular chromatin immunoprecipitation followed by sequencing. Sequencing without chromatin immunoprecipitation may include genome-wide sequencing. The set of the one or more sequence motifs correspond to sequence motifs having a similar relative frequency, such as a peak group in FIG. 14, 15A, or 15B. The one or more sequence motifs may, for example, be any of the sequence motifs in FIG. 11. The relative frequency may be a motif frequency in FIG. 14, 15A, or 15B. The set of the one or more sequence motifs may include 1 to 5, 5 to 10, 11 to 15, 15 to 20, or 20 to 25 sequence motifs. A relative frequency for each sequence motif may be determined. In other embodiments, one relative frequency may be determined for multiple sequence motifs, including the set of the one or more sequence motifs. Determining the set of sequence motifs is described below.

At block 1750, an aggregate value of the one or more relative frequencies is determined. Example aggregate values are described throughout the disclosure, e.g., including an entropy value (a motif diversity score or variance), a sum of relative frequencies, and a multidimensional data point corresponding to a vector of counts for a set of motifs (e.g., a vector 256 counts for 256 motifs of possible 4-mers or 64 counts for 64 motifs of possible 3-mers). When the set of one or more sequence motifs includes a plurality of sequence motifs, the aggregate value can include a sum of the relative frequencies of the set. In some embodiments, the aggregate value may be an estimation of the histone modifications. The levels of histone modifications can be determined by various types of data, e.g., amounts of end motifs or fragment sizes.

At block 1760, the aggregate value is compared to one or more calibration values. The one or more calibration values are determined from one or more calibration samples whose fractional concentrations of cell-free DNA fragments from the target tissue type are known.

The one or more calibration values may be determined through determining aggregate values for sequence motifs of the one or more calibration samples. For example, the aggregate value determined from the biological sample may be a first aggregate value determined from one or more first relative frequencies. One or more second relative frequencies of the set of the one or more sequence motifs in the one or more genomic regions may be determined for each calibration sample of the one or more calibration samples. A second aggregate value may be determined for the one or more second relative frequencies for each calibration sample of the one or more calibration samples. Each of the one or more second aggregate values may thereby be associated with a known concentration of the calibration samples. The calibration value may include the one or more second aggregate values. For example, the calibration values may be points along a line or a curve relating known concentrations with the second aggregate values.

In some embodiments, the one or more calibration values may be determined from a function relating known concentrations with second aggregate values. The first aggregate value may be inputted into the function to return a fractional concentration. The first aggregate value is then used as the calibration value. The comparison of the aggregate value is comparing the aggregate value to the calibration value used in the function and determining that the aggregate value is the same as the calibration value.

At block 1770, a fractional concentration of cell-free DNA fragments from the target tissue type is determined using the comparison. The fractional concentration may be the known fractional concentration associated with the calibration value, which may have a value close to or equal to the first aggregate value. In some embodiments, the fractional concentration may be determined from a function or a line with the one or more calibration values. The function or line may relate known fractional concentrations to the one or more calibration values. The fractional concentration of the target tissue type can be used to determine characteristics of the tissue type and/or the subject from which the biological sample is obtained.

A classification of a disorder or disease may be determined using the fractional concentration. For example, if the target tissue type is the placenta, the method may further include determining a classification of a pregnancy-associated disorder or a gestational age using the fractional concentration. The fractional concentration may be compared to a cutoff value determined from samples from reference subjects having a certain classification of the pregnancy-associated disorder or having a certain gestational age. A pregnancy-associated disorder may include pre-eclampsia, intrauterine growth restriction, invasive placentation and pre-term birth, hemolytic disease of the newborn, placental insufficiency, hydrops fetalis, fetal malformation, HELLP syndrome, systemic lupus erythematosus, and other immunological diseases of the mother. The pregnancy-associated disorder may be associated with the fetus or the mother.

In some embodiments, a classification of a level of cancer may be determined using the fractional concentration. The fractional concentration may be compared to a cutoff value determined from samples from reference subjects having a certain classification of the level of cancer.

a) Fractional Concentration of a Second Target Tissue Type

In some embodiments, fractional concentrations of multiple tissue types can be determined. Different tissues can show different histone modification amounts in different genomic regions (e.g., as described in section V.A). A biological sample, such as a plasma sample, may have DNA fragments from different tissues. The DNA fragments may therefore include fragments associated with the histone modification in different genomic regions. Each genomic region may have sequence motifs associated with the histone modification. The sequence motifs in different genomic regions can be used to determine fractional concentrations of the different tissues in the biological sample. The amounts of the sequence motifs are correlated with the fractional concentrations of the tissues. The method can be repeated for a second target tissue to determine the fractional concentration of the second target tissue.

For example, the steps described above may be for a first target tissue type. The one or more genomic regions associated with the first target tissue type may be one or more first genomic regions. The group of sequence reads located in the one or more first genomic regions may be a first group of sequence reads. The histone modification in the one or more first genomic regions may be a first histone modification. The set of the one or more sequence motifs may be a set of one or more first sequence motifs. The relative frequency may be a first relative frequency. The aggregate value may be a first aggregate value. The one or more calibration samples may be one or more first calibration samples. The fractional concentration may be a first fractional concentration.

The method may further include identifying a second group of sequence reads located in one or more second genomic regions in a similar manner as block 1720. Each of the one or more second genomic regions may have a second histone modification associated with a second target tissue type. The one or more second genomic regions may be the same as or different from the one or more first genomic regions.

For each sequence read of the second group of sequence reads, one or more second sequence motifs corresponding to the one or more ending sequences of a corresponding cell-free DNA fragment may be determined, similar to block 1730.

One or more second relative frequencies of a set of the one or more second sequence motifs may be determined, similar to block 1740. The set of the one or more second sequence motifs may occur at a higher rate in chromatin immunoprecipitation sequencing for the second histone modification associated with the one or more second genomic regions than in sequencing without chromatin immunoprecipitation. Sequence motifs that appear more frequently in ChIP-sequencing may be used because those sequence motifs may be associated with the second histone modification (similar to FIG. 10). Determining the set of sequence motifs is described below.

A second aggregate value of the one or more second relative frequencies may be determined, similar to block 1750.

The one or more second aggregate values may be compared to one or more second calibration values in a similar manner as block 1760.

The one or more second calibration values may be determined from one or more second calibration samples whose fractional concentrations of DNA fragments from the second target tissue type are known. A second fractional concentration of cell-free DNA fragments from the second target tissue type may be determined using the comparison, similar to block 1770.

b) Determining Sequence Motifs

The set of the one or more sequence motifs can be determined in a manner similar to the procedure described with FIGS. 3, 10, and 11. A first rate of each of the one or more sequence motifs relative to other sequence motifs in cfChIP-sequencing may be determined. The first rate may be a ranking, as with FIG. 10, or a frequency. The frequency may be determined by a ratio of the raw count of the sequence motifs in the set to the count outside the set. A second rate of each of the set of the one or more sequence motifs relative to other sequence motifs in sequencing without chromatin immunoprecipitation. The second rate may be of the same type as the first rate (e.g., ranking, frequency). Each of the set of the one or more sequence motifs may be identified as having a first rate higher than the second rate. The identification may be through using a graphical representation (e.g., FIG. 10) or through determining a difference between rankings or frequencies (e.g., FIG. 11). Each set of the one or more sequence motifs may have a difference above a threshold difference. Sequence motifs not in the set may have a difference below the threshold difference.

Process 1700 may include additional implementations, such as any single implementation or any combination of implementations described herein and/or in connection with one or more other processes described elsewhere herein.

Although FIG. 17 shows example blocks of process 1700, in some implementations, process 1700 may include additional blocks, fewer blocks, different blocks, or differently arranged blocks than those depicted in FIG. 17. Additionally, or alternatively, two or more of the blocks of process 1700 may be performed in parallel.

B. Estimating Characteristic Value of Target Tissue

The values of various characteristics of target tissues can be estimated using sequence motifs associated with histone modifications. The characteristics can describe the health of the tissue, the age of the tissue, or a level of disease in the tissue. For example, the determined characteristic can include a particular gestational age or range (e.g., 8 weeks, 9-12 weeks). In another example, the determined characteristic can be a size or nutrition status of an organ corresponding a particular tissue type.

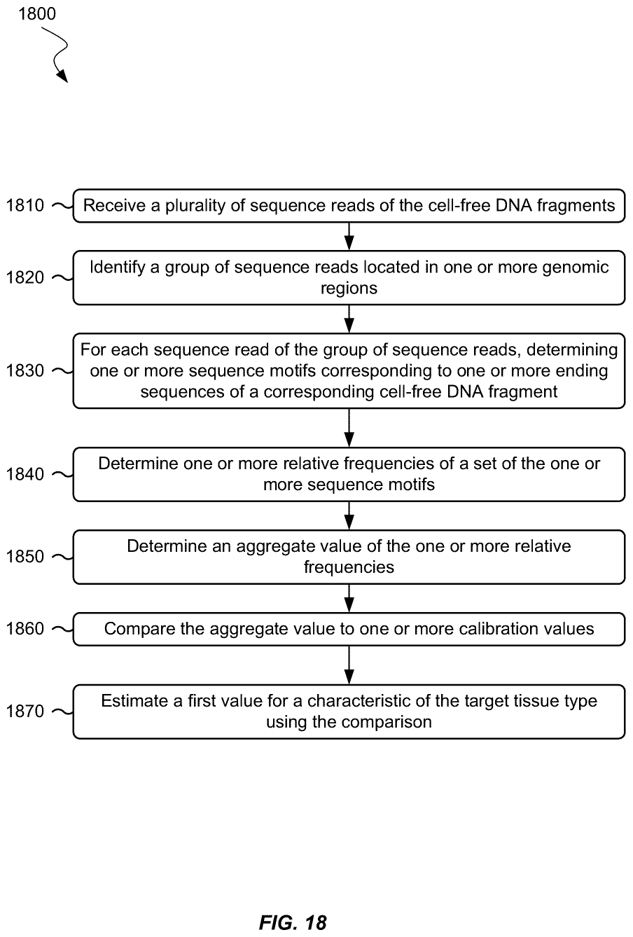

FIG. 18 is a flowchart of an example process 1800 associated with estimating a first value of a characteristic of the target tissue. In some implementations, one or more process blocks of FIG. 18 may be performed by a system (e.g., measurement system 5900). In some implementations, one or more process blocks of FIG. 18 may be performed by another device or a group of devices separate from or including the system. Additionally, or alternatively, one or more process blocks of FIG. 18 may be performed by one or more components of measurement system 5900, such as assay 5908, assay device 5910, detector 5920, logic system 5930, local memory 5935, external memory 5940, storage device 5945, and/or processor 5950. Process 1800 may include aspects described with process 1700.

At block 1810, a plurality of sequence reads of the cell-free DNA fragments is received. The plurality of sequence reads include ending sequences corresponding to ends of the plurality of cell-free DNA fragments. Block 1810 may be performed in a similar manner as block 1710.

At block 1820, a group of sequence reads located in one or more genomic regions is identified. Each of the one or more genomic regions has a histone modification associated with a target tissue type. Block 1820 may be performed in a similar manner as block 1720.

At block 1830, one or more sequence motifs corresponding to one or more ending sequences of a corresponding cell-free DNA fragment are determined for each sequence read of the group of sequence reads. Block 1830 may be performed in a similar manner as block 1730.

At block 1840, one or more relative frequencies of a set of the one or more sequence motifs are determined. The set of the one or more sequence motifs occurs at a higher rate in chromatin immunoprecipitation followed by sequencing (ChIP-seq) for the histone modification associated with the one or more genomic regions than in sequencing without chromatin immunoprecipitation. Block 1840 may be performed in a similar manner as block 1740.

At block 1850, an aggregate value of the one or more relative frequencies is determined. Block 1850 may be performed in a similar manner as block 1750.

At block 1860, the aggregate value is compared to one or more calibration values. The one or more calibration values are determined from one or more calibration samples whose values for the characteristic of the target tissue type are known. The comparison may be performed using a machine learning model, which may be any machine learning model described herein. The calibration values may be determined using the machine learning model.

The one or more calibration values may be determined in the same manner as block 1760, but using calibration samples whose values for the characteristic of the target tissue type are known. For example, the aggregate value determined from the biological sample may be a first aggregate value determined from one or more first relative frequencies. One or more second relative frequencies of the set of the one or more sequence motifs in the one or more genomic regions may be determined for each calibration sample of the one or more calibration samples. A second aggregate value may be determined for the one or more second relative frequencies for each calibration sample of the one or more calibration samples. Each of the one or more second aggregate values may thereby be associated with a value of the characteristic of the calibration samples. The calibration value may include the one or more second aggregate values. For example, the calibration values may be points along a line or a curve relating known values of the characteristic with the second aggregate values.

At block 1870, a first value for a characteristic of the target tissue type is estimated using the comparison. The first value for the characteristic may be the known first value associated with the calibration value, which may have a value close to or equal to the aggregate value. In some embodiments, the first value for the characteristic may be determined from a function or a line with the one or more calibration values. The function or line may relate known first values to the one or more calibration values.

The target tissue type may be liver or hematopoietic cells. The target tissue type may be fetal tissue. In some embodiments, the biological sample may be obtained from a pregnant female subject, and the target tissue type may be placental tissue. In some embodiments, the target tissue type may be an organ that has cancer. The target tissue type may be any organ described herein. The characteristic may be a level of cancer or a nutrition status of an organ. For example, the nutrition status of the organ may be if the organ is healthy or not, including any intermediate levels measuring health of the organ. As another example, the characteristic may be gestational age. In another example, the determined characteristic can be the concentration of a particular tissue type (e.g., liver cells) relative to the concentration of the other tissue type (e.g., hematopoietic cells).

In some embodiments, process 1800 may include using size frequencies along with relative frequencies of sequence motifs. Process 1800 may include measuring sizes of the cell-free DNA fragments using the sequence reads. Process 1800 may further include determining one or more size frequencies of the sequence reads for one or more size ranges, which may be any size range described herein. An aggregate value for the one or more size frequencies may be determined. The aggregate value may be a sum of size frequencies or any value analogous to the aggregate value for the relative frequencies of sequence motifs. In some embodiments, the aggregate value may be an estimation of the histone modifications. The levels of the histone modifications can be determined by various types of data, e.g., amounts of end motifs or fragment sizes. The aggregate value for the one or more size frequencies may be compared to calibration values that are determined with calibration samples whose values for the characteristic of the target tissue type are known. Estimating the first value for the characteristic may include using the comparison of the aggregate value for size frequencies, Similar to the comparison of the aggregative value for relative frequencies of sequence motifs.

Process 1800 may include additional implementations, such as any single implementation or any combination of implementations described herein and/or in connection with one or more other processes described herein.

C. Measuring Amount of Histone Modification

Sequence motifs may be used to determine the amount of a histone modification. As shown with FIGS. 14, 15A, and 15B, motif frequencies may be correlated with the cfChIP-seq signal associated with the H3K4me3 signal, which is proportional to the amount of H3K4me3. Hence, the motif frequency may be correlated with the amount of the histone modification. In addition, the amounts of histone modifications in different regions can be used to determine fractional concentrations of multiple tissues in the same sample.

1. Example Method for Determining Amount of Histone Modification Using Sequence Motifs

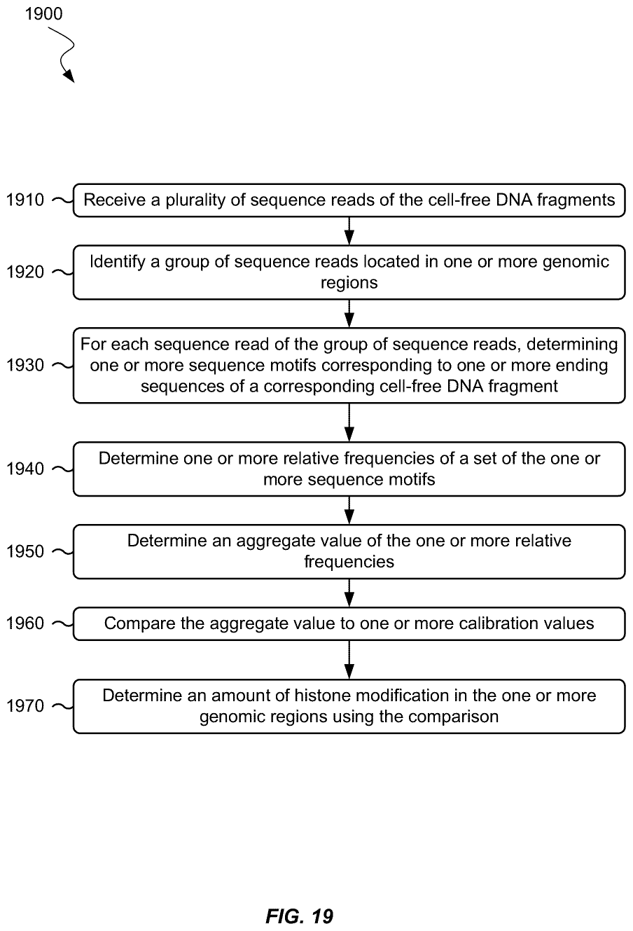

FIG. 19 is a flowchart of an example process 1900 associated with determining an amount of histone modification in one or more genomic regions. In some implementations, one or more process blocks of FIG. 19 may be performed by a system (e.g., measurement system 5900). In some implementations, one or more process blocks of FIG. 19 may be performed by another device or a group of devices separate from or including the system. Additionally, or alternatively, one or more process blocks of FIG. 19 may be performed by one or more components of measurement system 5900, such as assay 5908, assay device 5910, detector 5920, logic system 5930, local memory 5935, external memory 5940, storage device 5945, and/or processor 5950. Process 1900 may include aspects described with process 1700.

At block 1910, a plurality of sequence reads of the cell-free DNA fragments is received. The plurality of sequence reads include ending sequences corresponding to ends of the plurality of cell-free DNA fragments. Block 1910 may be performed in a similar manner as block 1710.

At block 1920, a group of sequence reads located in one or more genomic regions is identified. Each of the one or more genomic regions has a histone modification associated with a target tissue type. Block 1920 may be performed in a similar manner as block 1720.

At block 1930, one or more sequence motifs corresponding to one or more ending sequences of a corresponding cell-free DNA fragment are determined for each sequence read of the group of sequence reads. Block 1930 may be performed in a similar manner as block 1730.

At block 1940, one or more relative frequencies of a set of the one or more sequence motifs are determined. The set of the one or more sequence motifs occurs at a higher rate or a lower rate in chromatin immunoprecipitation followed by sequencing (ChIP-seq) for the histone modification associated with the one or more genomic regions than in sequencing without chromatin immunoprecipitation. Block 1940 may be performed in a similar manner as block 1740.

At block 1950, an aggregate value of the one or more relative frequencies is determined. Block 1950 may be performed in a similar manner as block 1750.

At block 1960, the aggregate value is compared to one or more calibration values. The one or more calibration values are determined from one or more calibration samples whose amounts of histone modifications are known. The amounts of histone modification in the one or more calibration samples may be known from performing ChIP-sequencing on each of the one or more calibration samples.

The one or more calibration values may be determined in the same manner as block 1760 or block 1860 but using calibration samples whose amounts of histone modifications are known. For example, the aggregate value determined from the biological sample may be a first aggregate value determined from one or more first relative frequencies. One or more second relative frequencies of the set of the one or more sequence motifs in the one or more genomic regions may be determined for each calibration sample of the one or more calibration samples. A second aggregate value may be determined for the one or more second relative frequencies for each calibration sample of the one or more calibration samples. Each of the one or more second aggregate values may thereby be associated with an amount of the histone modification of the calibration samples. The calibration value may include the one or more second aggregate values. For example, the calibration values may be points along a line or a curve relating known values of the characteristic with the second aggregate values.

At block 1970, an amount of histone modification in the one or more genomic regions is determined using the comparison. The amount of histone modification may be the known amount with the calibration value, which may have a value close to or equal to the aggregate value. In some embodiments, the amount of the histone modification may be determined from a function or a line with the one or more calibration values. The function or line may relate known amounts of the histone modification to the one or more calibration values. The amount of histone modification may be in the target tissue type.

Process 1900 may include additional implementations, such as any single implementation or any combination of implementations described herein and/or in connection with one or more other processes described herein.

Although FIG. 19 shows example blocks of process 1900, in some implementations, process 1900 may include additional blocks, fewer blocks, different blocks, or differently arranged blocks than those depicted in FIG. 19. Additionally, or alternatively, two or more of the blocks of process 1900 may be performed in parallel.

2. Example Method Using Fragmentomic Features

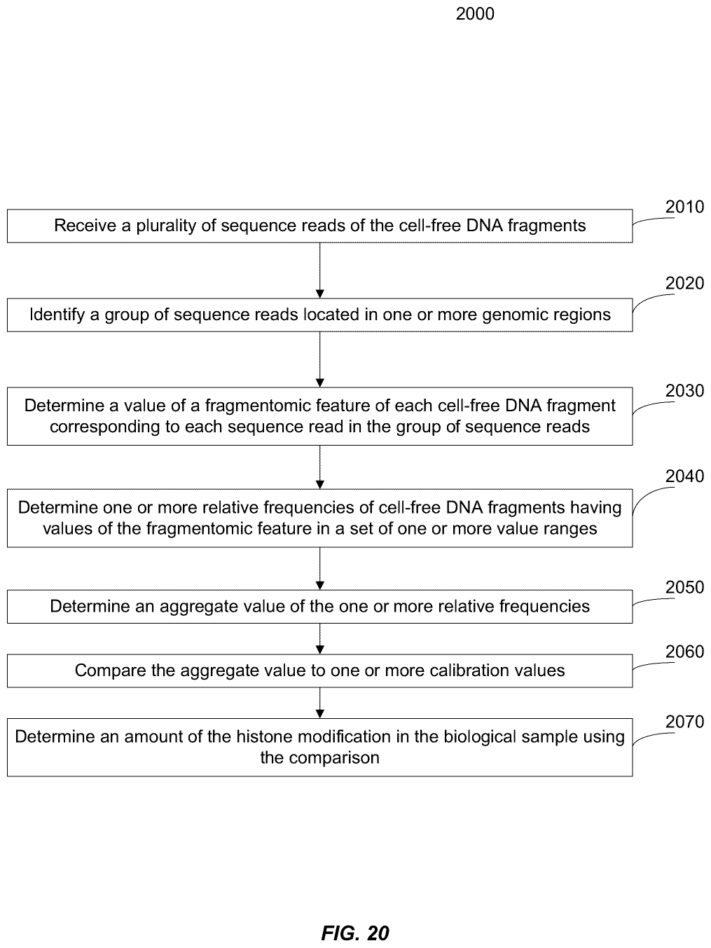

FIG. 20 is a flowchart of an example process 2000 associated with determining an amount of histone modification in one or more genomic regions. In some implementations, one or more process blocks of FIG. 20 may be performed by a system (e.g., measurement system 5900). In some implementations, one or more process blocks of FIG. 20 may be performed by another device or a group of devices separate from or including the system. Additionally, or alternatively, one or more process blocks of FIG. 20 may be performed by one or more components of measurement system 5900, such as assay 5908, assay device 5910, detector 5920, logic system 5930, local memory 5935, external memory 5940, storage device 5945, and/or processor 5950.

At block 2010, a plurality of sequence reads of the cell-free DNA fragments is received. Block 2010 may be performed in a similar manner as block 1710.

At block 2020, a group of sequence reads located in one or more genomic regions is identified. Each of the one or more genomic regions has a histone modification associated with a target tissue type. Block 2020 may be performed in a similar manner as block 1720.

At block 2030, a value of a fragmentomic feature of each cell-free DNA fragment corresponding to each sequence read in the group of sequence reads is determined. Fragmentomic feature may include fragment size, end motif, jagged-end (overhangs of one strand over the other), end nucleotide, topological form, and/or nucleosomal footprint. The fragmentomic feature may be any fragmentomic feature described herein.

For example, as described with FIG. 19, the fragmentomic feature may be the sequence motif corresponding to an ending sequence of an end of the cell-free DNA fragment, and the one or more value ranges are one or more sequence motifs.

As another example, the fragmentomic feature may be a size, and the one or more value ranges are one or more size ranges, as described in section IV.E.

As an example, the fragmentomic feature may be the topological form, and the one or more value ranges are one or more topological forms. The topological form may be circular or linear.

As an example, the fragmentomic feature is the nucleosomal footprint, and the one or more value ranges are one or more nucleosomal footprints. The nucleosomal footprint represents the binding pattern of the nucleosome to genomic DNA. The spaces between nucleosomes can be a value of the nucleosomal footprint.

At block 2040, one or more relative frequencies of cell-free DNA fragments having values of the fragmentomic feature in a set of one or more value ranges are determined. The set of the one or more value ranges occurs at a differential rate in chromatin immunoprecipitation followed by sequencing (ChIP-seq) for the histone modification associated with the one or more genomic regions than in sequencing without chromatin immunoprecipitation. The differential rate may be higher or lower and may be by a statistically significant amount. Block 2040 may be performed in a similar manner as block 1740 but using one or more value ranges of the fragmentomic feature instead of the one or more sequence motifs. In other embodiments, the set of the one or more value ranges determined by sequencing samples without cell-free chromatin immunoprecipitation are determined by focusing on genomic regions containing differential rates with higher or lower histone modification signals predetermined from other reference samples or databases.

At block 2050, an aggregate value of the one or more relative frequencies is determined. The aggregate value may be a sum of the one or more relative frequencies or a statistical measure (e.g., mean, median, mode, percentile) of the one or more relative frequencies.

At block 2060, the aggregate value is compared to one or more calibration values. The one or more calibration values are determined from one or more calibration samples whose amounts of histone modifications are known. The amounts of histone modification in the one or more calibration samples may be known from performing cfChIP-sequencing on each of the one or more calibration samples. The one or more calibration values may be determined in the same manner as block 1960 but using frequencies of one or more value ranges of a fragmentomic feature instead of one or more sequence motifs.

At block 2070, an amount of the histone modification in the biological sample is determined using the comparison. The amount of histone modification may be in the target tissue type. Block 2070 may be performed in a similar manner as block 1970.

The amount of histone modification may be used to determine a fractional concentration of a target tissue, a classification of a level of a disorder, or a classification of a transplant status of a target tissue type (e.g., as described with process 2000).

Although FIG. 20 shows example blocks of process 2000, in some implementations, process 2000 may include additional blocks, fewer blocks, different blocks, or differently arranged blocks than those depicted in FIG. 20. Additionally, or alternatively, two or more of the blocks of process 2000 may be performed in parallel.

3. Determining Fractional Concentrations Using Deconvolution

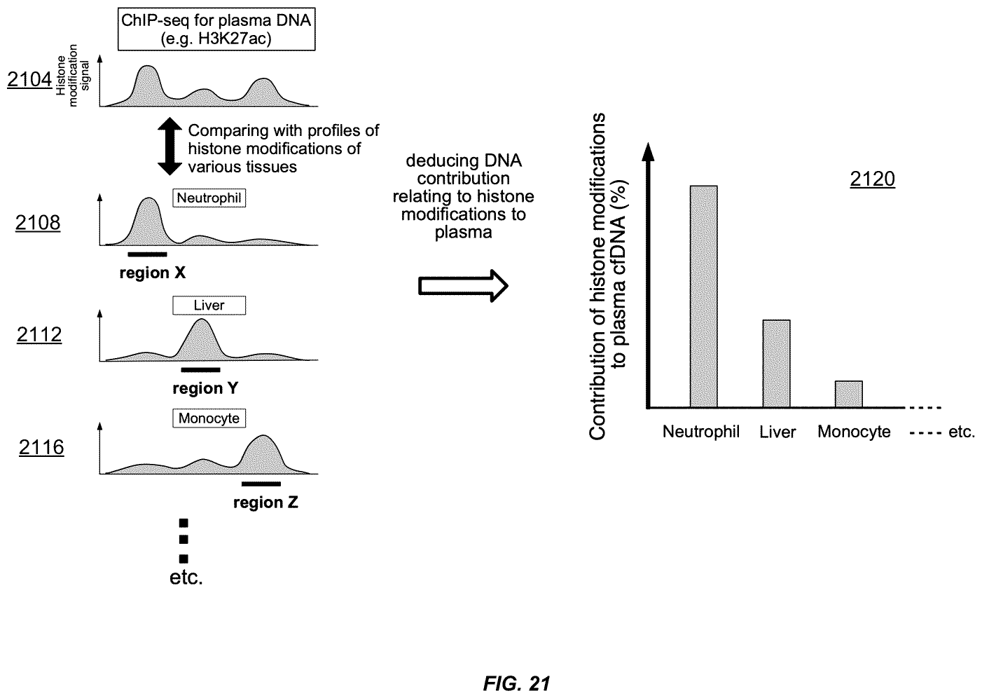

The fractional concentrations of multiple tissue types can be determined through a deconvolution process. FIG. 21 shows applying ChIP-seq to determine the contribution from different tissues. Graph 2104 is a graph of the histone modification signal from ChIP-seq on the y-axis and genomic position on the x-axis. Graphs 2108, 2112, and 2116 show tissue-specific regions for histone modifications signals. Graph 2108 shows that region X carries neutrophil-specific histone modifications. Graph 2112 shows that region Y carries liver-specific histone modifications. Graph 2116 shows that region Z carries monocyte-specific histone modifications.

The plasma DNA ChIP signals across those informative genomic regions were compared with the patterns of ChIP signals across different tissues, deducing the proportional DNA contributions related to H3K27ac into plasma from different tissues. Graph 2120 shows the deduced proportional DNA contribution of different tissues.

Based on FIG. 21, a biological sample including DNA from multiple tissues can have H3K4me3 cfChIP-seq signals in the same region(s) from multiple tissues. For example, the genomic region X represented in graph 2108 has neutrophils with the highest H3K4me3 signals but lower signals in the other tissues (e.g., liver and monocyte). Similarly, the genomic region Y represented in graph 2112 also has different signals across different tissues, including the neutrophils, liver, and monocyte. The genomic region Z represented in graph 2116 also has different signals across different tissues, including the neutrophils, liver, and monocyte. The overlapping H3K4me3 signals in the same regions can allow for fractional concentrations of tissues to be determined.

A system of linear equations, one for each region, can be solved to determine the fractional concentrations for each tissue in a cell-free mixture, such as a plasma sample.

$$\begin{matrix}\begin{matrix}\begin{matrix}{H}_{A} = {f}_{1} {h}_{1 , A} + {f}_{2} {h}_{2 , A} + … + {f}_{n} {h}_{n , A} \\ {H}_{B} = {f}_{1} {h}_{1 , B} + {f}_{2} {h}_{2 , B} + … + {f}_{n} {h}_{n , B}\end{matrix} \\ ⋮\end{matrix} \\ {H}_{m} = {f}_{1} {h}_{1 , m} + {f}_{2} {h}_{2 , m} + … + {f}_{n} {h}_{n , m}\end{matrix}$$

The set of linear equations is for m genomic regions and n tissues. H_{A} represents the total histone modification amount in genomic region A in the sample, as may be measured using one or more sequence motifs. H_{B} represents the total histone modification amount in genomic region B. H_{A} and H_{B} may represent the same or different histone modifications. Hₘ represents the total histone modification amount in genomic region m. The fractional concentration for target tissue 1 is f₁, for target tissue 2 is f₂, and for target tissue n is fₙ. Target tissue 1 is known to have an amount h_{1,A} in genomic region A, an amount h_{1,B} in genomic region B, and an amount h_{1,m} in genomic region m. Target tissue 2 is known to have an amount h_{2,A} in genomic region A, an amount h_{2,B} in genomic region B, and an amount h_{2,m} in genomic region m. Target tissue n is known to have an amount h_{n,A} in genomic region A, an amount h_{n,B} in genomic region B, and an amount h_{n,m} in genomic region m. In some embodiments, the matrix H may represent the histone modification amounts as measured using one or more sequence motifs. H and h may not need to be directly calculated to solve for fractional concentrations if there are appropriate sequence motif amounts to use.

The amounts of histone modifications in target tissues in certain genomic regions (e.g., h_{1,A}, h_{1,B}, etc.) may be relative amounts. These amounts may be determined from a calibration sample. For instance, a calibration sample having half target tissue 1 and half target tissue 2 may show certain ratio of histone modification amounts, and that ratio can be used for h_{1,A} and h_{1,B}.

The number of equations should be more or equal to the number of target tissues in order to solve for the fractional concentrations. The number of equations can equal the number of genomic regions and therefore the number of genomic regions can equal the number of target tissues. If the sum of the fractional concentrations is known (e.g., sum is 1), then the number of genomic regions can equal the number of regions minus 1. With the histone modification amounts in each genomic region measured through using sequence motifs, the fractional concentrations can be determined by solving the system of equations.

Accordingly, in some embodiments, multiple tissue types may have the same or similar sequence motifs associated with histone modifications in the same genomic regions. The fractional concentration of each of these multiple tissue types can be determined through a deconvolution process. The deconvolution process may include solving a set of linear or nonlinear equations, such as the ones described herein.

The amount of histone modification may be determined as described with process 1900. In process 1900, the group of sequence reads is a first group of sequence reads. The one or more genomic regions are one or more first genomic regions. The set of the one or more sequence motifs is a set of one or more first sequence motifs. The one or more relative frequencies are one or more first relative frequencies. The aggregate value is a first aggregate value. The one or more calibration values are one or more first calibration values. The amount of histone modification is a first amount of histone modification. An example of the first amount is H_{A} in the equations described above.

A second amount of histone modification in one or more second genomic regions may be determined for the system of linear equations. An example of the second amount is H_{B}. The histone modification may be associated with a first tissue type and the second tissue type in the one or more first genomic regions.

The histone modification may be associated with the first tissue type and the second tissue type in one or more second genomic regions. For example, the one or more first genomic regions may be regions associated with region X in FIG. 21 and the one or more second genomic regions may be regions associated with region Y. As another example, the one or more first genomic regions and the one or more second genomic regions may be regions within the same box (e.g., region X or region Y).

A second group of sequence reads located in the one or more second genomic regions is identified. The identification may be performed in a similar manner as described with block 1920. Each of the one or more second genomic regions may have the histone modification associated with the first tissue type and the second tissue type. In some embodiments, the histone modification in the one or more second genomic regions may have a histone modification that is different from the one in the one or more first genomic regions.

For each sequence read of the second group of sequence reads, one or more second sequence motifs corresponding to one or more ending sequences of a corresponding cell-free DNA fragment are determined. The determination may be performed in a similar manner as described with block 1930.

One or more second relative frequencies of a set of the one or more second sequence motifs are determined. The set of the one or more second sequence motifs occurs at a higher rate in ChIP-seq for the histone modification associated with the one or more second genomic regions than in sequencing without chromatin immunoprecipitation. The determination may be performed in a similar manner as described with block 1940.

A second aggregate value of the one or more second relative frequencies is determined. The determination may be performed in a similar manner as described with block 1950.

The second aggregate value is compared to one or more second calibration values. The comparison may be performed in a similar manner as block 1960.

The second amount of histone modification in the one or more second genomic regions is determined using the comparison. The determination may be performed in a similar manner as block 1970.

The first fractional concentration of the first tissue type and the second fractional concentration of the second tissue type is determined by solving a system of linear or nonlinear equations. The system of linear equations may be the set of equations described herein. The system of linear equations may include the first amount of histone modification (e.g., H_{A}), the second amount of histone modification (e.g., H_{B}), and parameters specifying relative amounts of the respective histone modification for each tissue type in the one or more first genomic regions and the one or more second genomic regions (e.g., h_{1,A}, h_{1,B}, h_{2,A}, h_{2,B}). The first fractional concentration may be f₁, and the second fractional concentration may be f₂.

Biological samples may include more than two target tissue types. Methods for determining the fractional concentrations of two target tissue types can be extended for three or more tissue types.

In embodiments, the histone modification may be associated with a third tissue type in the one or more first genomic regions and the one or more second genomic regions. The histone modification may be associated with the first tissue type, the second tissue type, and the third tissue type in one or more third genomic regions. The process may involve performing similar steps as described for the second tissue type. The process may include determining a third amount of histone modification (e.g., Hₘ where m is C) in the one or more third genomic regions in the same manner as determining the second amount of histone modification. The third fractional concentration of the third tissue type may be determined by solving the system of linear or nonlinear equations. The system of linear equations may include the third amount of histone modification and parameters for relative amounts for each tissue type in the one or more third genomic regions.

D. Classifying Level of Disorder

Sequence motifs may be used to classify a level of a disorder. The disorder may be specific to a particular tissue type or may apply to the subject. Sequence motifs may indicate an amount or presence of a histone modification, and that amount or presence of a histone modification may be associated with a particular level of disorder. The amount or presence of the histone modification, however, may not need to be determined in order to use the sequence motifs to classify a level of a disorder.

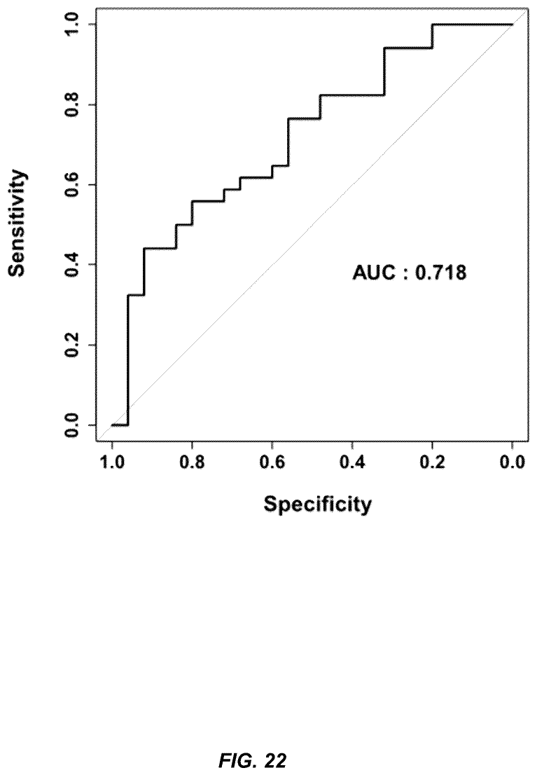

FIG. 22 is an ROC curve for differentiating patients with and without hepatocellular carcinoma (HCC) using the deduced H3K4me3 signals in liver-specific H3K4me3 regions using end motifs. Specificity is shown on the x-axis, and sensitivity is shown on the y-axis. Using plasma H3K4me3 ChIP signals deduced by end motifs had an AUC of 0.718 for differentiating between patients with and without HCC using a cutoff. These results show that ChIP signals of histone modifications deduced by end motifs would be clinically useful for non-invasive prenatal testing and cancer detection and monitoring.

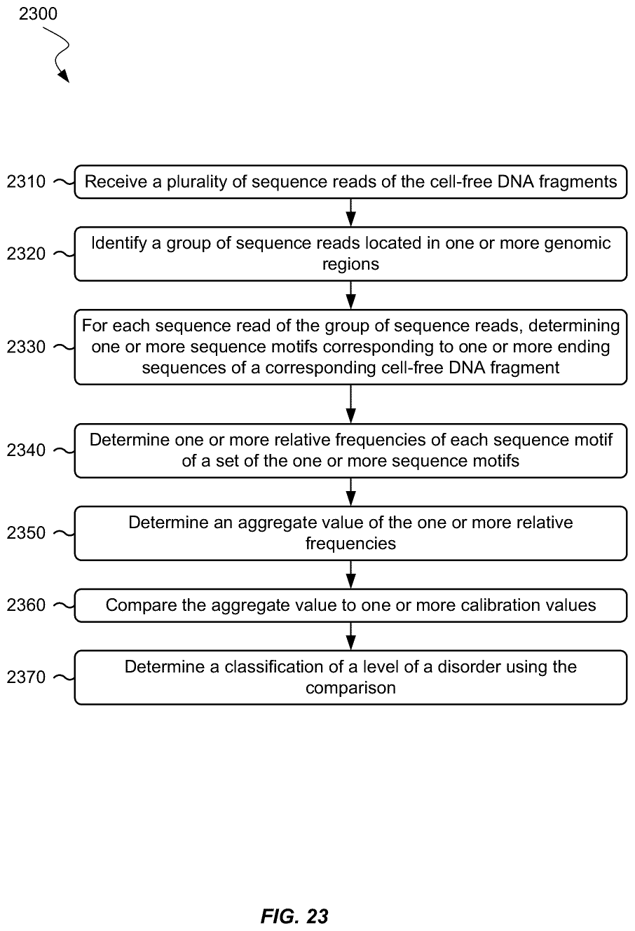

FIG. 23 is a flowchart of an example process 2300 associated with classifying a level of a disorder. In some implementations, one or more process blocks of FIG. 23 may be performed by a system (e.g., measurement system 5900). In some implementations, one or more process blocks of FIG. 23 may be performed by another device or a group of devices separate from or including the system. Additionally, or alternatively, one or more process blocks of FIG. 23 may be performed by one or more components of measurement system 5900, such as assay 5908, assay device 5910, detector 5920, logic system 5930, local memory 5935, external memory 5940, storage device 5945, and/or processor 5950. Process 2300 may include aspects described with process 1700.

At block 2310, a plurality of sequence reads of the cell-free DNA fragments is received. The plurality of sequence reads include ending sequences corresponding to ends of the plurality of cell-free DNA fragments. Block 2310 may be performed in a similar manner as block 1710.

At block 2320, a group of sequence reads located in one or more genomic regions is identified. Each of the one or more genomic regions has a histone modification associated with one or more target tissue types. Block 2320 may be performed in a similar manner as block 1720.

At block 2330, one or more sequence motifs corresponding to one or more ending sequences of a corresponding cell-free DNA fragment are determined for each sequence read of the group of sequence reads. Block 2330 may be performed in a similar manner as block 1730.

At block 2340, one or more relative frequencies of a set of the one or more sequence motifs are determined. The set of the one or more sequence motifs occurs at a higher rate in chromatin immunoprecipitation followed by sequencing (ChIP-seq) for the histone modification associated with the one or more genomic regions than in sequencing without chromatin immunoprecipitation. Block 2340 may be performed in a similar manner as block 1740.

At block 2350, an aggregate value of the one or more relative frequencies is determined. Block 2350 may be performed in a similar manner as block 1750.

At block 2360, the aggregate value is compared to one or more calibration values. The one or more calibration values are determined from one or more calibration samples whose classifications of the level of a disorder are known.

The one or more calibration values may be determined in the same manner as block 1760, block 1860, or block 1960, but using calibration samples whose classifications of the level of the disorder are known. For example, the aggregate value determined from the biological sample may be a first aggregate value determined from one or more first relative frequencies. One or more second relative frequencies of the set of the one or more sequence motifs in the one or more genomic regions may be determined for each calibration sample of the one or more calibration samples. A second aggregate value may be determined for the one or more second relative frequencies for each calibration sample of the one or more calibration samples. Each of the one or more second aggregate values may thereby be associated with a classification of the level of the disorder. The calibration value may include the one or more second aggregate values. For example, the calibration values may be points along a line or a curve relating known classifications of the level of the disorder with the second aggregate values.

At block 2370, a classification of a level of a disorder is determined using the comparison. The classification of the level of the disorder may be the known classification with the calibration value, which may have a value close to or equal to the aggregate value. In some embodiments, the classification of the level of the disorder may be determined from a function or a line with the one or more calibration values. The function or line may relate known classifications to the one or more calibration values. In some embodiments, the classification may be a level of an abnormality.

The disorder may be in the target tissue type. The disorder may be cancer of the target tissue type. The cancer may include hepatocellular carcinoma (HCC), colorectal cancer (CRC), or any cancer described herein. In some embodiments, the disorder is a pregnancy-associated disorder. The disorder may be a blood disorder. The disorder may be any disorder described herein.

In embodiments, process 2300 may include using size frequencies, as described with process 1800.

Process 2300 may include additional implementations, such as any single implementation or any combination of implementations described herein and/or in connection with one or more other processes described herein.

Although FIG. 23 shows example blocks of process 2300, in some implementations, process 2300 may include additional blocks, fewer blocks, different blocks, or differently arranged blocks than those depicted in FIG. 23. Additionally, or alternatively, two or more of the blocks of process 2300 may be performed in parallel.

### IV. Deducing Histone Modifications Using Size Information

A. Size Information for Deducing ChIP Signal

Plasma DNA size information can be used for detecting and quantifying histone modifications present in plasma DNA molecules. Like the relationship between cfDNA end motifs information and histone modification level, size information of cfDNA molecule may be influenced by histone modification level, i.e., epigenetic status. We analyzed the size information of cfDNA molecules within regions of interest. Those regions involving histone modifications can be grouped into different categories according to the magnitudes of ChIP signal. For example, the regions were first sorted according to the magnitudes of ChIP signal and then were empirically classified into 9 categories (e.g., FIG. 4). After obtaining region categories with different H3K27ac ChIP signals, the DNA size information of plasma DNA sequencing results without immunoprecipitation in different region categories can be compared.

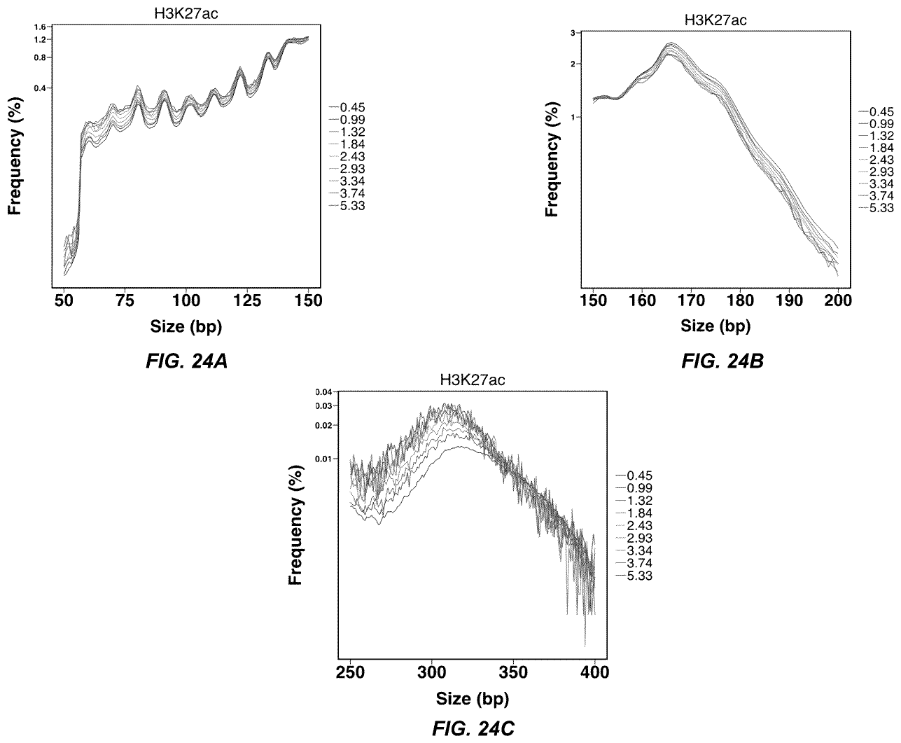

FIGS. 24A, 24B, and 24C show percentages of cfDNA molecules with certain sizes in the region categories for different levels of H3K27ac signal. The x-axis is the size in base pairs from plasma DNA sequencing without H3K27ac-based precipitation. The y-axis is the percentage of cfDNA molecules having the size. The different color lines in each graph show the different region categories using H3K27ac ChIP signals. FIG. 24A shows a size range from 50 to 140 bp. FIG. 24B shows a size range from 150 to 200 bp. FIG. 24C shows a size range from 250 to 350 bp. As shown in FIGS. 24A-24C, the size profiles change according to the strength of ChIP signals. For example, the higher the ChIP signal, the more the DNA molecules within a range of 270 to 300 bp would be observed. Additionally, the size difference showed different trends for different size ranges. For example, for sizes from about 165 to 200 bp, the higher the ChIP signal, the fewer the DNA molecules. For sizes from about 60 to 100 bp, the higher the ChIP signal, the more the DNA molecules. Thus, it is feasible to deduce plasma DNA histone modifications based on size information of plasma DNA molecule without immunoprecipitation.

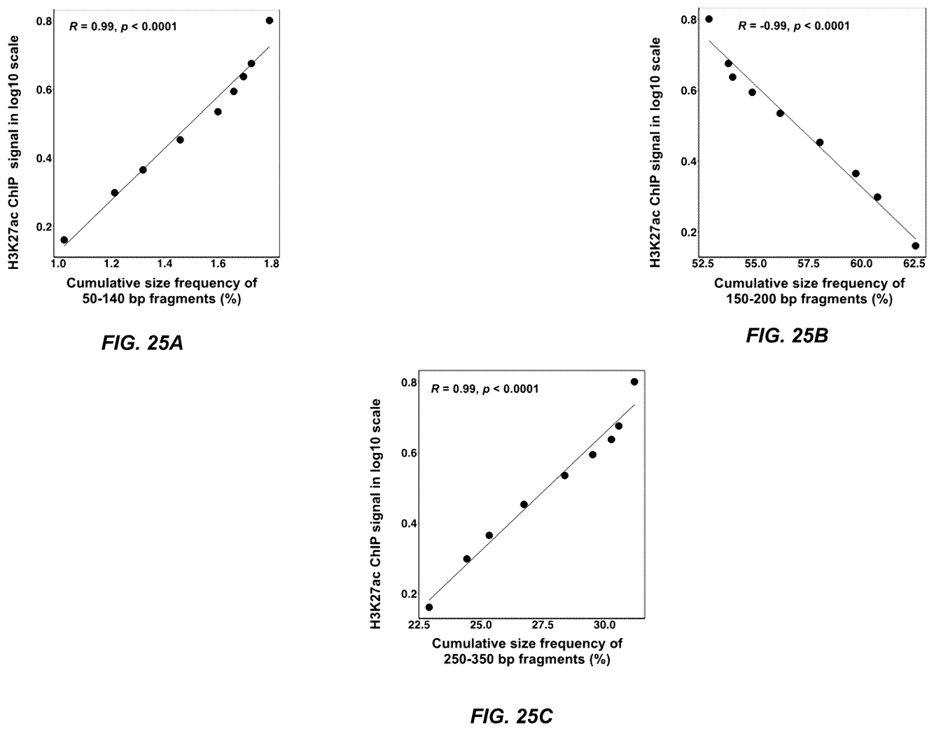

FIGS. 25A, 25B, and 25C show that the correlation between sizes and ChIP signals of histone modification can be generalized to other histone modifications (e.g., H3K27ac). The x-axis is the cumulative size frequency of certain size fragments as a percentage for plasma DNA without immunoprecipitation. The y-axis is the H3K27ac ChIP signal on a log₁₀ scale. FIG. 25A is for 50-140 bp fragments. FIG. 25B is for 150-200 bp fragments. FIG. 25C is for 250-350 bp fragments. Across the 9 categories, for a plasma DNA without immunoprecipitation, the percentages of cfDNA molecules within a size range of 50-140 bp and 250-350 bp were positively correlated with the log-transformed ChIP signals obtained from ChIP-seq data, with a Pearson's r of 0.99 (P value: <0.0001) (FIG. 25A) and 0.99 (P value: <0.0001) (FIG. 25C). The percentages of cfDNA molecules within a size range of 150-200 bp were negatively correlated with the log-transformed ChIP signals (Pearson's r: −0.99; P value: <0.0001) (FIG. 25B).

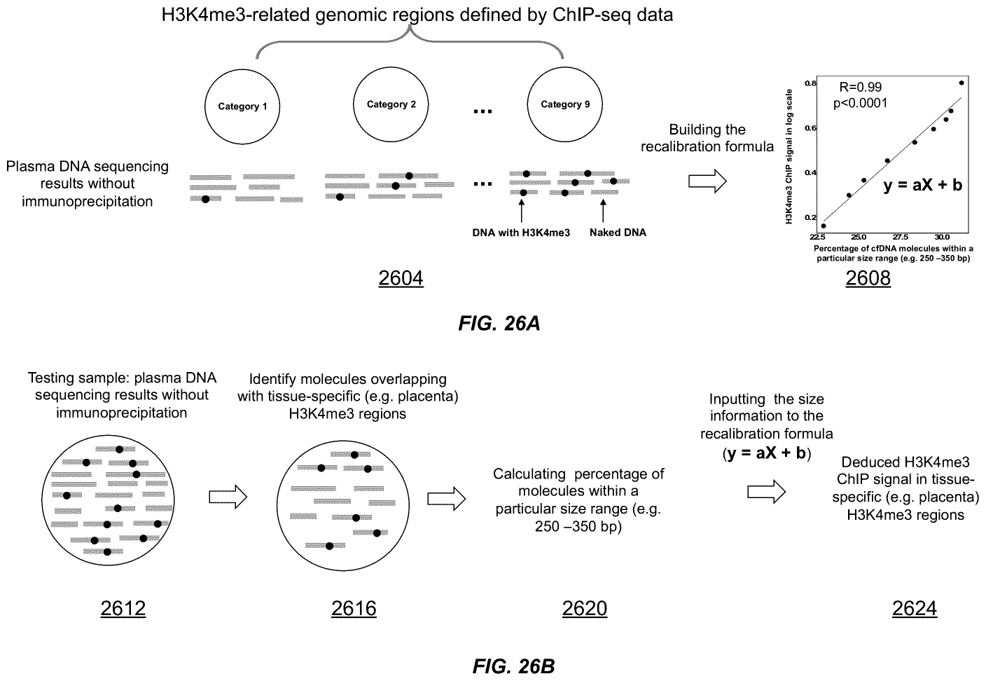

FIG. 26A and FIG. 26B illustrate the use of size information to deduce plasma DNA histone modifications for plasma DNA sequencing results without immunoprecipitation. FIG. 26A shows building the recalibration formula with the percentage of a certain size range of cfDNA molecules and the level of H3K4me3 ChIP signals in the 9 categories. Stage 2604 shows regions involving H3K4me3 were grouped into different categories according to the magnitudes of ChIP signal. As illustrated in FIG. 26A, the size information (e.g., percentages of cfDNA molecules from plasma DNA sequencing results without immunoprecipitation within a size range of 250-350 bp) originating from each region category could be used to determine the correlation with the H3K4me3 ChIP signals. In stage 2608, based on the correlation between fragment sizes and ChIP signals (on a log scale), a recalibration formula can be determined. A linear formula is shown as an example of a recalibration formula, but non-linear formulas may also be used.

FIG. 26B shows how the recalibration formula can be used to infer the ChIP signals in other regions (e.g., placenta-specific H3K4me3 regions) according to the corresponding size information of those regions (i.e., deduced ChIP signal). At stage 2612, plasma DNA are sequenced without immunoprecipitation. At stage 2616, molecules overlapping with tissue-specific (e.g., placenta) H3K4me3 regions are identified. At stage 2620, percentages of molecules within a particular size range (e.g., 250-350 bp) in H3K4me3-based immunoprecipitated plasma DNA are calculated. The size information is inputted into the recalibration formula, and at stage 2624, the H3K4me3 ChIP signal is deduced in tissue-specific (e.g., placenta) H3K4me3 regions.

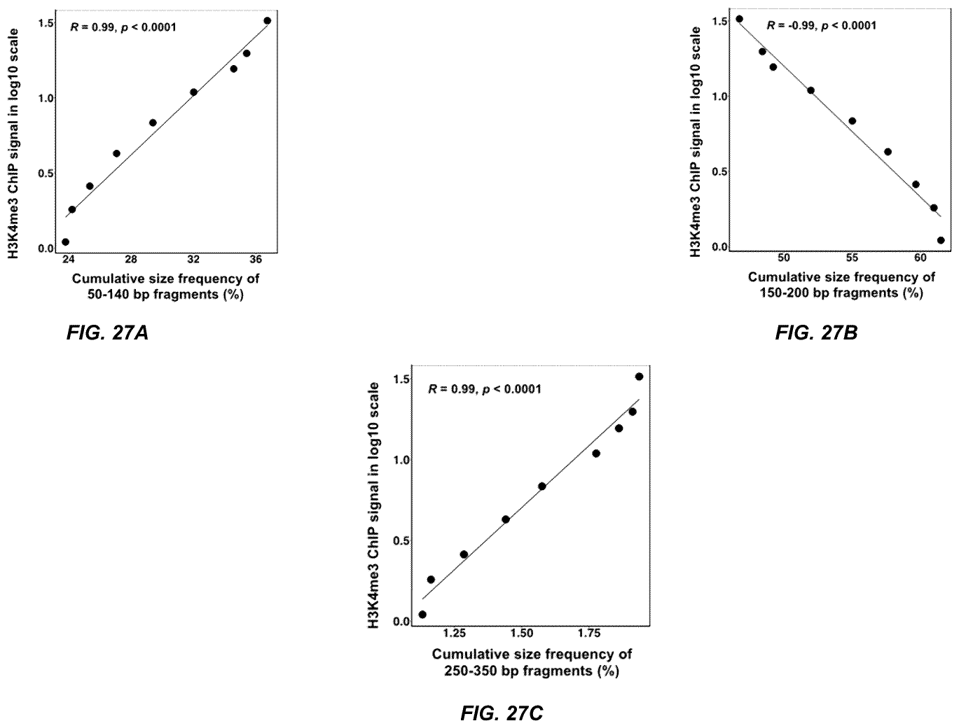

FIGS. 27A, 27B, and 27C show the correlation between the percentage of cfDNA molecules within a size range and the log-transformed H3K4me3 ChIP signal. The x-axis is the cumulative size frequency of certain size fragments as a percentage for plasma DNA without immunoprecipitation. The y-axis is the H3K4me3 ChIP signal on a log₁₀ scale. FIG. 27A is for 50-140 bp fragments. FIG. 27B is for 150-200 bp fragments. FIG. 27C is for 250-350 bp fragments. Across those 9 categories, for a plasma DNA without immunoprecipitation, the percentages of cfDNA molecules within a size range of 50-140 bp and 250-350 bp were positively correlated with the log-transformed ChIP signals obtained from ChIP-seq data, with a Pearson's r of 0.99 (P value: <0.0001) (FIG. 27A) and 0.99 (P value: <0.0001) (FIG. 27C). The percentages of cfDNA molecules within a size range of 150-200 bp were negatively correlated with the log-transformed ChIP signals (Pearson's r: −0.99; P value: <0.0001) (FIG. 27B). The results show fragment size patterns can be used to deduce histone modifications in plasma DNA molecules (referred to be as deduced ChIP signals).

B. Deduced ChIP Signals and Fetal Fraction

We further used a linear regression model to build a model (i.e., recalibration formula) for deducing the H3K4me3 ChIP signal in a region of interest or in a set of regions of interest. As an example, we trained a model for each sample for deducing the ChIP signals based on a size range of 250-350 bp, namely Y=aX+b where ‘Y’ represented the log-transformed ChIP signal, ‘X’ represented the percentage of cfDNA molecules within a size range of 250-350 bp from a particular genomic region of interest or a set of regions of interest for which histone modifications were to be determined. ‘a’ and were the slope and intercept, respectively. In one embodiment, we determined the percentage of cfDNA molecules within a size range of 250-350 bp from those placental-specific regions in terms of H3K4me3. We analyzed 30 plasma DNA samples of pregnant women. The size range of 250-350 bp was chosen for illustrative purposes. Other size ranges may also be used. Size ranges can be selected using a machine learning model.

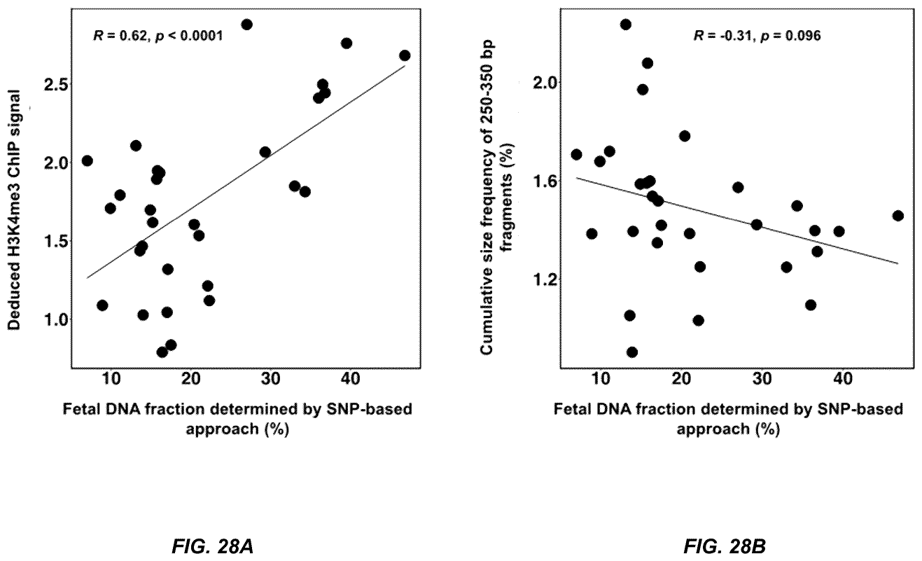

FIG. 28A and FIG. 28B show evaluating the performance of deduced H3K4me3 ChIP signals in placenta-specific H3K4me3 regions for fetal DNA fraction deduction. The x-axis shows the fetal DNA fraction as a percent as determined by an SNP-based approach. In FIG. 28A, the y-axis is the deduced H3K4me3 ChIP signal using a size range of 250-350 bp. Using the size metric, the deduced H3K4me3 ChIP signals correlated with fetal DNA fraction (Pearson's r: 0.62; P value: <0.0001).

In FIG. 28B, the y-axis is the cumulative size frequency of 250-350 bp fragments as a percentage. There was no significant correlation between the percentage of plasma DNA within a size range of 250-350 bp (Pearson's r: −0.31, P value: 0.096). These results in FIGS. 28A and 28B show that the use of deduced ChIP signals for plasma DNA samples without immunoprecipitation can allow for analyzing the tissues of origin for plasma DNA molecules.

We further used a linear regression model to build a model (i.e., recalibration formula) for deducing the H3K27ac ChIP signal in a region of interest or in a set of regions of interest. As an example, we trained a model for each sample for deducing the ChIP signals based on a size range of 250-350 bp, namely Y=aX+b where ‘Y’ represents the log-transformed ChIP signal, ‘X’ represents the percentage of cfDNA molecules within a size range of 250-350 bp from a particular genomic region of interest or a set of regions of interest for which histone modifications were to be determined. ‘a’ and ‘b’ represent the slope and intercept, respectively. In one embodiment, we determined the percentage of cfDNA molecules within a size range of 250-350 bp from those placental-specific regions in terms of H3K27ac. We analyzed 30 plasma DNA samples of pregnant women.

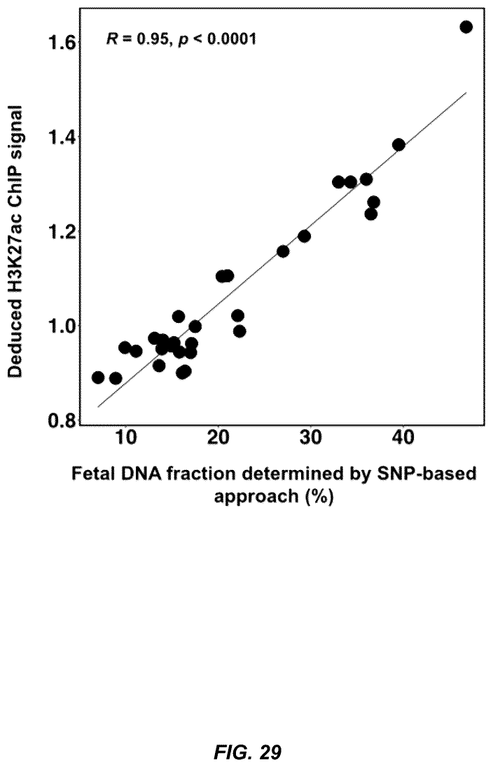

FIG. 29 is a graph evaluating the performance of deduced H3K27ac ChIP signal in placenta-specific H3K27ac regions for determining fetal DNA fraction. The x-axis is the fetal DNA fraction as a percent as determined by an SNP-based approach. The y-axis is the deduced H3K27ac ChIP signal using a size range of 250-350 bp. Based on such size metric, the deduced ChIP signals of H3K27ac showed a higher correlation with fetal DNA fraction (Pearson's r: P value: <0.0001), compared with H3K4me3 based analysis (Pearson's r: 0.62; P value: <0.0001) (FIG. 28A). These results highlighted that different types of histone modification can be used to determine the tissues of origin for plasma DNA molecules through ChIP signals of histone modification deduced by cfDNA size information.

We analyzed different size ranges for deducing the H3K27ac ChIP signals and correlated the deduced H3K27ac ChIP signals with the tissue DNA fraction determined by SNP-based approach. We analyzed 30 plasma DNA samples of pregnant women. The size ranges of bp, 160-225 bp, and 230-350 bp were used for illustrative purposes. Other size ranges may also be used in some other embodiments.

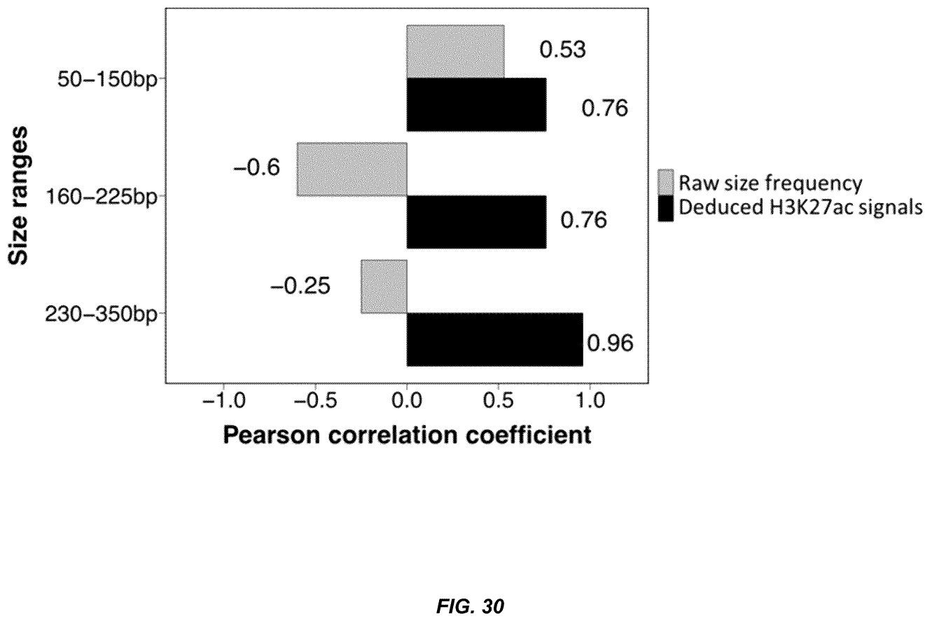

FIG. 30 is a graph showing how well correlated size ranges considering and not considering histone modification levels are to the fetal DNA fraction. The y-axis shows the three size ranges tested. The x-axis shows the Pearson correlation coefficient. For each size range, two different bars are shown. The top bar (gray color) in each pair shows the Pearson correlation coefficient for using the raw size frequency. The bottom bar (black color) in each pair shows the Pearson correlation coefficient for the deduced H3K27ac signal levels in placenta-specific H3K27ac regions.

As shown in FIG. 30, the fetal DNA fractions determined by SNP-based approach were strongly correlated with the deduced H3K27ac signal levels in the placenta-specific H3K27ac regions with a size range of 230-350 bp (Pearson's r: 0.96; P value: <0.0001). By contrast, no such correlation was observed with the raw cumulative size frequency per se (Pearson's r: −0.25; P value=0.18). Comparisons were also performed for other size ranges. For all the tested size ranges, the deduced H3K27ac levels in the placenta-specific H3K27ac regions showed a substantially higher correlation with the fetal DNA fraction (Pearson's r: 0.76 to 0.96), compared to the respective raw cumulative size frequency (Pearson's r: −0.25 to 0.53). In addition, the deduced H3K27ac ChIP signals based on molecules with the size range of 230-350 bp showed the best performance (Pearson's r=0.96) compared to the other tested size ranges (Pearson's r: 0.76).

C. Deduced ChIP Signals and Cancer

In one embodiment, we explored whether the deduced ChIP signal of histone modification from plasma DNA without immunoprecipitation would be informative for cancer detection. We analyzed 34 patients with hepatocellular carcinoma (HCC), 17 subjects with chronic hepatitis B virus (HBV) and 8 healthy control samples.

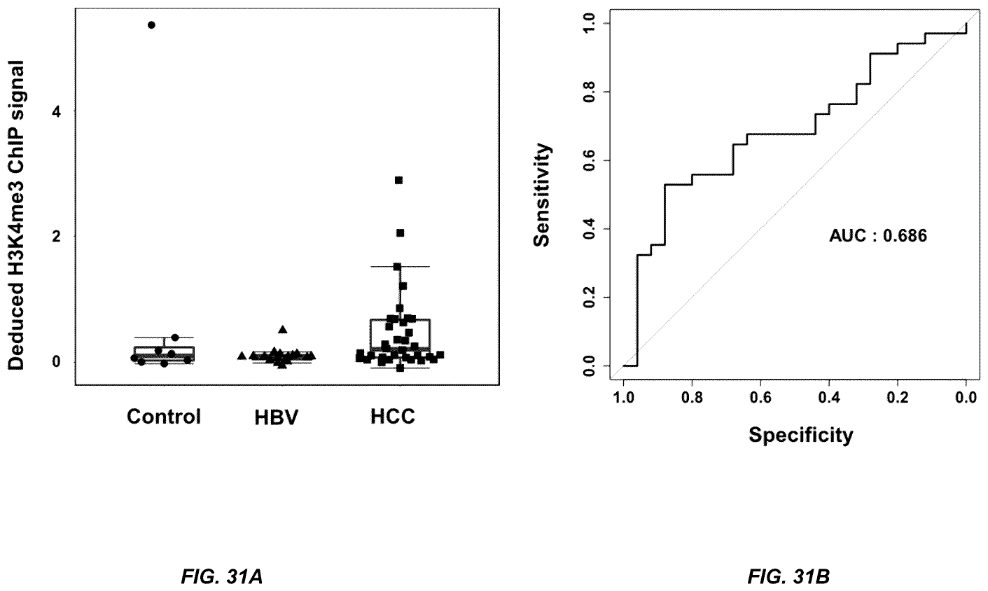

FIG. 31A and FIG. 31B are graphs showing using deduced H3K4me3 ChIP signals based on liver-specific H3K4me3 regions for HCC detection. The H3K4me3 ChIP signals were deduced using the cumulative frequency of molecules within a size range of 250 to 350 bp. FIG. 31A shows box plots of the deduced H3K4me3 ChIP signal (y-axis) versus the subject type (x-axis). For liver-specific regions, the deduced H3K4me3 ChIP signals was significantly higher in subjects with HCC (median: 0.21; range: 0-2.90), compared with subjects without HCC (median: 0.09; range: 0-5.36) (P value: 0.015, Mann-Whitney U test).

FIG. 31B is a receiver operating characteristic (ROC) curve. ROC analysis revealed that one could achieve an AUC of 0.686 in differentiating subjects with and without HCC cancer. These results show that deduced ChIP signals can be used for cancer detection. This approach would obviate the need of an immunoprecipitation assay prior to sequencing, thus reducing the cost and experimental time and making it readily incorporated with other technologies such as whole-genome random or targeted sequencing, or whole-genome random or targeted bisulfite sequencing.

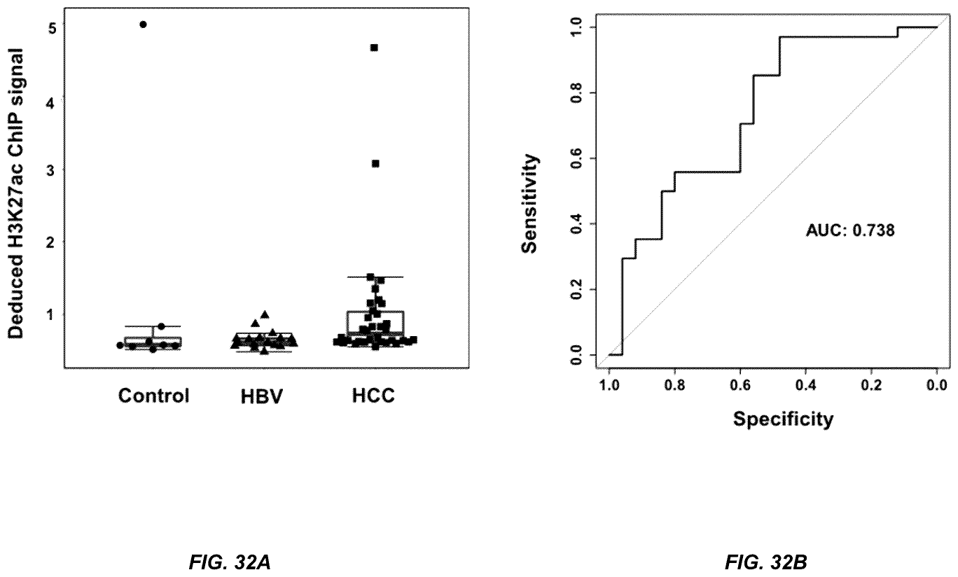

FIG. 32A and FIG. 32B show using deduced H3K27ac ChIP signals based on H3K27ac regions for HCC detection. The H3K27ac ChIP signals were deduced using the cumulative frequency of molecules within a size range of 250 to 350 bp. FIG. 32A and FIG. 32B are the same as FIG. 31A and FIG. 31B, respectively, except for using H3K27ac regions instead of H3K4me3 regions. The use of deduced ChIP signals related to H3K27ac improved classification power when discriminating patients with and without HCC, increasing the AUC to (FIG. 32B) from 0.686 (FIG. 31B).

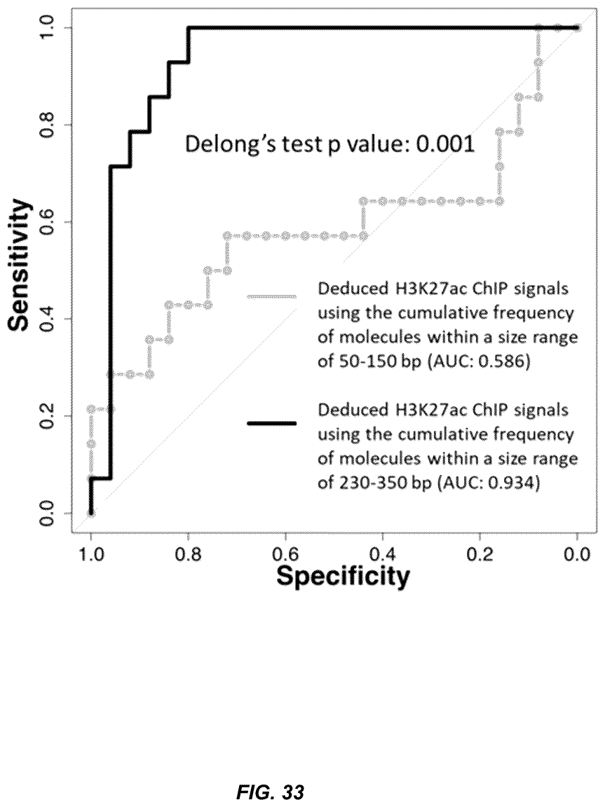

FIG. 33 is a graph showing how size selection affects performance for differentiating patients with cancer from healthy controls. FIG. 33 is an ROC curve with sensitivity on the y-axis and specificity on the x-axis. The ROC curve is for differentiating subjects with hepatocellular carcinoma (HCC) at intermediate and advanced stages from subjects without HCC by deduced H3K27ac ChIP signals for liver-specific regions. The black line is for molecules within a size range of 230-350 bp. The gray line is for molecules within a size range of 50-150 bp.

The ROC analysis revealed that the deduced H3K27ac ChIP signal using the cumulative frequency of molecules within a size range of 230-350 bp in liver-specific H3K27ac regions achieved a significantly higher area under the receiver operating characteristic curve (AUC) of 0.934 for differentiating patients with HCC at the intermediate and advanced stages from patients without HCC, compared to that within a size range of 50-150 bp (AUC: 0.586) (P=0.001; Delong's test).

D. Deduced ChIP Signals and Transplants

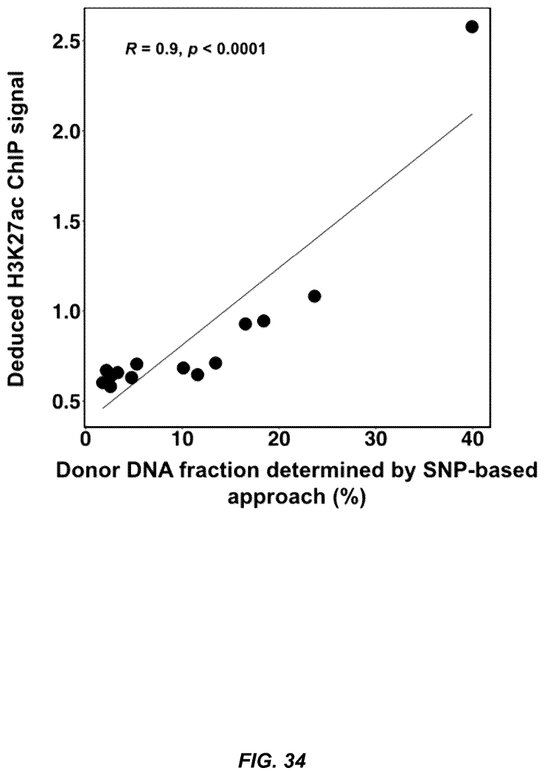

FIG. 34 is a graph showing correlation between deduced H3K27ac ChIP signals in liver-specific H3K27ac regions and donor DNA fraction. The H3K27ac ChIP signals were deduced using the cumulative frequency of molecules within a size range of 250 to 350 bp. The y-axis shows the deduced H3K27ac ChIP signal. The x-axis shows the donor DNA fraction as a percent. We deduced the H3K27ac ChIP signal of in plasma DNA of patients with liver transplantation using liver-specific regions. The graph shows a high correlation between the liver contributions determined by deduced ChIP signals of histone modifications in liver-specific regions according to the embodiments in this disclosure and donor DNA fraction by SNP-based approach (Pearson's r: 0.9; P value: <0.0001). The data shows deduced H3K27ac ChIP signals for liver-specific regions can allow for monitoring the subjects with organ transplantation.

We further analyzed plasma DNA sequencing results without immunoprecipitation for a cohort of 14 liver transplantation patients. The size ranges of 50-150 bp, 160-225 bp, and 230-350 bp were used for illustrative purposes. Other size ranges may also be used in some other embodiments.

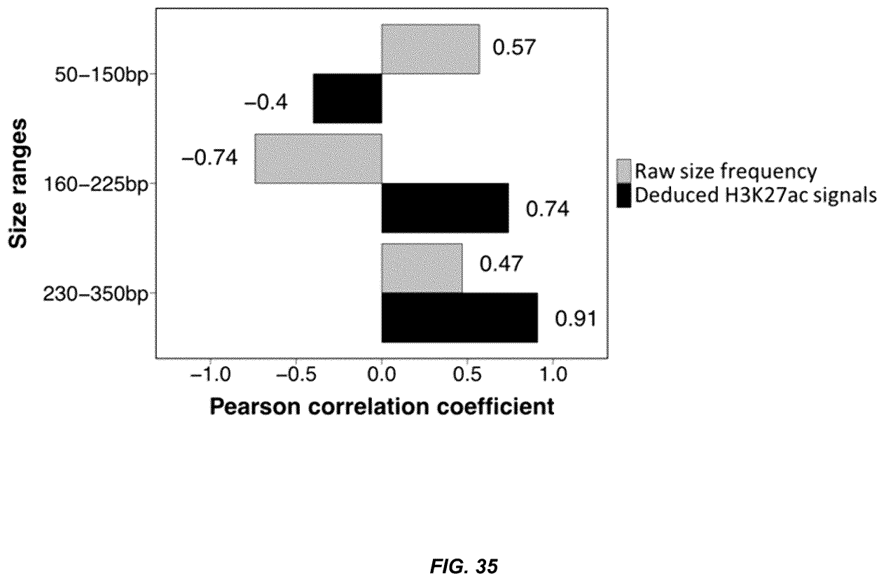

FIG. 35 is a graph showing how well correlated size ranges considering and not considering histone modification levels are to the donor DNA fraction determined by SNP-based approach. The y-axis shows the three size ranges tested. The x-axis shows the Pearson correlation coefficient. For each size range, two different bars are shown. The top bar (gray color) in each pair shows the Pearson correlation coefficient for using the raw size frequency. The bottom bar (black color) in each pair shows the Pearson correlation coefficient for the deduced H3K27ac signal levels in liver-specific H3K27ac regions.

As shown in FIG. 35, the highest correlation was observed between the donor DNA fraction and the deduced H3K27ac value (Pearson's r: 0.91; P value: <0.0001) in the liver-specific H3K27ac regions by those molecules with the size range of 230-350 bp.

E. Example Method for Determining Histone Modification Using Sizes

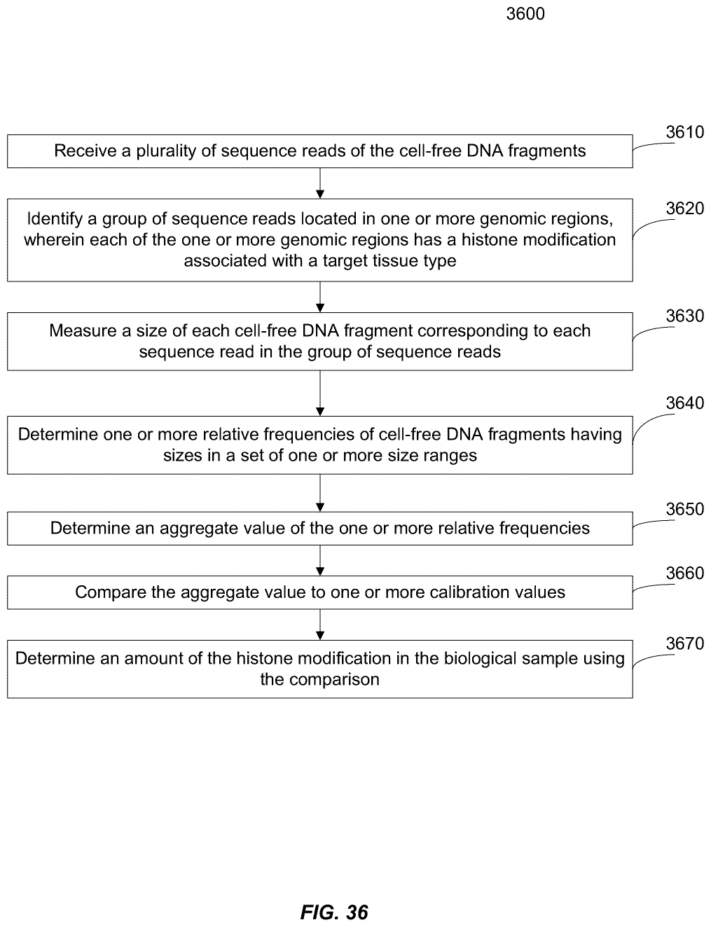

FIG. 36 is a flowchart of an example process 3600 associated with determining an amount of histone modification in one or more genomic regions using fragment sizes. In some implementations, one or more process blocks of FIG. 36 may be performed by a system (e.g., measurement system 5900). In some implementations, one or more process blocks of FIG. 36 may be performed by another device or a group of devices separate from or including the system. Additionally, or alternatively, one or more process blocks of FIG. 36 may be performed by one or more components of measurement system 5900, such as assay 5908, assay device 5910, detector 5920, logic system 5930, local memory 5935, external memory 5940, storage device 5945, and/or processor 5950.

At block 3610, a plurality of sequence reads of the cell-free DNA fragments is received. The plurality of sequence reads may be obtained by random massively parallel sequencing. The plurality of sequence reads may be obtained using paired-end sequencing.

At block 3620, a group of sequence reads located in one or more genomic regions is identified. Each of the one or more genomic regions has a histone modification associated with one or more target tissue types. Block 3620 may be performed in a similar manner as block 1720.

At block 3630, a size of each cell-free DNA fragment corresponding to each sequence read in the group of sequence reads is measured. The size of a fragment can be measured using paired-end sequencing, aligning the sequence to a genome, and then deducing the size from the genome coordinates of the aligned sequences. In some embodiments, the size of a fragment may be measured by sequencing the entire fragment and then determining the size from the sequence.

At block 3640, one or more relative frequencies of cell-free DNA fragments having sizes in a set of one or more size ranges are determined. The set of the one or more size ranges may occur at a differential rate in chromatin immunoprecipitation followed by sequencing (ChIP-seq) for the histone modification associated with the one or more genomic regions than in sequencing without chromatin immunoprecipitation. The differential rate may be higher or lower and may be by a statistically significant amount. The one or more size ranges may include 50 to 100 bp, 100 to 150 bp, 150 to 200 bp, 200 to 250 bp, 250 to 300 bp, 300 to 350 bp, 350 to 400 bp, 400 to 450 bp, 450 to 500 bp, over 500 bp, or any combination thereof.

At block 3650, an aggregate value of the one or more relative frequencies is determined. The aggregate value may be a sum of the one or more relative frequencies or a statistical measure (e.g., mean, median, mode, percentile) of the one or more relative frequencies.

At block 3660, the aggregate value is compared to one or more calibration values. The one or more calibration values are determined from one or more calibration samples whose amounts of histone modifications are known. The amounts of histone modification in the one or more calibration samples may be known from performing cfChIP-sequencing on each of the one or more calibration samples. The one or more calibration values may be determined in the same manner as block 1960 but using frequencies of one or more size ranges instead of one or more sequence motifs.

At block 3670, an amount of the histone modification in the biological sample is determined using the comparison. The amount of histone modification may be in the target tissue type. Block 3670 may be performed in a similar manner as block 1970.

The amount of histone modification may be used to determine a fractional concentration of a target tissue, a classification of a level of a disorder, or a classification of a transplant status of a target tissue type. The amount of histone modification can be determined using sequence motifs, fragmentomic features, or any other technique, in addition to size ranges.

In some embodiments, the amount of the histone modification may be compared to one or more second calibration values. The one or more second calibration values may be determined from one or more second calibration samples whose fractional concentrations of a target tissue type and amounts of histone modification are known. A fractional concentration of the target tissue type may be determined using the comparison of the amount of the histone modification to the one or more second calibration values.

In some embodiments, the amount of the histone modification may be compared to one or more third calibration values. The one or more third calibration values may be determined from one or more third calibration samples whose level of a disorder and amounts of histone modification are known. A classification of a level of a disorder is determined using the one or more third calibration values. The disorder may be any disorder described herein.

In some embodiments, the amount of the histone modification is compared to one or more fourth calibration values. The one or more fourth calibration values may be determined from one or more fourth calibration samples whose transplant status and amounts of histone modification are known. A classification of a transplant status of the target tissue type is determined using the one or more fourth calibration values. Classifications of a transplant status include whether the transplanted organ is rejected by the subject.

Although FIG. 36 shows example blocks of process 3600, in some implementations, process 3600 may include additional blocks, fewer blocks, different blocks, or differently arranged blocks than those depicted in FIG. 36. Additionally, or alternatively, two or more of the blocks of process 3600 may be performed in parallel.

V. Tissue Contributions Deduced from Histone Modifications

The characteristic size profile of cfDNA shows a modal frequency at approximately 166 bp, with smaller molecules forming a series of peaks in a 10-bp periodicity (Lo et al. Sci Transl Med. 2010; 2:61ra91). Such size patterns of plasma DNA fragments suggest the presence of histone proteins bound to cfDNA molecules. One recent study revealed the presence of histone modifications associated with cfDNA molecules in plasma, using cell-free chromatin immunoprecipitation followed by sequencing (cfChIP-seq) (Sadeh et al. Nat Biotechnol. 2021; 39:586-598). However, Sadeh et al's study did not provide any approach for deducing the percentage contribution of chromatin modifications from various tissues/organs.

Sadeh et al. analyzed the average number of reads per kilobase across genomic regions associated with a tissue-specific histone modification of a tissue as a signal to indicate the contribution from that tissue. The tissue-specific regions deduced from reference tissues were considered as independent factors when analyzing those signals (Sadeh et al. 2021). One limitation of the method described by Sadeh et al. is that when a tissue lacks the tissue-specific histone modifications or the number of regions showing tissue-specific histone modifications is not sufficient in a tissue, the DNA contribution from the tissue cannot be accurately deduced. The method in Sadeh relies on the absolute signals of histone modifications in plasma regarding a tissue-specific region. However, the relative strength of the signals of histone modifications in each reference tissue was not taken into account in this approach by Sadeh et al., likely leading to the inaccurate analysis or no analysis.

For example, the reads per kilobase in a genomic region related to a histone modification for a tissue may be governed by at least two factors: the first factor is the percentage of DNA (including DNA not related to a histone modification) contributed by such a tissue, and the second factor is the level of histone modification present in that tissue. The analysis adjusted by the level of histone modification present in that tissue is important for the tissue contribution analysis based on histone modifications. Sadeh et al. attempted to analyze percentage contribution from the liver using linear regression. The plasma DNA of healthy subjects was considered to have 0% liver contribution, and DNA from liver tissue was considered to have 100% liver contribution. The differences in histone modifications between the liver tissues and plasma DNA of healthy subjects were used to determine the liver contribution in other plasma DNA samples (Sadeh et al. Nat Biotechnol. 2021). Such an analysis did not use histone modification signals from two or more tissues. Plasma DNA includes contributions from various tissues, and the liver contributions to plasma may vary across healthy subjects. Thus, the assumption for linear regression analysis may not hold true under the circumstances.

Hence, the contributions from two or more tissues being analyzed cannot be accurately deduced in Sadeh et al.'s approach. The strength of histone modification signal from each tissue is important in quantitatively analyzing signals present in plasma cfDNA. The strength of histone modification signal may refer to the percentage of cells harboring the histone modification of interest in a tissue, which can be measured by the depth of sequencing read coverage present in ChIP-seq. The approaches, by not using the signals of histone modifications across different tissues, would greatly deteriorate the performance in determining the contributions of cfDNA with histone modifications into plasma from different tissues.

In this disclosure, we developed approaches of comparing the relative signals of histone modifications plasma DNA with the signals from reference tissues to deduce the percentage contribution from each cell type or tissue, herein referred to as plasma DNA tissue mapping by histone modifications. In one embodiment, such comparison would consider the signals of modified histone from various tissues as covariates to deconvolute the percentage contributions from various tissues to plasma, for example, but not limited to, using quadratic programming, non-negative least squares (NNLS), etc. Sun et al. demonstrated that comparing methylation signals of plasma DNA with methylation signals of various tissues allowed deduction of the percentage contributions of DNA molecules into plasma across tissues through the use of quadratic programming (Sun et al., Proc Natl Acad Sci USA. 2018; 115:E5106). However, the histone modification would occur at amino acid sequences of histone proteins, where the signal properties of modified signal are distinct from DNA methylation. The procedures of signal processes in DNA methylation analysis could not be used for modified histones. Histone modifications involve post-translational modification of a histone protein, which impacts their interactions with DNA. By contrast, the DNA methylation is a biochemical process where a DNA base, usually cytosine, is enzymatically methylated at the 5-carbon position. Histone modification and methylations involve different types of biochemical machinery. In some embodiments of the disclosure, one could deduce the contribution of histone modification into plasma through comparing the number of DNA immunoprecipitated via one or more antibodies of interest with the counterpart measures across various reference tissues. In contrast to the approach used by Sadeh et al.'s study in which only the tissue-specific histone modifications were informative, the approach present in this disclosure could make use of both tissue-specific histone modifications and tissue-variable histone modifications.

A. Plasma DNA Tissue Mapping by Histone Modifications

In embodiments, the percentage contribution of DNA into plasma from various cell types could be determined by comparing the profile of plasma DNA histone modifications with profiles of histone modifications derived from a number of organs, tissues, or cells. For example, one could apply H3K27ac ChIP-seq to a number of tissues including, but not limited to, neutrophils, megakaryocytes, T cells, B cells, erythrocytes, monocytes, natural killer cells, or cells from the liver, colon, adipose tissues, brain, pancreas, placenta, heart, lung, kidney, spleen, bladder, stomach, etc. One could determine informative genomic regions carrying tissue-specific histone modifications (e.g., H3K27ac). An informative genomic region refers to a region that preferentially enriched a certain histone modification (e.g., H3K27ac) in a particular tissue (e.g., the liver) but was relatively depleted of such modification in other tissues. Such regions could be referred to tissue-specific histone modification regions (e.g., tissue-specific H3K27ac regions). In some embodiments, an informative genomic region referred to a region that showed variable signals of certain histone modification (e.g., H3K27ac) across tissues of interest. The variable signals could be defined by the coefficient of variation (CV) of the histone signal that exceeded but not limited to 5%, 10%, 20%, 30%, 40%, 50%, 60%, 70%, 80%, 90%, 100%, 200%, etc. and the difference in modified histone signal between maximum and minimum exceeded a certain cutoff, such as but not limited 1, 2, 3, 4, 5, 6, 7, 8, 9, 10, 50, 100, 500, 1000, 5000, 10,000 reads per kilobase, etc. Such regions can be defined as tissue-variable histone modification regions (e.g., tissue-variable H3K27ac regions). FIG. 3, as described previously, shows applying ChIP-seq to determine the contribution from different tissues.

As different pathological or physiological states would alter chromatin status in certain cell types, we conjectured that the analysis of histone modifications of cfDNA molecules would allow noninvasive detection and monitoring of diseases, for example, fetal abnormalities in pregnant women, cancer, autoimmune diseases, the presence of transplant rejection, blood disorders, etc.

B. Examples of Plasma DNA Tissue Mapping by Histone Modifications

Deduced histone modification signals can be used to determine fetal DNA fraction, to determine specific tissue contributions to the sample, to classify subjects as pregnant or non-pregnant, and to classify subjects with a likelihood of a disorder (e.g., cancer).

1. Biological Samples from Pregnant Females

We recruited 19 pregnant samples, with a median gestation age of 38 weeks. Plasma was isolated from whole blood within 6 hours of sample collection through sequential steps of centrifugation: centrifugation at 1,600 g for 10 minutes followed by re-centrifugation of the plasma portion at 16,000 g for another 10 minutes. Plasma could be stored at −80° C. We used two types of histone modifications (H3K27ac and H3K4me3) as examples. Antibody conjugated beads were incubated with plasma by rotating overnight at 4° C. and washing with wash buffer, and the immunoprecipitated DNA was ligated with barcoded adapters on beads. The DNA was eluted, followed by the amplification through PCR. DNA library was sequenced in multiplex sequencing together with several other libraries by the Illumina platform (e.g., Nextseq 500 or NovaSeq 6000), with a median of 4.30 million paired-end reads (range: 0.10-30.73). We performed H3K27ac ChIP-seq for 19 pregnant samples, 13 non-pregnant samples, and 12 samples with hematological diseases (10 beta-thalassemia major samples, 1 iron deficiency anemia sample, and 1 aplastic anemia sample). Moreover, we performed H3K4me3 ChIP-seq for 12 pregnant women, 4 non-pregnant healthy subjects and 4 patients with hematological diseases (2 with beta-thalassemia major, 1 with iron deficiency anemia, and 1 with aplastic anemia sample).

The fetal DNA fraction in maternal plasma for each pregnant woman was calculated based on a single nucleotide polymorphism (SNP)-based approach (Lo et al. Sci Transl Med. 2010; 2:61ra91). The genotypes regarding the maternal buffy coat and placental tissue samples were obtained using microarray-based genotyping technology (Illumina Infinium Omni 2.5-8 array), and informative SNPs were identified (i.e., where the mother was homozygous (denoted as AA genotype), and the fetus was heterozygous (denoted as AB genotype)). Fetal-specific DNA fragments were identified according to the DNA fragments carrying fetal-specific alleles at informative SNP sites. In this scenario, the B allele was fetal-specific, and the DNA fragments carrying the B allele were deduced to be originated from fetal tissues. The number of fetal-specific molecules (p) carrying the fetal-specific alleles (B) was determined. The number of molecules (q) carrying the shared alleles (A) was determined. The fetal DNA fraction across all cell-free DNA samples would be calculated by 2p/(p+q)*100%.

ChIP-seq data for various tissues were obtained from public databases for illustration purposes. The public databases used herein included, but not limited to, the Blueprint project (blueprint-epigenome.eu/), the ENCODE project (encodeproject.org/), and the Roadmap project (roadmapepigenomics.org/). In total, we obtained H3K27ac ChIP-seq results from 18 tissue types, including but not limited to neutrophils, monocytes, B cells, T cells, natural killer cells, erythroblast cells, and megakaryocytes, the liver, brain, pancreas, placenta, heart, colon, lung, adipose, kidney, spleen, and bladder), with a median of 22.5 million paired-end/single-end reads (range: 12-45 million). Additionally, we obtained H3K4me3 ChIP-seq data from 19 tissues, including but not limited to neutrophils, monocytes, B cells, T cells, natural killer cells, erythroblast cells, megakaryocytes, the liver, brain, pancreas, placenta, heart, colon, lung, adipose, kidney, spleen, bladder, and stomach, with a median of 25 million paired-end reads (range: 7-32 million).

Based on ChIP-seq data from various tissues, we determined informative genomic regions which carried tissue-specific histone modifications. In one embodiment, one could analyze a number of genomic regions that were known to be enriched in a particular type of histone modifications. For example, H3K4me3 was known to preferentially occur at regions nearby transcriptional start sites (i.e., promoter regions). Hence, one could determine ChIP signals across regions nearby a transcriptional start site (TSS). In one embodiment, the ChIP signal for a region of interest can be determined by the percentage of sequencing reads overlapping such a region among the total mapped reads. In another embodiment, the ChIP signal for a region of interest can be determined by the percentage of sequencing reads overlapping with such a region among the total mapped reads related to all regions of interest. The ChIP signals would be adjusted by GC biases and mapping biases, expressing as fragments per kilobase per million (i.e., FPKM) analyzed fragments.

In one embodiment, according to the ChIP signals identified from a number of tissues/organs, a human reference genome would be classified as regions with the presence of certain histone modifications (e.g., H3K27ac) (denoted as regions of interest [ROIs]), and regions with the absence of such said histone modifications (denoted as background regions). ChIP-seq reads of plasma DNA present in background regions might be due to non-specific antibody (Ab) binding during the experimental process, which was considered as background noise. The raw ChIP signal of an ROI was determined as the number of fragments for which the end fell within that ROI. In some embodiments, the raw ChIP signal of a ROI was determined as the number of fragments for which at least one or more nucleotides in a molecule overlapped with that ROI. The raw signal of a ROI can be deducted by the background noise across background regions surrounding such a ROI being interrogated.

Taking H3K27ac as an example, we divided the genome into non-overlapping 5-Mb windows. For each 5-Mb window, we calculated the raw signals in ROIs (N regions) that were bound by H3K27ac according to the ChIP results shown in the ENCODE and Blueprint projects. The remaining regions (M regions) were deemed background regions for determining the noise. Poisson distribution could be used for estimating the average sequence depth per one kilobase (kb) across M background regions, referred to as estimated background noise. The raw ChIP signals across N ROIs deducted by the estimated background noise (i.e., noise-deducted ChIP signals) would be used for the downstream analysis. To minimize the influence of sequencing depths on the comparison of ChIP signals between samples, we determined the scaling factors of sequencing depth across samples using sequencing reads from those regions that were shown to be bound by H3K27ac across various samples. The noise-deducted ChIP signals would be adjusted by the corresponding scaling factors of sequencing depth. In one embodiment, one could further express the ChIP signals aforementioned as fragments per kilobase per million (FPKM). In some embodiments, for the background noise estimation, a number of overlapping windows could be used. The window sizes could be, but not limited to, 10 kb, 50 kb 100 kb, 500 kb, 1 Mb, 2 Mb, 3 Mb, 4 Mb, 5 Mb, 10 Mb, etc.

The regions carrying tissue-specific histone modifications (i.e., tissue-specific regions) can be determined using the following criteria:
1. The first tissue of interest had the highest ChIP signal for each tissue-specific region across all the tissues being analyzed and its normalized ChIP signal was greater than 15.
2. The ratio of ChIP signal in the log 2 scale at a tissue-specific region between the first tissue and the second tissue that had the second-highest ChIP signal is greater than 3.
   As result, we identified a total of 4,245 tissue-specific regions for H3K27ac, and 807 tissue-specific regions for H3K4me3.

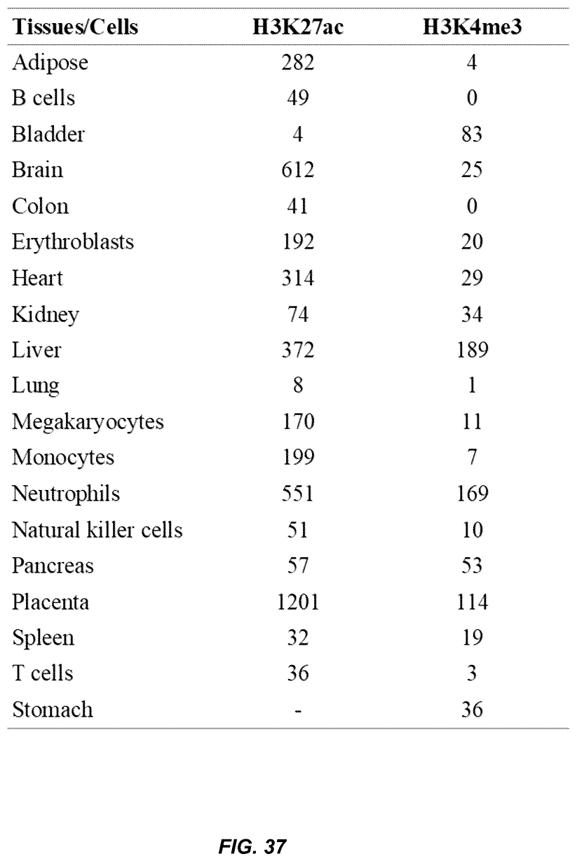

FIG. 37 shows a table of tissue-specific histone modification regions. The first column lists the tissue/cell type. The second column lists the number of regions for that tissue showing H3K27ac modification. The third column lists the number of regions for that tissue showing H3K4me3 modification.

In one embodiment, the selected regions were not necessarily restricted to tissue-specific regions. One could use region(s) showing a high variability in histone modification signals across the panel of tissues of interest for analysis (tissue-variable regions). These regions could be determined using the following criteria:
1. The first tissue of interest had the highest ChIP signal for each region being analyzed across all the tissues, with a normalized ChIP signal greater than 15. Normalization may consider background noise, sequencing depth, GC bias, length of ROI, as explained above. The normalized ChIP signal may be expressed as fragments per kilobase per million (i.e., FPKM).
2. The relative percentage difference between the highest (denoted as H) and lowest (denoted as L) ChIP signals across all the tissue types was required to be at least 20% (i.e., (H-L)/L*100%≥20%).
3. The coefficient of variation (CV) of the ChIP signal across all the tissue types was required to be at least 25%, where CV was defined as the ratio of the standard deviation to the mean times 100%.
   As result, we identified a total of 27,941 tissue-variable regions for H3K27ac, and 17,321 tissue-variable regions for H3K4me3.

For plasma ChIP-seq data of H3K27ac, the number of plasma DNA fragments with their 5′ end overlapping each tissue-specific region of H3K27ac was determined. The normalized ChIP signal in FPKM was calculated for each tissue-specific region accordingly. Similarly, for plasma DNA ChIP data of H3K4me3, the number of plasma DNA fragments with their 5′ end overlapping each tissue-specific region of H3K4me3 was determined. The normalized ChIP signal was calculated for each tissue-specific region accordingly. Comparing the ChIP signals of plasma DNA to ChIP signals from various tissues allowed us to deduce the DNA contribution into the plasma DNA pool that is related to histone modifications of interest.

In one embodiment, the measured ChIP signal levels of DNA molecules were recorded in a vector (X) and the retrieved reference ChIP signal levels across different tissues were recorded in a matrix (M). The proportional contributions (P) from different tissues to plasma DNA pool were deduced by quadratic programming:

X̅ᵢ=Σₖ(pₖ×Mᵢₖ),

where X̅ᵢ represents a ChIP signal level of a tissue-specific or tissue-variable region i in the plasma DNA mixture; pₖ represents the proportional contribution of concerned histone medications of a cell type k to the plasma DNA mixture; Mᵢₖ represents the ChIP signal level of the tissue-specific or tissue-variable region i in the cell type k. When the number of regions was the same or larger than the number of cell types, the values of individual pₖ could be determined.

The aggregated DNA contribution related to a particular type of histone modifications from all cell types would be constrained to be 100%:

Σₖpₖ=100%,

Furthermore, any contribution from a cell type would be required to be non-negative:

pₖ≥0,∀k

Hence, pₖ could be deduced by, but not limited to, quadratic programming with a program written in Python (python.org) or R language (r-project.org). In some other embodiments, one could use, but not limited to, linear or non-linear regression, non-negative least squares, Bayesian framework, etc. In some embodiments the regions used for tissue contribution deduction could be tissue-specific regions only, or tissue-variable regions only, or the combination of both tissue-specific and tissue-variable regions.

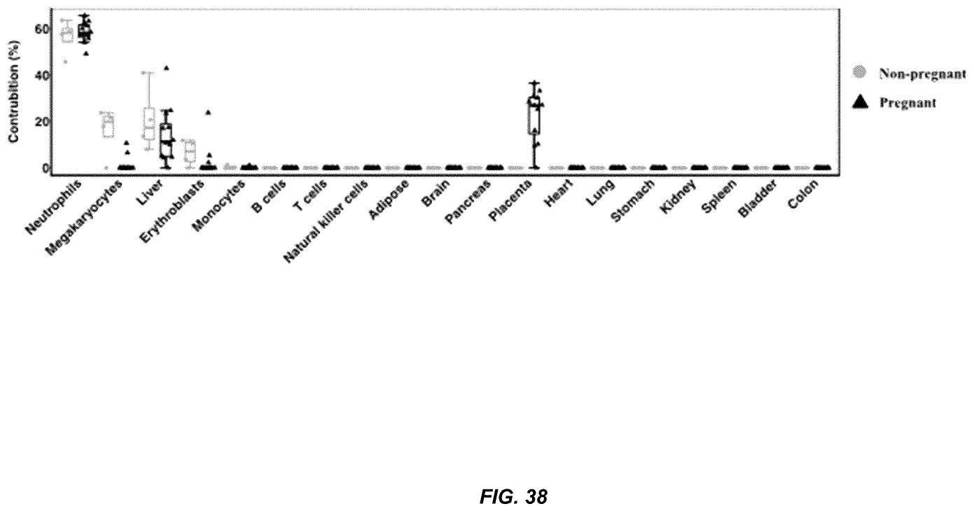

FIG. 38 shows a graph of the contribution percentage of different tissues for both pregnant and non-pregnant samples based on H3K4me3 histone modifications of cfDNA. The x-axis shows the tissue types. The y-axis shows the contribution percentage deduced by H3K4me3. The tissue types may include, but are not limited to, neutrophils, monocytes, B cells, T cells, natural killer cells, erythroblast cells, megakaryocytes, the liver, brain, pancreas, placenta, heart, colon, lung, adipose, kidney, spleen, bladder, and stomach. One could observe that the major contributors related to H3K4me3 in plasma DNA were blood-related cell types (i.e., megakaryocytes, neutrophils, and erythroblasts) for both pregnant and non-pregnant subjects, with a median contribution of 61.74% and 82.13%, respectively. Of note, the placental contribution of H3K4me3 was significantly higher in pregnant subjects (median: 27%; range: 0%-36.67%), compared with nonpregnant subjects with nearly no contributions (P value: 0.0081, Mann-Whitney U test). These results suggested that it was feasible to use histone modifications to deduce proportional contributions from various tissues into plasma DNA.

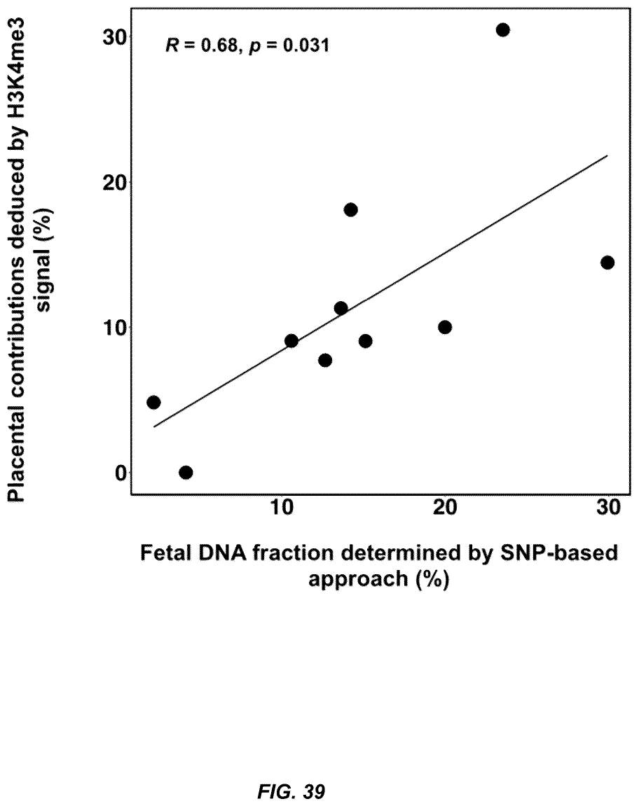

FIG. 39 shows a graph of the placental contributions determined by histone modification against the fetal DNA fraction. The placental contribution as a percentage deduced by H3K4me3 signal is on the y-axis. The fetal DNA fraction determined by a SNP-based approach is on the x-axis. The placental contributions deduced by ChIP signals of histone modifications according to the embodiments in this disclosure are well correlated with fetal DNA fraction deduced by SNP-based approach (Pearson's r: 0.68; P value: 0.031). These results suggested that the use of histone modifications enabled the determination of proportional contributions from various tissues into plasma DNA.

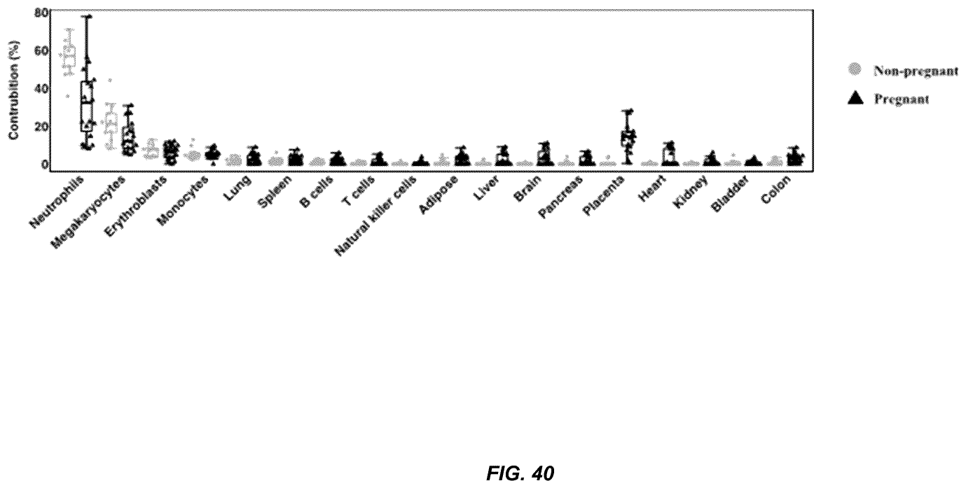

FIG. 40 is a graph of the contribution percentage of different tissues for both pregnant and non-pregnant samples based on H3K27ac histone modifications of cfDNA. The x-axis shows the tissue. The y-axis shows the tissue contribution deduced by H3K27ac histone modifications. FIG. 40 shows that the use of another type of histone modification such as H3K27ac can allow for deducing proportional DNA contributions of histone modifications from various tissues into plasma DNA. One could observe that the major contributors related to H3K27ac in plasma were blood-related cell types (i.e., megakaryocytes, neutrophils, and erythroblasts) for both pregnant and non-pregnant subjects with median contribution of 89.51% and 58.67%, respectively. Of note, the placental contribution of H3K27ac was significantly higher in pregnant subjects (median: 14.45%; range: 0.42-28.19%), compared with non-pregnant subjects (median: 0%; range: 0-4.01%) (P value: <0.0001, Mann-Whitney U test).

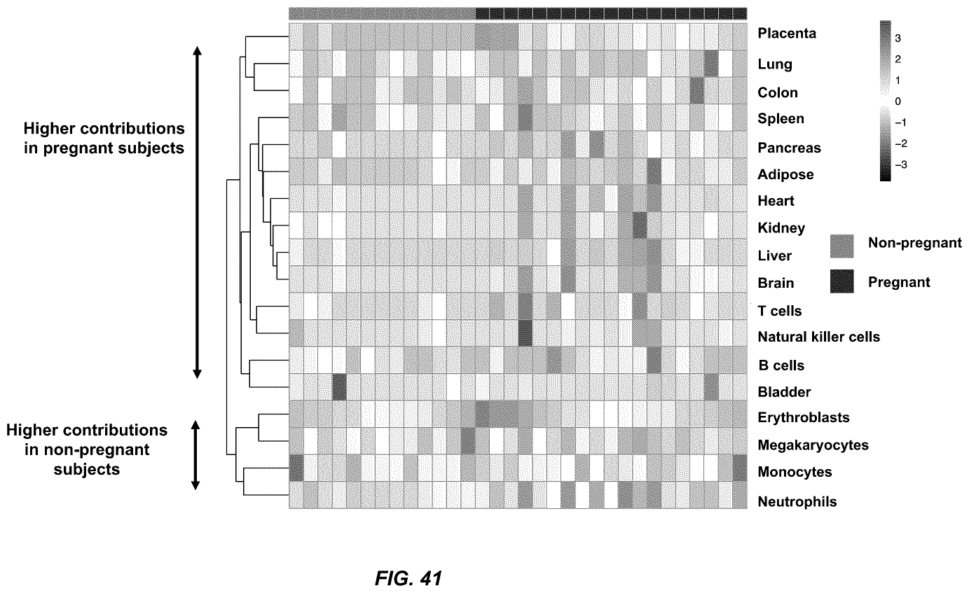

FIG. 41 shows a heatmap of tissue contributions deduced from H3K27ac ChIP signal in pregnant and non-pregnant subjects. The placental contribution of H3K27ac was significantly higher in pregnant subjects. However, tissues that are not typically associated with pregnant subjects have higher contributions of H3K27ac ChIP signal in pregnant subjects than non-pregnant subjects. Heatmap and clustering analysis of tissue contributions revealed tissue clusters (e.g., placenta, lung, colon, spleen, pancreas, adipose, heart, kidney) presenting higher contributions in pregnant subjects when compared with non-pregnant subjects. FIG. 41 shows that tissues common to both pregnant and non-pregnant subjects can have different contributions from a histone modification. The other tissue cluster composed of blood type cells (e.g., erythroblasts, megakaryocytes, neutrophils) presented relatively lower contribution in pregnant subjects.

2. Simultaneous Tissue Contribution Analysis

The particular tissue contribution of interest can be determined based on the deduced H3K27ac histone modification signals (ChIP signals in this disclosure) related to the tissue-specific histone modification regions. In one embodiment, the amount of histone modification may be deduced by fragmentomic features. In one embodiment, one can use various tissue-specific histone modification regions to analyze contributions from multiple tissue types simultaneously. As an example, we analyzed the plasma DNA samples of 8 healthy subjects. For each sample, we deduced the H3K27ac ChIP signal for the regions carrying histone modifications of H3K27ac specific to different tissues. The H3K27ac ChIP signals were deduced using the cumulative frequency of molecules within a size range of 230 to 350 bp.

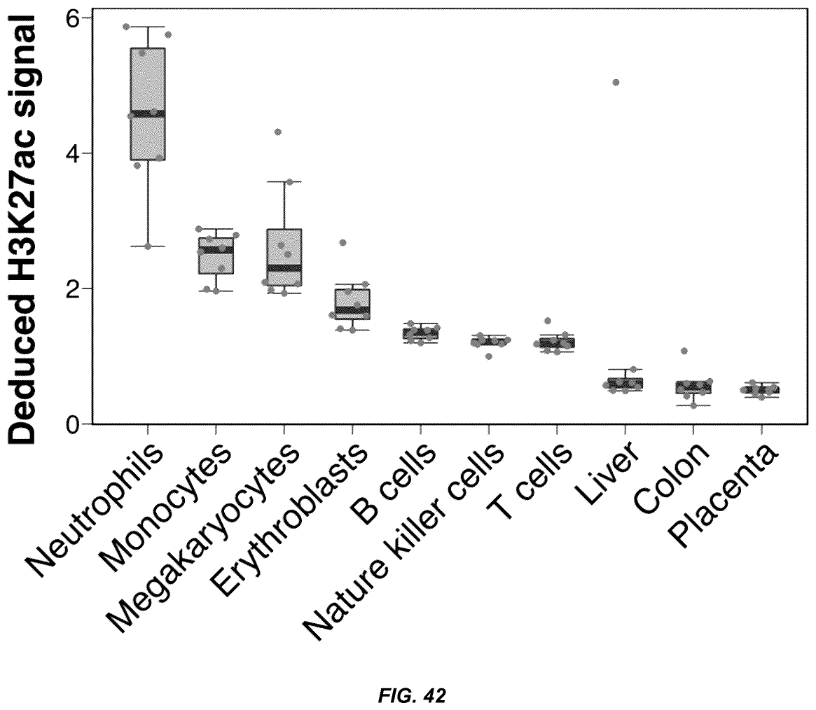

FIG. 42 shows a graph of the deduced H3K27ac signal versus the particular tissue. The deduced H3K27ac histone modification signals (also referred to as ChIP signals) is shown on the y-axis. The tissue-specific region is shown on the x-axis. Each dot represents one plasma DNA sample.

Comparing the deduced H3K27ac ChIP signals across various tissue specific regions, neutrophil-specific regions showed the highest median levels compared to other tissues, suggesting neutrophils as the major contributor for plasma cfDNA. The contribution of each tissue may related to the ChIP signal. For example, one may determine that monocytes and megakaryocytes may be the next major contributors. The tissues with the least contribution may be placenta and colon. These observations were in line with the previous studies for healthy individuals, by which neutrophils were proved to be the major contributor of the plasma DNA (K. Sun, et al., Proc Natl Acad Sci USA. 2015; 112; E5503-E5512).

3. Classifying Pregnant Subjects

ChIP signals may be used to determine the fetal DNA fraction or for differentiating pregnant and non-pregnant subjects.

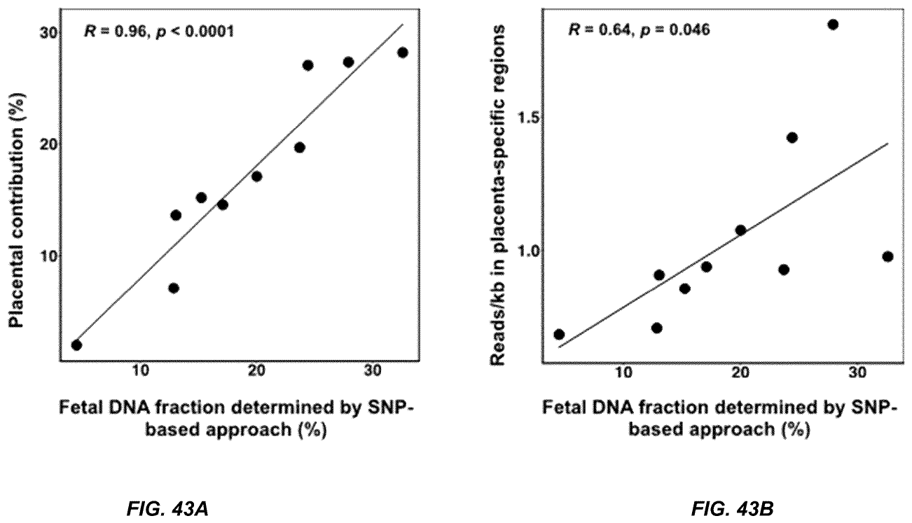

FIG. 43A and FIG. 43B are graphs showing the correlation between H3K27ac ChIP signals and the fetal DNA fraction determined by SNP-based approaches. The x-axis shows the fetal DNA fraction as a percentage as determined by an SNP-based approach. As seen in FIG. 43A, the use of H3K27ac signals allowed a higher correlation between placental contribution deduced by histone modifications and fetal DNA fraction deduced by SNP-based approach (Pearson's r: 0.96; P value: <0.0001). This result highlighted that, in some embodiments, the selective use of different types of histone modifications would improve the performance of plasma DNA deconvolution for tissue DNA contributions related to histone modifications. As seen in FIG. 43B, there was a weaker correlation between fetal DNA fraction and H3K27ac signal as reads/kb (in 1 million scale) in placenta-specific H3K27ac regions. (Pearson's r: 0.64; P value: <0.046).

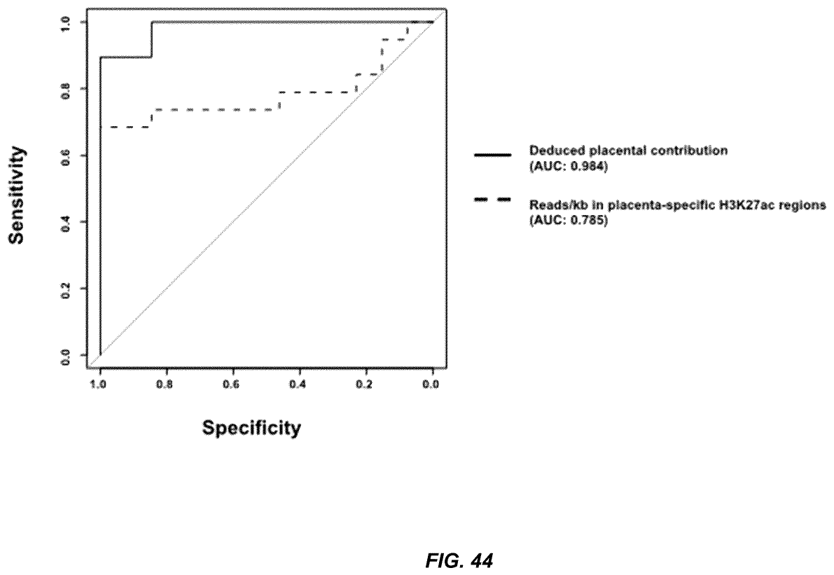

FIG. 44 is an ROC curve for differentiating pregnant and non-pregnant subjects. The x-axis shows specificity. The y-axis shows the sensitivity. The solid line shows using the deduced placental contribution from H3K27ac ChIP signals. The dashed line shows using the reads (in millions)/kb in placenta-specific H3K27ac regions. The deduced placental contribution technique has an AUC of 0.984 for differentiating between pregnant and non-pregnant subjects. The reads/kb technique (i.e., metric reported in Sadeh et al.'s study) has an AUC of 0.785. The results suggested that the use of deduced tissue contribution using quadratic programming gave a better classification performance, compared with the use of reads/kb.

4. Samples from Subjects with Cancer

In one embodiment, although there was no colon specific H3K4me3 regions (FIG. 37), one could still deduce the colon contribution using other tissue-specific and tissue-variable regions. We analyzed the raw sequencing data from Sadeh et al.'s study according to the embodiments of this disclosure.

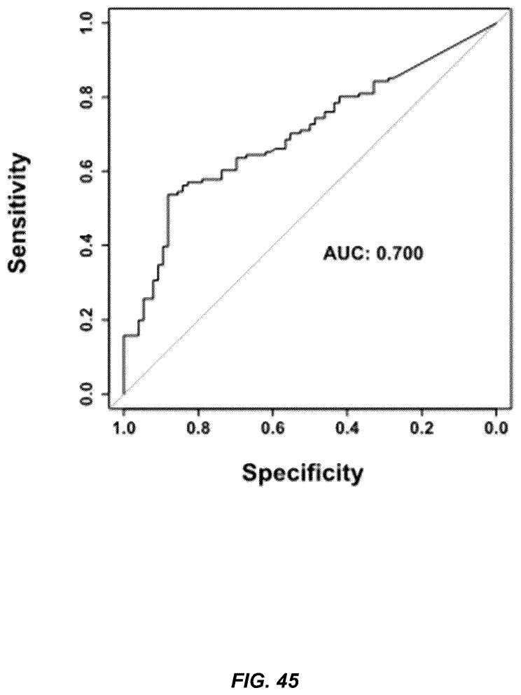

FIG. 45 shows a receiver operating characteristic (ROC) curve for differentiating control subjects and subjects with colorectal cancer (CRC) using deduced colon contributions. The ROC curve shows an area under the curve (AUC) of 0.7. The colon contribution may serve as an indicator for differentiating subjects with CRC from control subjects. In some embodiments, one could use only tissue-variable regions.

C. Detecting and Monitoring Diseases

Histone modification levels measured by embodiments in this disclosure can be used to determine a classification of a likelihood of a blood disorder and a classification of a level of cancer, including whether the cancer has metastasized. Biological samples from subjects with beta-thalassemia major were analyzed for histone modification levels. Beta-thalassemia major is an example of a blood disorder. Other blood disorders would be expected to have similar anomalous results at least because blood disorders may have abnormal contributions from cells in the blood. Biological samples from subjects with colorectal cancer (CRC), were analyzed for histone modification levels. CRC is an example of a cancer. Other cancers would be expected to have similar histone modification levels when the cancer is localized to a tissue or when the cancer metastasized to another tissue.

1. Blood Disorders

To demonstrate the clinical utility with the use of histone modification-based plasma DNA tissue deconvolution, we recruited patients with hematological diseases such as, but not limited to, beta-thalassemia major, iron deficiency anemia, aplastic anemia, and idiopathic thrombocytopenia purpura. We applied H3K27ac based immunoprecipitation assay followed by massively parallel sequencing to those plasma DNA samples.

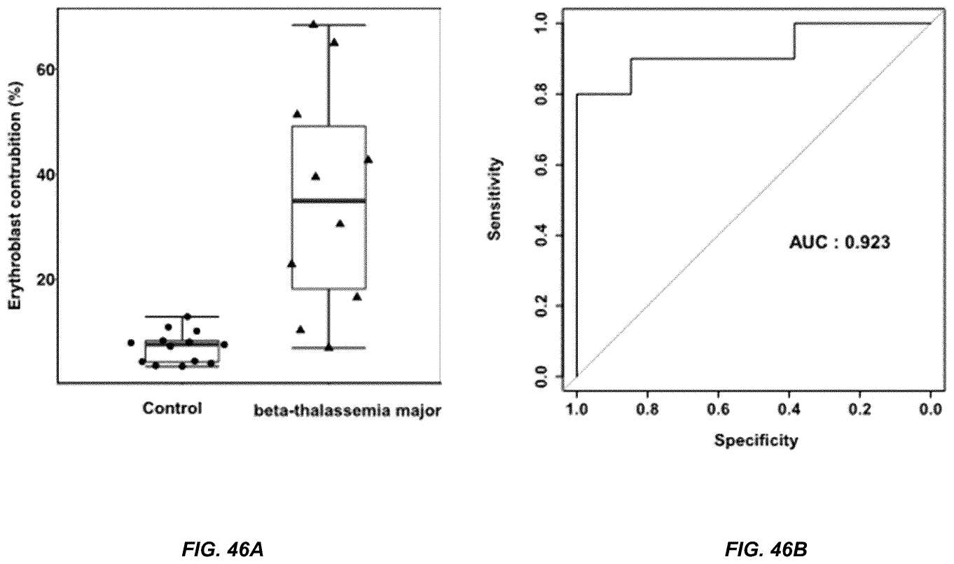

FIG. 46A is a graph comparing erythroblast contributions deduced by H3K27ac ChIP signals between subjects with beta-thalassemia major and control subjects without beta-thalassemia major. The x-axis shows the subject category. The y-axis shows the erythroblast contribution in percent deduced by H3K27ac ChIP signals. In FIG. 46A, compared with healthy control subjects (median: 7.54%; range: 0-12.85%), those subjects with beta-thalassemia major exhibited an aberrant contribution from erythroblasts (median: 34.97%; range: 6.89-68.44%) (P value: 0.00024, Mann-Whitney U test).

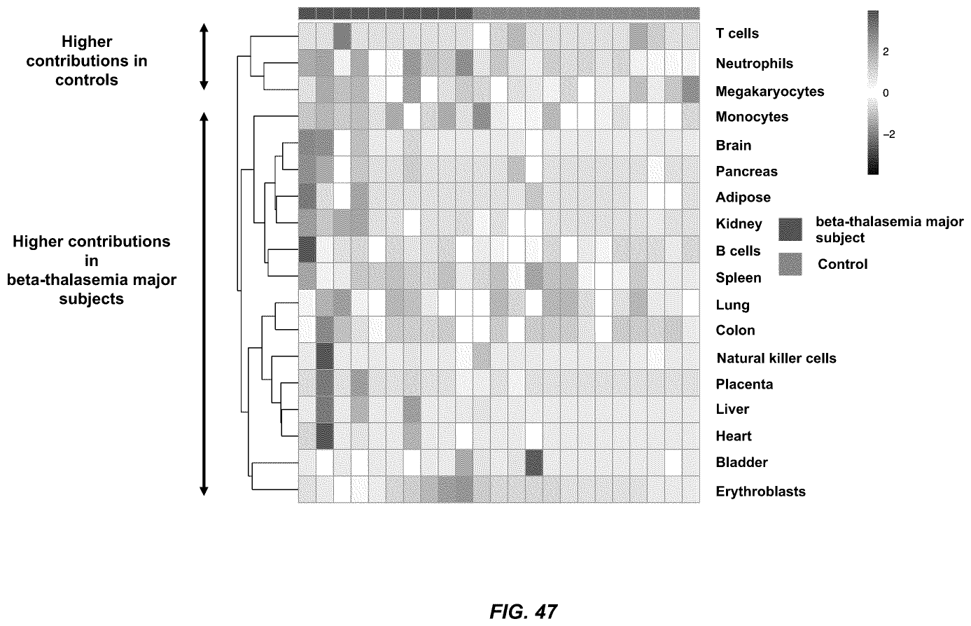

FIG. 46B is an ROC curve for using the deduced erythroblast contribution to differentiate subjects with and without beta-thalassemia major. The x-axis is the specificity. The y-axis is sensitivity. A ROC analysis revealed that one could achieve AUC of 0.923 by deduced erythroblast contribution in erythroblast-specific regions, suggesting that the use of histone modification based plasma DNA tissue deconvolution would enable the detection and/or monitoring of hematological disorders (e.g., beta-thalassemia major). The deduced tissue contribution was superior to the regional signal measured by reads/kb, which had an AUC of FIG. 47 is a heatmap of tissue contributions deduced using H3K27ac ChIP signals in subjects with beta-thalassemia major and control subjects. Tissues clustered and separated by tissue contribution under different pathological conditions. Erythroblasts, monocytes, brain, and others presented higher contribution in beta-thalassemia major subjects when compared with control subjects. T cells, neutrophils, and megakaryocytes presented lower contribution in beta-thalassemia major subjects. In addition, we observed a lower erythroblast contribution (1.62%) in a subject with aplastic anemia and a higher erythroblast contribution (16.07%) in a subject with iron deficiency anemia compared with the median level of erythroblast contributions in control subjects (7.54%). These results were consistent with the previous findings which also observe the similar trends by droplet digital PCR (ddPCR) assay using methylation markers (Lam, et al. Clin Chem. 2017; 63:1614-1623). These results suggest the possible clinical utilities by histone modification-based plasma DNA tissue deconvolution.

In addition, we used a published ddPCR assay to measure erythroid DNA in those plasma DNA samples using a differentially methylated region that was hypomethylated in erythroblasts but hypermethylated in other cell types (Lam et al. Clin Chem. 2017; 63:1614-1623).

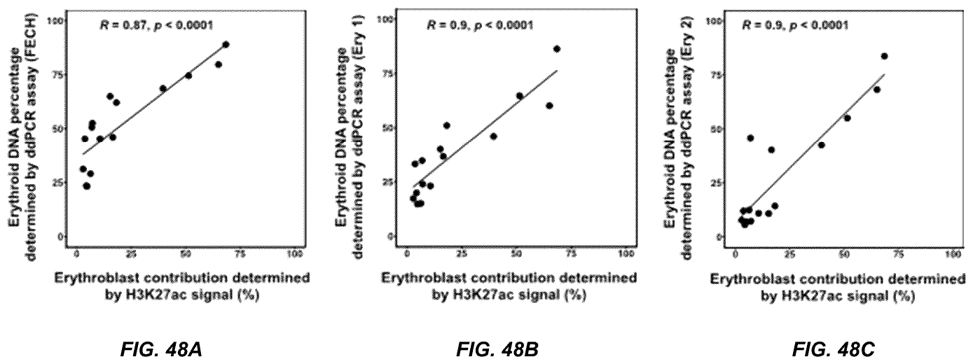

FIGS. 48A, 48B, and 48C show correlation between erythroid DNA percentage determined by ddPCR assay and the erythroblast contribution determined by H3K27ac signal. The x-axis shows the erythroblast contribution determined by H3K27ac signal. The y-axis shows the erythroid DNA percentage determined by ddPCR assay. FIG. 48A shows use of the FECH (chr18:55250563-55250585) marker, which has a Pearson's r of 0.87 and a P value<0.0001. FIG. 48B shows use of the Ery 1 (chr12:48227688-48227701) marker, which has a Pearson's r of 0.90 and a P value<0.0001. FIG. 48C shows use of the Ery 2 (chr12:48228144-48228167) marker, which has a Pearson's r of 0.90 and a P value<0.0001. The data in these figures further suggested that the use of histone modifications enabled an accurate deduction of proportional contributions from various tissues into plasma DNA.

2. Cancer with Metastasis

The deduced ChIP signal of histone modification from plasma DNA without immunoprecipitation can be used to differentiate between localized cancer and metastatic cancer. We analyzed a cohort of 4 localized colorectal cancer (CRC) patients, 7 CRC patients with liver metastasis, and 8 healthy control samples. For each sample, we deduced the H3K27ac ChIP signals for colon- and liver-specific regions. The H3K27ac ChIP signals were deduced using the cumulative frequency of molecules within a size range of 230 to 350 bp.

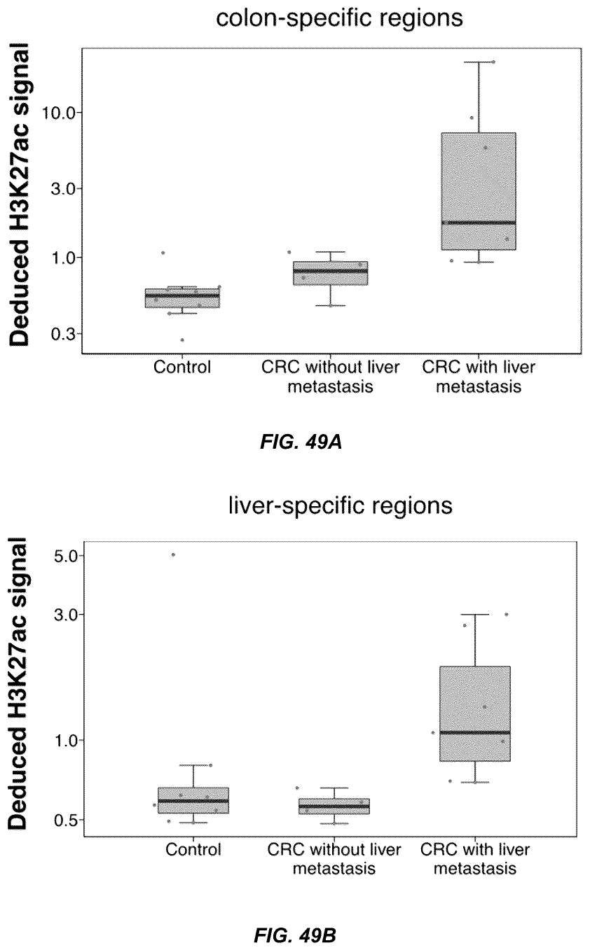

FIG. 49A is a graph comparing plasma DNA results from healthy controls to subjects with CRC in colon-specific H3K27ac regions. The graph shows the deduced H3K27ac signal on the y-axis and the type of subject (healthy, CRC without liver metastasis, and CRC with liver metastasis) on the x-axis. Each dot represents one plasma DNA sample. The deduced H3K27ac signal in heathy subjects (median: 0.54; range: 0.27-1.08) was lower than the levels in localized (i.e., without liver metastasis) CRC patients (median: 0.81; range: 0.47-1.09) and CRC patients with liver metastasis (median: 1.73; range: 0.93-22.28).

FIG. 49B is a graph comparing plasma DNA results from healthy controls to subjects with CRC in liver-specific H3K27A regions. The graph shows the deduced H3K27ac signal on the y-axis and the type of subject (healthy, CRC without liver metastasis, and CRC with liver metastasis) on the x-axis. Each dot represents one plasma DNA sample. The deduced H3K27ac levels for the liver-specific H3K27ac regions were shown to be exclusively increased in CRC patients with liver metastasis, indicating the increase of liver contribution to cfDNA caused by the liver metastasis. Taking data from both colon-specific regions and liver-specific regions, deduced ChIP signals can be used to differentiate between localized and metastatic cancer patients, which may be informative for clinical management.

D. Example of Urine DNA Tissue Mapping

We have illustrated that the relative tissue contribution to the plasma DNA pool can be deduced by comparing the profile of plasma DNA histone modifications with profiles of histone modifications derived from a number of organs, tissues, or cells. We further demonstrated that these methods present in this disclosure could be extended to urine samples.

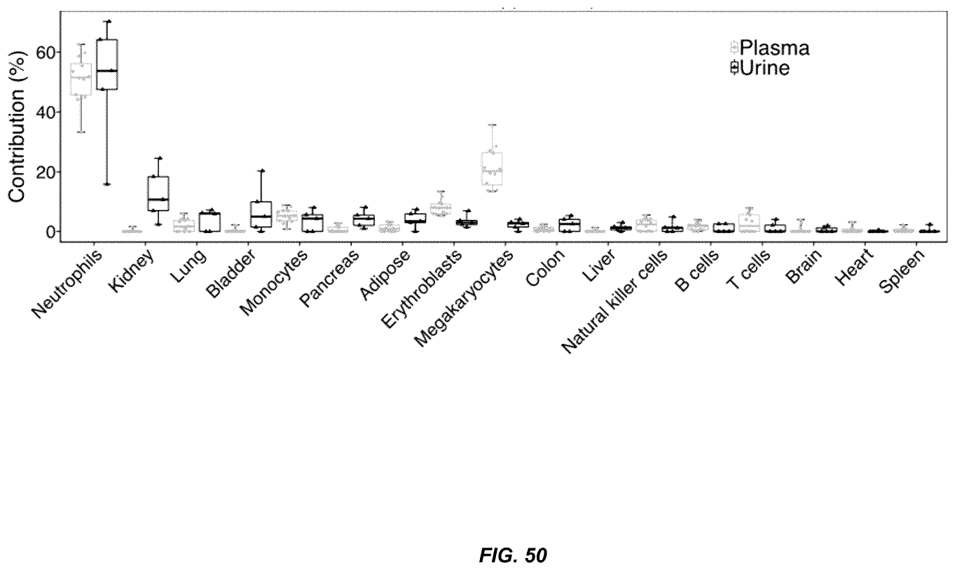

FIG. 50 is a graph of the tissue contributions in urine and plasma samples. The x-axis shows the type of tissue. The y-axis shows the percent contribution of the tissue. Each tissue includes two boxplots. The first boxplot (in gray) represents data from plasma samples. The second boxplot (in black) represents data from urine samples. For urine samples, the contribution was deduced by comparing the profile of urinary DNA histone modifications (e.g., H3K27ac) with profiles of histone modifications derived from reference organs, tissues, or cells. For plasma samples, the contribution was similarly deduced by comparing the profile of plasma DNA histone modifications with profiles of histone modifications derived from reference organs, tissues, or cells.

The urine DNA samples showed significantly higher percentage contributions of kidney (median: 10.66%) and bladder (median: 4.98%) than counterparts in plasma DNA samples (median of kidney: 0.00%, median of bladder: 0.00%), which is expected from urine samples. These results demonstrate that urine samples can be used to determine tissue contribution using deduced histone modification levels.

E. Example Method for Determining Fractional Concentration

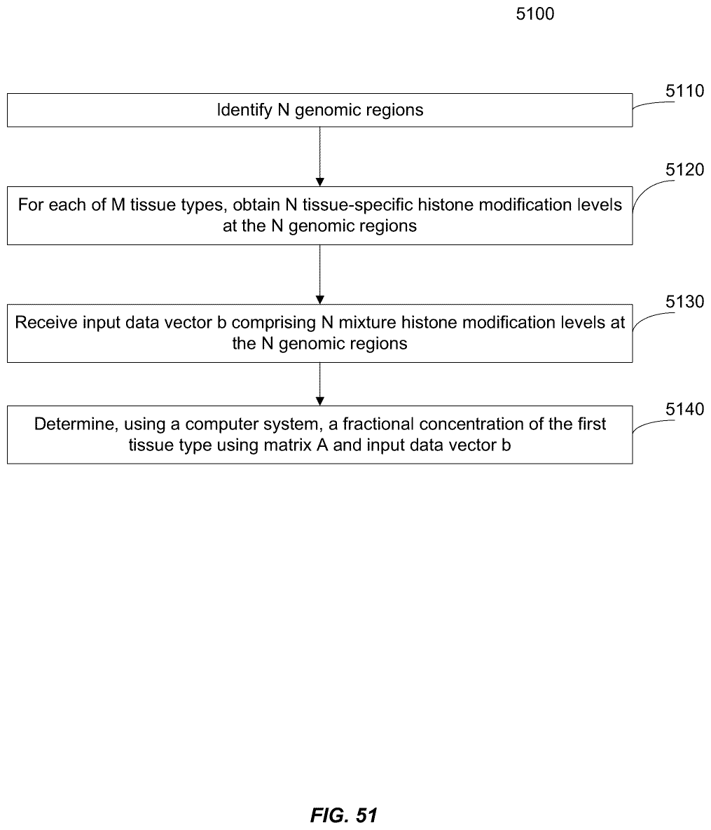

FIG. 51 is a flowchart of an example process 5100 associated with determining a fractional concentration of a tissue type. In some implementations, one or more process blocks of FIG. 51 may be performed by a system (e.g., measurement system 5900). In some implementations, one or more process blocks of FIG. 51 may be performed by another device or a group of devices separate from or including the system. Additionally, or alternatively, one or more process blocks of FIG. 51 may be performed by one or more components of measurement system 5900, such as assay 5908, assay device 5910, detector 5920, logic system 5930, local memory 5935, external memory 5940, storage device 5945, and/or processor 5950.

At block 5110, N genomic regions are identified. N is an integer greater than 1. The N genomic regions may be regions that are known to carry tissue-specific histone modifications. The region may be determined by criteria described herein. For instance, the region may have a histone modification level for a tissue that is greater than a cutoff amount. The cutoff amount may be a normalized ChIP signal, be based on a relative percentage difference, and/or based on a coefficient of variation across all tissue types. The region may be any region of interest described herein.

At block 5120, for each of M tissue types, N tissue-specific histone modifications levels at the N genomic regions are obtained. N is greater than or equal to M. The histone modification may be H3K27ac, H3K4me3, or any histone modification described herein. The tissue histone modification levels form a matrix A of dimensions N by M. One of the M tissue types corresponds to a first tissue type. The first tissue type may be fetal, erythroblast, any tissue listed in FIG. 37 or FIG. 38, or any tissue described herein. At least one genomic region of the N genomic regions includes non-zero histone modification levels from at least two of the M tissue types. For example, at least one histone modification level may not be exclusive to a single tissue.

At block 5130, an input data vector b is received. The input data vector b may include N mixture histone modification levels at the N genomic regions. The N mixture histone modification levels may be measured from a plurality of cell-free DNA molecules in a biological sample of a subject. The biological sample may be any biological sample described herein. The N mixture histone modification levels may be measured by cell-free chromatin immunoprecipitation followed by sequencing (cfChIP-seq), by determining one or more relative frequencies of a set of one or more sequence motifs in the plurality of cell-free DNA molecules, or by determining one or more relative frequencies of one or more size ranges in the plurality of cell-free DNA molecules. Relative frequencies of fragmentomic features other than sequence motifs and size ranges can also be used. The mixture histone modification levels may be determined by any method described herein.

At block 5140, a fractional concentration of the first tissue type is determined, using a computer system and using matrix A and input data vector b. The fractional contribution may be determined using quadratic programming.

Process 5100 may include determining classifications using the fractional concentration. For example, the first tissue type may be a fetal tissue, and process 5100 may further include determining a classification of a pregnancy in the subject using the fractional concentration of the first tissue type. The classification of the pregnancy may be whether the pregnancy exists, a gestational age (e.g., trimester) of the fetus, or a level (e.g., existence) of a pregnancy-associated disorder.

As another example, process 5100 may include determining a classification of a disease using the fractional concentration of the first tissue type. For example, the disease may be beta-thalassemia major, iron deficiency anemia, aplastic anemia, or idiopathic thrombocytopenia purpura. The first tissue type may be erythroblasts, monocytes, brain, T cells, neutrophils, megakaryocytes, or any other tissue described herein. The level of the disease may be whether the disease exists or a severity of the disease. The disease may be a disease (e.g., cancer) of the first tissue type.

Although FIG. 51 shows example blocks of process 5100, in some implementations, process 5100 may include additional blocks, fewer blocks, different blocks, or differently arranged blocks than those depicted in FIG. 51. Additionally, or alternatively, two or more of the blocks of process 5100 may be performed in parallel.

F. Example Method for Determining Classification of Pregnancy or Disease

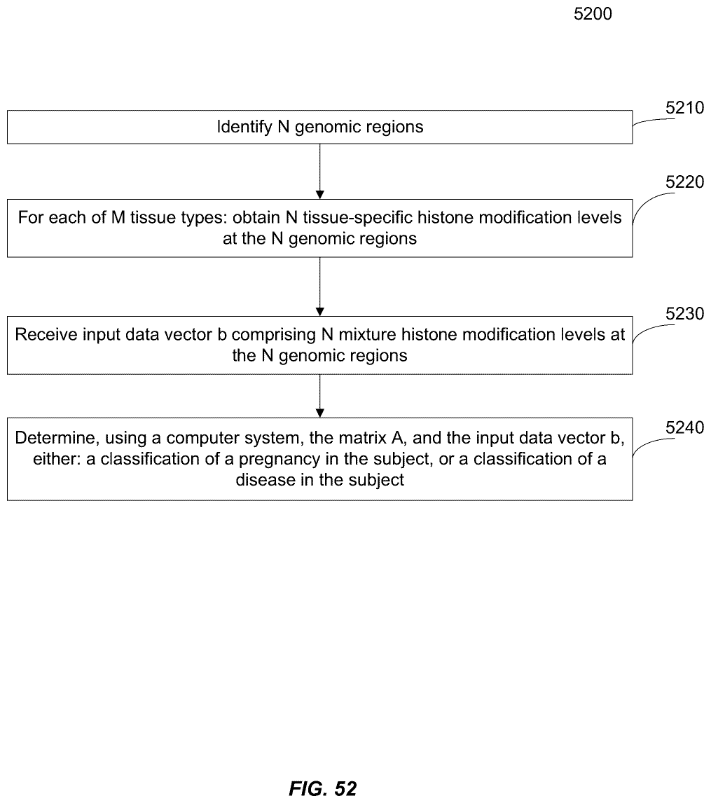

FIG. 52 is a flowchart of an example process 5200 associated with determining a fractional concentration of a tissue type. In some implementations, one or more process blocks of FIG. 52 may be performed by a system (e.g., measurement system 5900). In some implementations, one or more process blocks of FIG. 52 may be performed by another device or a group of devices separate from or including the system. Additionally, or alternatively, one or more process blocks of FIG. 52 may be performed by one or more components of measurement system 5900, such as assay 5908, assay device 5910, detector 5920, logic system 5930, local memory 5935, external memory 5940, storage device 5945, and/or processor 5950.

At block 5210, N genomic regions are identified. Block 5210 may be performed in the same manner as block 5110.

At block 5220, for each of M tissue types, N tissue-specific histone modifications levels at the N genomic regions are obtained. N is greater than or equal to M. Block 5220 may be performed in the same manner as block 5120.

At block 5230, an input data vector b is received. Block 5230 may be performed in the same manner as block 5130.

At block 5240, either a classification of a pregnancy in the subject or a classification of a disease in the subject may be determined using a computer system, the matrix A, and the input data vector b. The classification of the pregnancy or the classification of the disease may be any classification described with process 5100. Process 5200 may determine the classification without determining a fractional concentration of a tissue type.

Determining the classification of the pregnancy or the classification of the disease may include inputting the matrix A and the input data vector b into a model (e.g., a machine learning model). The model may be trained by receiving the matrix A and a plurality of training input data vectors b obtained from a plurality of biological samples of a plurality of training subjects. Each training subject may have a known classification of a condition of the training subject. The condition may be a status of a pregnancy or a known classification of the disease or any condition described herein. A plurality of training samples may be stored. Each training sample may include one of the plurality of training input data vectors b and a first label indicating the known classification of the condition. Parameters of the model may be optimized, using the plurality of training samples, based on outputs of the model matching or not matching corresponding labels of the first labels when the matrix A and the plurality of training input data vectors b are input to the model. An output of the model may specify the classification of the condition. The classification of the condition may be determined using the model.

The model may include a convolutional neural network (CNN). The CNN may include a set of convolutional filters configured to filter the plurality of input data vectors b. The filter may be any filter described herein. The number of filters for each layer may be from 10 to 20, 20 to 30, 30 to 40, 40 to 50, 50 to 60, 60 to 70, 70 to 80, 80 to 90, 90 to 100, 100 to 150, 150 to 200, or more. The kernel size for the filters can be 2, 3, 4, 5, 6, 7, 8, 9, 10, 11, 12, 13, 14, 15, from 15 to 20, from 20 to 30, from 30 to 40, or more. The CNN may include an input layer configured to receive the filtered plurality of input data vectors b. The CNN may also include a plurality of hidden layers including a plurality of nodes. The first layer of the plurality of hidden layers coupled to the input layer. The CNN may further include an output layer coupled to a last layer of the plurality of hidden layers and configured to output an output data structure. The output data structure may include the properties.

The model may include a supervised learning model. Supervised learning models may include different approaches and algorithms including analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, Nearest Neighbor Algorithm, probably approximately correct learning (PAC) learning, ripple down rules, a knowledge acquisition methodology, symbolic machine learning algorithms, subsymbolic machine learning algorithms, support vector machines, Minimum Complexity Machines (MCM), random forests, ensembles of classifiers, ordinal classification, data pre-processing, handling imbalanced datasets, statistical relational learning, or Proaftn, a multicriteria classification algorithm The model may linear regression, logistic regression, deep recurrent neural network (e.g., long short term memory, LSTM), Bayes classifier, hidden Markov model (HMM), linear discriminant analysis (LDA), k-means clustering, density-based spatial clustering of applications with noise (DB SCAN), random forest algorithm, support vector machine (SVM), or any model described herein.

As part of training a machine learning model, the parameters of the machine learning model (such as weights, thresholds, e.g., as may be used for activation functions in neural networks, etc.) can be optimized based on the training samples (training set) to provide an optimized accuracy in classifying the modification of the nucleotide at the target position. Various form of optimization may be performed, e.g., backpropagation, empirical risk minimization, and structural risk minimization. A validation set of samples (data structure and label) can be used to validate the accuracy of the model. Cross-validation may be performed using various portions of the training set for training and validation. The model can comprise a plurality of submodels, thereby providing an ensemble model. The submodels may be weaker models that once combined provide a more accurate final model.

Although FIG. 52 shows example blocks of process 5200, in some implementations, process 5200 may include additional blocks, fewer blocks, different blocks, or differently arranged blocks than those depicted in FIG. 52. Additionally, or alternatively, two or more of the blocks of process 5200 may be performed in parallel.

### VI. Disorder Detection Using Sequence Motifs and Fragment Sizes

In embodiments, one or both of fragment sizes and sequence motifs can be used to classify a pregnancy or disorder. For example, end motifs can be used as described elsewhere in this application, including in section III.D and process 2300 of FIG. 23. Sizes of fragments, which may not be limited to certain end motifs, may be used. A machine learning model may use the end motifs and/or fragment sizes to classify a pregnancy or disorder.

A. Example Results

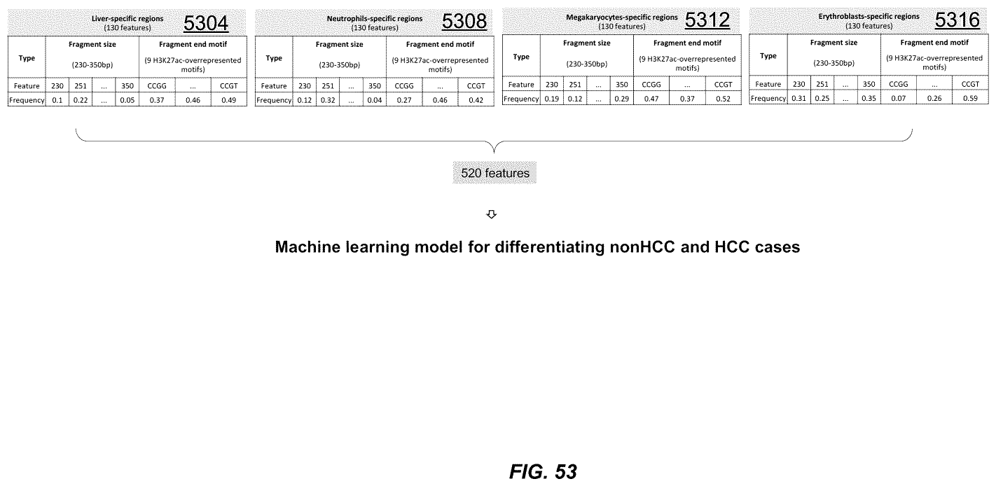

FIG. 53 illustrates input features included in a machine learning model to differentiate between hepatocellular carcinoma (HCC) and non-HCC cases. Arrays 5304, 5308, 5312, and 5316 each include data from a tissue-specific region. The tissue-specific regions include liver-specific regions, neutrophils-specific regions, megakaryocytes-specific regions, and erythroblasts-specific regions. Each array includes fragment size and fragment end motif information. The frequencies of all molecules within 230-350 nt (the molecules not being limited to any specific fragment end motifs when considering size) are in each array. For example, in array 5304, fragments aligning to liver-specific regions having a size of 230 have a frequency of relative to other sizes in the liver-specific regions. Other size ranges are also possible.

The arrays also include frequencies of all molecules with the 9 H3K27ac-associated end motifs (the molecules not being limited to any fragment size when considering end motifs. H3K27ac-associated end motifs include, but are not limited to CCGG, CCGC, GCGG, TCGG, TCGC, CCGA, CCCG, GCGC, and/or CCGT. The H3K27ac-associated end motifs may be defined by end motifs that are overrepresented in regions with high H3K27ac signal compared to regions with low H3K27ac signal in the sequenced result of plasma DNA samples without immunoprecipitation. For example, the overrepresentation may be a fold change in an end motif frequency of 1×, 2×, 3×, 4×, 5×, 6×, 7×, 8×, 9×, 10×, 20×, 30×, 50×, etc, when comparing the results of plasma DNA samples in regions with high and low H3K27ac signal. In some embodiments, the H3K27ac-associated end motifs may be defined by those motifs that are overrepresented in the sequenced result of plasma DNA samples with immunoprecipitation compared to the result of plasma DNA samples without immunoprecipitation. For example, the overrepresentation may be a fold change in an end motif frequency of 1×, 2×, 3×, 4×, 5×, 6×, 7×, 8×, 9×, 10×, 20×, 30×, 50×, etc, when comparing the results of plasma DNA samples with and without immunoprecipitation.

The data from all arrays (i.e., one larger array or a matrix) can be input into a machine learning model to differentiate between non-HCC and HCC subjects. A machine learning model may include, but is not limited to, support vector machine, random forest, convolutional neural network, or any model described herein. In this example, there are a total of 130 features for one type of tissue-specific H3K27ac-related region. With the four different tissue-specific regions, there are 520 features.

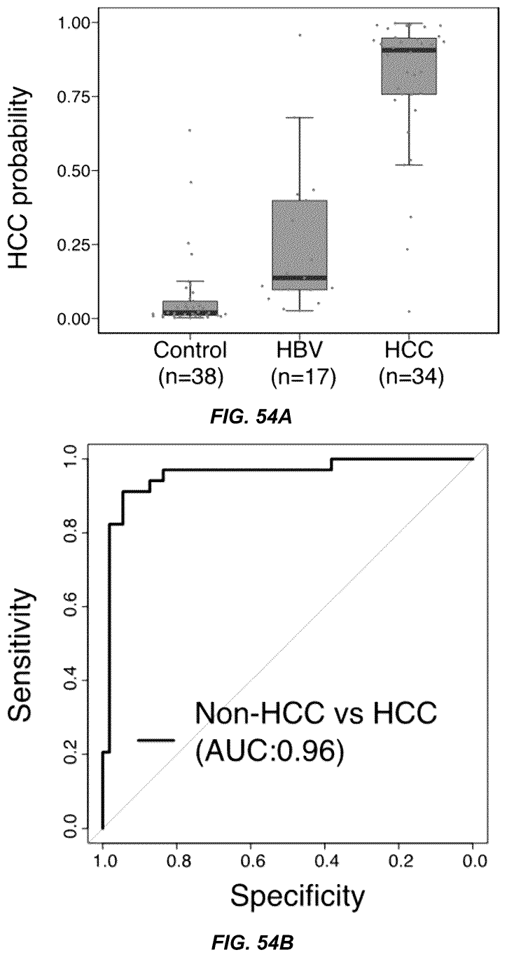

FIG. 54A and FIG. 54B show results from a machine learning model using the features illustrated in FIG. 53. FIG. 54A shows the probability of HCC determined by the machine learning model for control subjects, subjects with chronic hepatitis B virus (HBV), and subjects with HCC. The y-axis is the HCC probability. The x-axis shows the type of subject. FIG. 54A shows that the HCC probability determined by the aforementioned machine learning model was significantly higher in HCC patients compared with patients without HCC.

FIG. 54B is a receiver operating characteristic (ROC) curve. Sensitivity is on the y-axis. Sensitivity is on the x-axis. The ROC analysis revealed that one could achieve area under the curve (AUC) of 0.96 for differentiating non-HCC and HCC cases by the HCC probability.

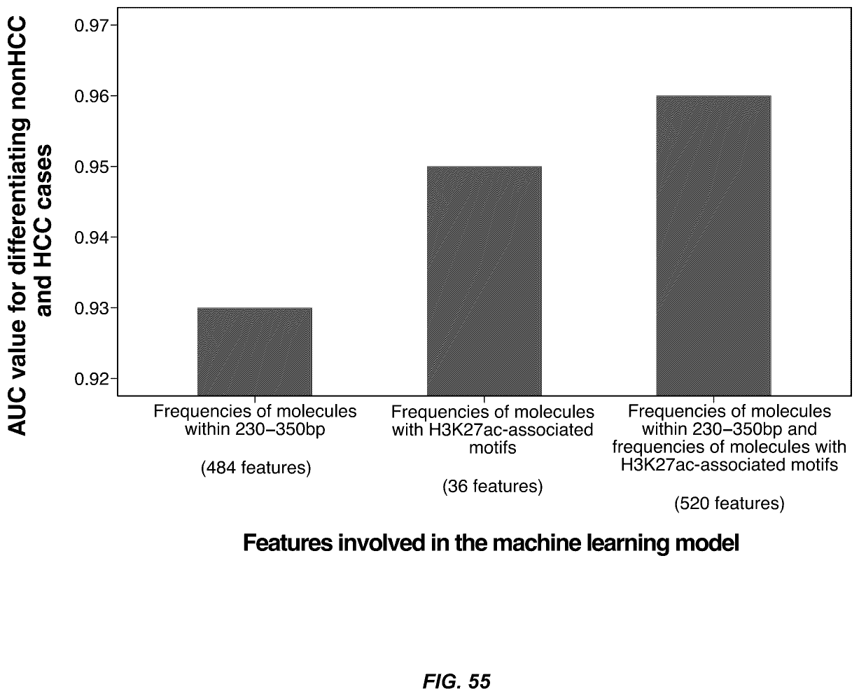

FIG. 55 is a figure showing AUC values determined using different fragmentomics features for differentiating non-HCC and HCC cases. The y-axis shows the AUC value. The x-axis shows the different fragmentomics features used in machine learning models to differentiate between non-HCC and HCC cases. The first column shows an AUC of 0.93 using the frequencies of molecule sizes within 230 to 350 bp. This model includes 484 features (121 different sizes and 4 tissue-specific regions). The second column shows an AUC of 0.95 using the frequencies of molecules with H3K27ac-associated motifs. This model uses 36 features (9 motifs and 4 tissue-specific regions). The third column has an AUC of 0.96 using both the frequencies of molecule sizes within 230 to 350 bp and the frequencies of H3K27ac-associated motifs. This model uses 520 features and is described with FIGS. 53 and 54. FIG. 55 shows that combining size frequencies and motif frequencies improve accuracy of determining HCC cases. FIG. 55 also shows that size frequencies and motif frequencies individually for different tissue-specific regions can be used to differentiate HCC cases from non-HCC cases.

B. Example Method

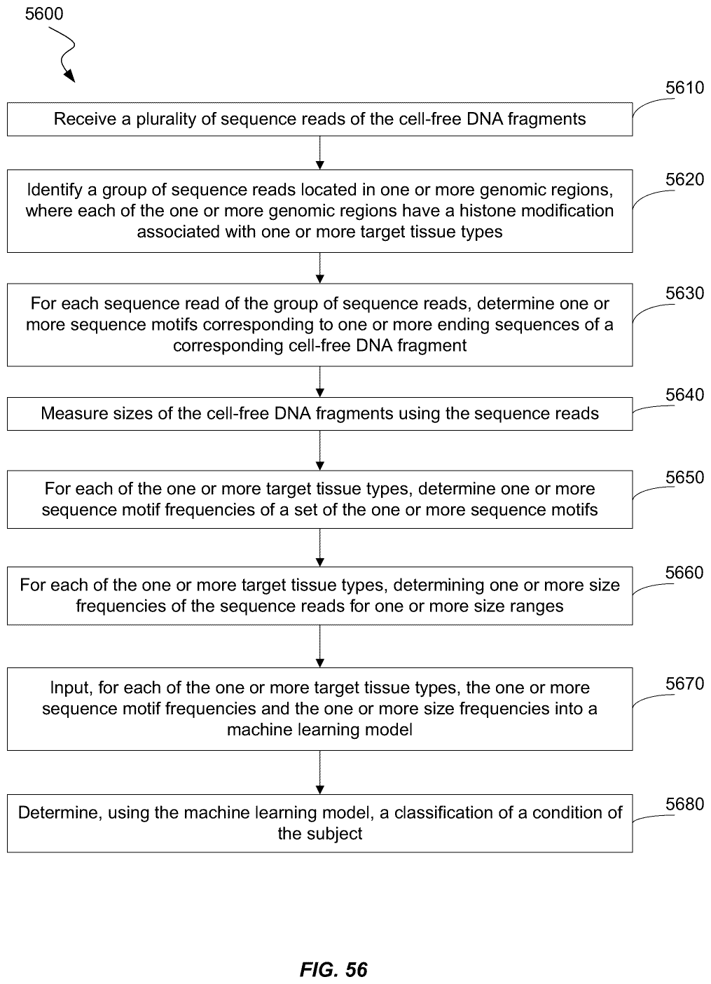

FIG. 56 is a flowchart of an example process 5600 of analyzing a biological sample of a subject to determine a classification of a condition of the subject. The biological sample includes cell-free DNA fragments. In some implementations, one or more process blocks of FIG. 56 may be performed by a system (e.g., measurement system 5900). In some implementations, one or more process blocks of FIG. 56 may be performed by another device or a group of devices separate from or including the system. Additionally, or alternatively, one or more process blocks of FIG. 56 may be performed by one or more components of measurement system 5900, such as assay 5908, assay device 5910, detector 5920, logic system 5930, local memory 5935, external memory 5940, storage device 5945, and/or processor 5950. Process 5600 may include aspects described with process 1700.

At block 5610, a plurality of sequence reads of the cell-free DNA fragments is received. The plurality of sequence reads may include ending sequences corresponding to ends of the cell-free DNA fragments.

At block 5620, a group of sequence reads located in one or more genomic regions is identified. Each of the one or more genomic regions may have a histone modification associated with one or more target tissue types. The one or more target tissue types may include an organ that has cancer or fetal tissue. In some embodiments, the one or more target tissue types may include liver, neutrophils, megakaryocytes, or erythroblasts. The histone modification may be H3K4me1, H3K4me2, H3K27me3, H3K27ac, H3K36me3, H3K9me2, H3K9me3, H3S10P, H3R2me, H3T2P, H3K14ac, H3K9ac, H3K79me2, H3K79me3, H4K5ac, H4K8ac, H4K12ac, H4K16ac, H4K20me, H2BK120ub, or H2AK119ub.

At block 5630, one or more sequence motifs corresponding to one or more ending sequences of a corresponding cell-free DNA fragment are determined for each sequence read of the group of sequence reads. The set of the one or more sequence motifs may include 1 to 5, 5 to 11 to 15, 15 to 20, or 20 to 25 sequence motifs. The cell-free DNA fragments may consist of fragments with a sequence motif of the set of the one or more sequence motifs.

At block 5640, sizes of the cell-free DNA fragments using the sequence reads are measured. The cell-free DNA fragments may have sizes with a predetermined size range. The predetermined size range may be any size range described herein, including 230-350 nt.

At block 5650, one or more sequence motif frequencies of a set of the one or more sequence motifs are determined for each of the one or more target tissue types. The set of the one or more sequence motifs occurs at a higher rate in chromatin immunoprecipitation followed by sequencing (ChIP-seq) for the histone modification associated with the one or more genomic regions than in sequencing without chromatin immunoprecipitation.

At block 5660, one or more size frequencies of the sequence reads for one or more size ranges are determined for each of the one or more target tissue types.

At block 5670, the one or more sequence motif frequencies and the one or more size frequencies for each of the one or more target tissue types are input into a machine learning model. The machine learning model may include support vector machine, random forest, or convolutional neural network. The machine learning model may be any machine learning model disclosed herein, including a similar model to one described with process 5200.

The machine learning model may be trained by receiving a training data set. The training data set may include for each of the one or more target tissue types, training sequence motif frequencies of the set of the one or more sequence motifs and training size frequencies of cell-free DNA fragments from a plurality of biological samples of a plurality of training subjects. Each training subject may have a known classification of a condition.

The machine learning model may also be trained by storing a plurality of training samples. Each training sample may include for each of the one or more target tissue types, one or more training sequence motif frequencies of the set of the one or more sequence motifs occurring in cell-free DNA fragments in the training sample. Each training sample may include for each of the one or more target tissue types, training size frequencies of the cell-free DNA fragments in the training sample. Each training sample may also include a first label indicating a known classification of a condition.

The machine learning model may be trained by optimizing, using the plurality of training samples, parameters of the machine learning model based on outputs of the machine learning model matching or not matching corresponding labels of the first labels when the sequence motif frequencies and the size frequencies for each of the one or more target tissue types are input to the machine learning model. An output of the machine learning model may specify the classification of the condition.

In some embodiments, process 5600 may include, for each sequence motif of the set of the one or more sequence motifs, determining a size parameter of fragments having the respective sequence motif. A size parameter may be a statistical value (e.g., mean, median, mode, percentile) of the fragments having the respective sequence motif. Process 5600 may further include inputting the one or more size parameters into the machine learning model. The machine learning model in these embodiments may be trained with training samples including the determined size parameters.

At block 5680, a classification of a condition of a subject is determined using the machine learning model. The condition may be a pregnancy. For example, the classification of the pregnancy may provide a gestational age or the existence or severity of a pregnancy-associated disorder, including any pregnancy-associated disorder described herein. The condition may be a disease. The classification of the disease may be the existence or severity of the disease. The disease may be cancer, including hepatocellular carcinoma (HCC) or any cancer described herein.

In some embodiments, process 5600 may be modified such that either the sequence motif frequencies or the size frequencies are used. For example, process 5600 may include using only the size frequencies of molecules within a certain size range (e.g., first column in FIG. 55). In this case, block 5630 and block 5650 are optional. Block 5670 may be modified so that the one or more size frequencies are input into the machine learning model and not the one or more sequence motif frequencies. As another example, process 5600 may include using only the motif frequencies of molecules (e.g., second column in FIG. 55). In this case, block 5640 and block 5660 are optional. Block 5670 may be modified so that the one or more sequence motif frequencies are input into the machine learning model and not the one or more size frequencies.

Process 5600 may include additional implementations, such as any single implementation or any combination of implementations described herein and/or in connection with one or more other processes described elsewhere herein.

Although FIG. 56 shows example blocks of process 5600, in some implementations, process 5600 may include additional blocks, fewer blocks, different blocks, or differently arranged blocks than those depicted in FIG. 56. Additionally, or alternatively, two or more of the blocks of process 5600 may be performed in parallel.

### VII. Enriching for Regions

The preference of DNA fragments associated with a certain epigenome status to exhibit a particular set of end motifs can be used to enrich a sample for DNA with that particular epigenome status. Accordingly, embodiments can enrich a sample for clinically-relevant DNA, including DNA from a particular tissue. For example, only DNA fragments having a particular ending sequence may be sequenced, amplified, and/or captured using an assay. As another example, filtering of sequence reads can be performed.

A. Physical Enrichment

Physical enrichment may be performed in various ways, e.g., via targeted sequencing or PCR, as may be performed using particular primers or adapters. If a particular end motif of an ending sequence is detected, then an adapter can be added to the end of the fragment. Then, when sequencing is performed, only DNA fragments with the adapter will be sequenced (or at least predominantly sequenced), thereby providing targeted sequencing.

As another example, primers that hybridize to the particular set of end motifs can be used. Then, sequencing or amplification can be performed using these primers. Capture probes corresponding to the particular end motifs can also be used to capture DNA molecules with those end motifs for further analysis. Some embodiments can ligate a short oligonucleotide to the end of a plasma DNA molecule. Then, a probe can be designed such that it would only recognize a sequence that is partially the end motif and partially the ligated oligonucleotide

Some embodiments can use CRISPR-based diagnostic technology, e.g. using a guide RNA to localize a site corresponding to a preferred end motif for the clinically-relevant DNA and then a nuclease to cut the DNA fragment, as may be done using Cas-9 or Cas-12. For example, an adapter can be used to recognize the end motif, and then CRISPR/Cas9 or Cas-12 can be used to cut the end motif/adaptor hybrid and create a universal recognizable end for further enrichment of the molecules with the desired ends.

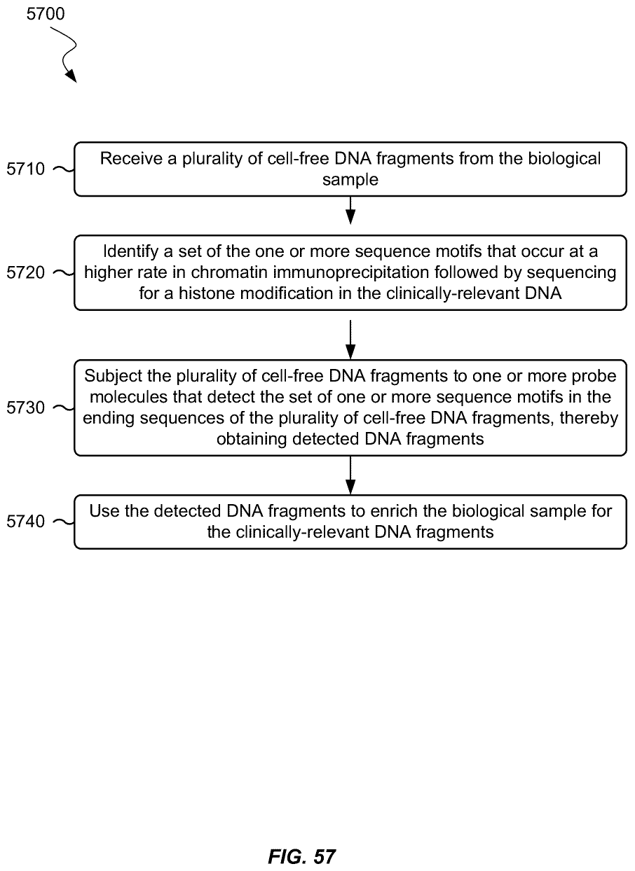

FIG. 57 is a flowchart of an example process 5700 associated with enriching a biological sample for clinically-relevant DNA. The biological sample may include clinically-relevant DNA and other DNA that are cell-free. In some implementations, one or more process blocks of FIG. 57 may be performed by a system (e.g., measurement system 5900). In some implementations, one or more process blocks of FIG. 57 may be performed by another device or a group of devices separate from or including the system. Additionally, or alternatively, one or more process blocks of FIG. 57 may be performed by one or more components of measurement system 5900, such as assay 5908, assay device 5910, detector 5920, logic system 5930, local memory 5935, external memory 5940, storage device 5945, and/or processor 5950. Process 5700 may include aspects described with process 1700.

At block 5710, a plurality of sequence reads of the cell-free DNA fragments is received. The plurality of sequence reads include ending sequences corresponding to ends of the plurality of cell-free DNA fragments. One or more sequence motifs may correspond to one or more ending sequences of each cell-free DNA fragment. Block 5710 may be performed in a similar manner as block 1710.

At block 5720, a set of the one or more sequence motifs is identified. The set of the one or more sequence motifs occur at a higher rate in chromatin immunoprecipitation followed by sequencing (ChIP-seq) for a histone modification in the clinically-relevant DNA than in sequencing without chromatin immunoprecipitation. Identifying the sequence motifs may be similar to the procedure described with process 1700 and with FIGS. 3, 10, 11, and 51.

At block 5730, the plurality of cell-free DNA fragments may be subjected to one or more probe molecules that detect the set of one or more sequence motifs in the ending sequences of the plurality of cell-free DNA fragments, thereby obtaining detected DNA fragments. Such use of probe molecules can result in obtaining detected DNA fragments. In one example, the one or more probe molecules can include one or more enzymes that interrogate the plurality of cell-free DNA fragments and that append a new sequence that is used to amplify the detected DNA fragments. In another example, the one or more probe molecules can be attached to a surface for detecting the sequence motifs in the ending sequences by hybridization.

At block 5740, the detected DNA fragments are used to enrich the biological sample for the clinically-relevant DNA fragments. In some embodiments, using the detected DNA fragments to enrich the biological sample may include amplifying the detected DNA fragments. In some embodiments, using the detected DNA fragments to enrich the biological sample for the clinically-relevant DNA fragments may include capturing the detected DNA fragments and discarding non-detected DNA fragments.

Process 5700 may further include analyzing the enriched biological sample to determine a tissue of origin or a classification of a level of a disease. Analyzing the enriched biological sample may include sequencing DNA fragments in the enriched biological sample.

Process 5700 may include additional implementations, such as any single implementation or any combination of implementations described herein and/or in connection with one or more other processes described herein.

Although FIG. 57 shows example blocks of process 5700, in some implementations, process 5700 may include additional blocks, fewer blocks, different blocks, or differently arranged blocks than those depicted in FIG. 57. Additionally, or alternatively, two or more of the blocks of process 5700 may be performed in parallel.

B. In Silico Enrichment

The in silico enrichment can use various criteria to select or discard certain DNA fragments. Such criteria can include end motifs, open chromatin regions, size, sequence variation, methylation and other epigenetic characteristics. Epigenetic characteristics include all modifications of the genome that do not involve a change in DNA sequence. The criteria can specify cutoffs, e.g., requiring certain properties, such as a particular size range, methylation metric above or below a certain amount, combination of methylation status of more than one CpG sites (e.g., a methylation haplotype (Guo et al, Nat Genet. 2017; 49: 635-42)), etc., or having a combined probability above a threshold. Such enrichment can also involve weighting DNA fragments based on such a probability.

As examples, the enriched sample can be used to classify a pathology (as described above), as well as to identify tumor or fetal mutations or for tag-counting for amplification/deletion detection of a chromosome or chromosomal region. For instance, if a particular end motif or a set of end motifs are associated with liver cancer (i.e., a higher relative frequency than for non-cancer or other cancers), then embodiments for performing cancer screening can weight such DNA fragments higher than DNA fragments not having this preferred one or this preferred set of end motifs.

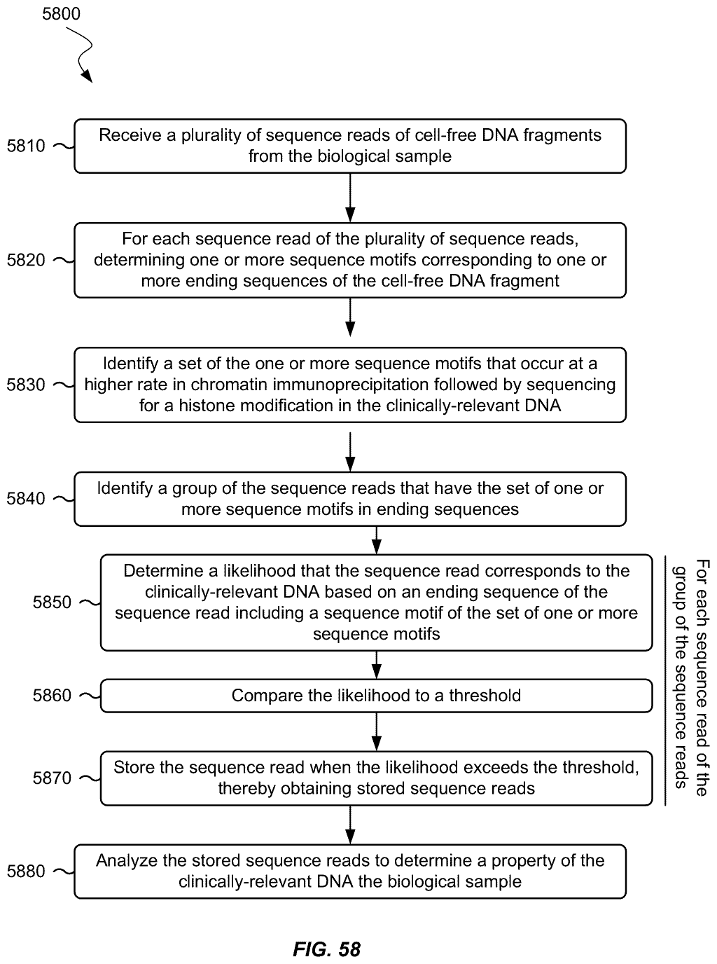

FIG. 58 is a flowchart of an example process 5800 associated with enriching a biological sample for clinically-relevant DNA. The biological sample may include clinically-relevant DNA and other DNA that are cell-free. The clinically-relevant DNA is DNA from a tissue of origin or DNA from a diseased tissue. In some implementations, one or more process blocks of FIG. 58 may be performed by a system (e.g., measurement system 5900). In some implementations, one or more process blocks of FIG. 58 may be performed by another device or a group of devices separate from or including the system. Additionally, or alternatively, one or more process blocks of FIG. 58 may be performed by one or more components of measurement system 5900, such as assay 5908, assay device 5910, detector 5920, logic system 5930, local memory 5935, external memory 5940, storage device 5945, and/or processor 5950. Process 5800 may include aspects described with process 1700.

At block 5810, a plurality of sequence reads of the cell-free DNA fragments is received. The plurality of sequence reads include ending sequences corresponding to ends of the plurality of cell-free DNA fragments. One or more sequence motifs may correspond to one or more ending sequences of each cell-free DNA fragment. Block 5810 may be performed in a similar manner as block 1710.

The plurality of sequence reads may be located in one or more predetermined genomic regions, wherein each of the one or more predetermined genomic regions has a histone modification associated with one or more target tissue types. The sequence reads may be aligned to a reference genome to determine their locations. The identification of sequence reads in these locations may be performed in a similar manner as block 1720.

At block 5820, one or more sequence motifs corresponding to one or more ending sequences of the cell-free DNA fragment are determined for each sequence read of a group of sequence reads. Block 5820 may be performed in a similar manner as block 1730.

At block 5830, a set of the one or more sequence motifs is identified. The set of the one or more sequence motifs occur at a higher rate in chromatin immunoprecipitation followed by sequencing (ChIP-seq) for a histone modification in the clinically-relevant DNA than in sequencing without chromatin immunoprecipitation. Identifying the sequence motifs may be similar to the procedure described with process 1700 and with FIGS. 3, 10, 11, and 51.

At block 5840, a group of the sequence reads that have the set of one or more sequence motifs in ending sequences is identified. This can be viewed as a first stage of filtering.

At block 5850, a likelihood that the sequence read corresponds to the clinically-relevant DNA based on an ending sequence of the sequence read including a sequence motif of the set of one or more sequence motifs is determined for each sequence read of the group of sequence reads. For instance, for each sequence read of the group of the sequence reads, a likelihood that the sequence read corresponds to the clinically-relevant DNA can be determined based on an ending sequence of the sequence read including a sequence motif of the set of one or more sequence motifs.

At block 5860, the likelihood is compared to a threshold for each sequence read of the group of sequence reads. As an example, the threshold can be determined empirically. For instance, various thresholds can be tested for samples that a concentration of the clinically-relevant DNA can be measured for a group of sequence reads. An optimal threshold can maximize the concentration while maintaining a certain percentage of the total number of sequence reads. The threshold could be determined by one or more given percentiles (5^{th}, 10^{th}, 90^{th}, or 95^{th}) of the concentrations of one or more end motifs present in the healthy controls or in control groups exposed to similar etiological risk factors but without diseases. The threshold could be a regression or probabilistic score.

At block 5870, the sequence read is stored when the likelihood exceeds the threshold for each sequence read of the group of sequence reads. The sequence read can be stored in memory (e.g., in a file, table, or other data structure), thereby obtaining stored sequence reads. Sequence reads having a likelihood below the threshold can be discarded or not stored in the memory location of the reads that are kept, or a field of a database can include a flag indicating the read had a lower threshold so that later analysis can exclude such reads. As examples, the likelihood can be determined using various techniques, such as odds ratio, z-scores, or probability distributions.

At block 5880, the stored sequence reads are analyzed to determine a property of the clinically-relevant DNA the biological sample. For example, the property may be any described herein, including with other flowcharts. For instance, the property of the clinically-relevant DNA the biological sample can be a fractional concentration of the clinically-relevant DNA. As another example, the property can be a level of pathology of a subject from whom the biological sample was obtained, where the level of pathology is associated with the clinically-relevant DNA. As another example, the property can be a gestational age of a fetus of a pregnant female from whom the biological sample was obtained.

Other criteria can be used to determine the likelihood. Sizes of the plurality of cell-free DNA fragments can be measured using the sequence reads. The likelihood that a particular sequence read corresponds to the clinically-relevant DNA can be further based on a size of the cell-free DNA fragment corresponding to the particular sequence read.

Methylation can also be used. Thus, embodiments can measure one or more methylation statuses at one or more sites of a cell-free DNA fragment corresponding to a particular sequence read. The likelihood that the particular sequence read corresponds to the clinically-relevant DNA can be further based on the one or more methylation statuses. As a further example, whether a read is within an identified set of open chromatin regions can be used as a filter.

Process 5800 may include additional implementations, such as any single implementation or any combination of implementations described herein and/or in connection with one or more other processes described herein.

Although FIG. 58 shows example blocks of process 5800, in some implementations, process 5800 may include additional blocks, fewer blocks, different blocks, or differently arranged blocks than those depicted in FIG. 58. Additionally, or alternatively, two or more of the blocks of process 5800 may be performed in parallel.

### VIII. Example Systems

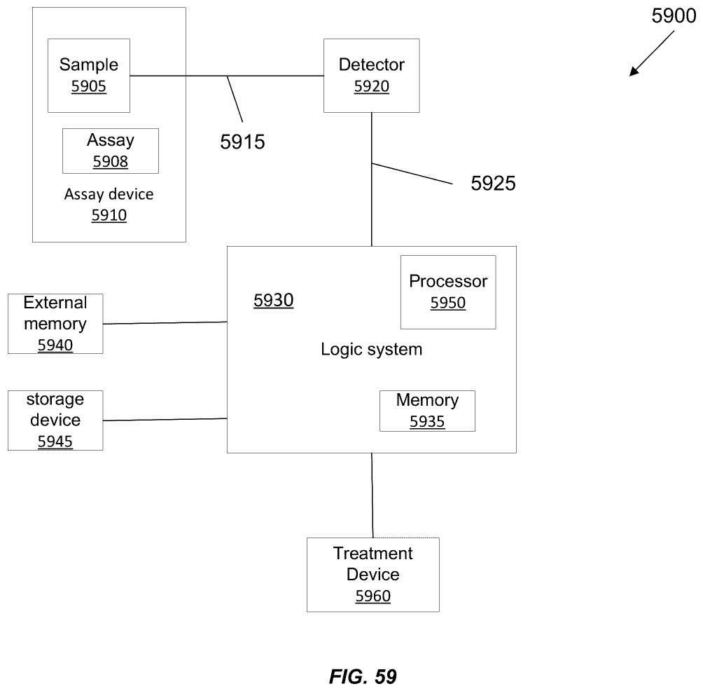

FIG. 59 illustrates a measurement system 5900 according to an embodiment of the present disclosure. The system as shown includes a sample 5905, such as cell-free nucleic acid molecules (e.g., DNA and/or RNA) within an assay device 5910, where an assay 5908 can be performed on sample 5905. For example, sample 5905 can be contacted with reagents of assay 5908 to provide a signal of a physical characteristic 5915 (e.g., sequence information of a cell-free nucleic acid molecule). An example of an assay device can be a flow cell that includes probes and/or primers of an assay or a tube through which a droplet moves (with the droplet including the assay). Physical characteristic 5915 (e.g., a fluorescence intensity, a voltage, or a current), from the sample is detected by detector 5920. Detector 5920 can take a measurement at intervals (e.g., periodic intervals) to obtain data points that make up a data signal. In one embodiment, an analog-to-digital converter converts an analog signal from the detector into digital form at a plurality of times.

Assay device 5910 and detector 5920 can form an assay system, e.g., a sequencing system that performs sequencing according to embodiments described herein. A data signal 5925 is sent from detector 5920 to logic system 5930. As an example, data signal 5925 can be used to determine sequences and/or locations in a reference genome of nucleic acid molecules (e.g., DNA and/or RNA). Data signal 5925 can include various measurements made at a same time, e.g., different colors of fluorescent dyes or different electrical signals for different molecule of sample 5905, and thus data signal 5925 can correspond to multiple signals. Data signal 5925 may be stored in a local memory 5935, an external memory 5940, or a storage device 5945. The assay system can be comprised of multiple assay devices and detectors.

Logic system 5930 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 5930 and the other components may be part of a stand-alone or network connected computer system, or they may be directly attached to or incorporated in a device (e.g., a sequencing device) that includes detector 5920 and/or assay device 5910. Logic system 5930 may also include software that executes in a processor 5950. Logic system 5930 may include a computer readable medium storing instructions for controlling measurement system 5900 to perform any of the methods described herein. For example, logic system 5930 can provide commands to a system that includes assay device 5910 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 5900 may also include a treatment device 5960, which can provide a treatment to the subject. Treatment device 5960 can determine a treatment and/or be used to perform a treatment. Examples of such treatment can include surgery, radiation therapy, chemotherapy, immunotherapy, targeted therapy, hormone therapy, and stem cell transplant. Logic system 5930 may be connected to treatment device 5960, e.g., to provide results of a method described herein. The treatment device may receive inputs from other devices, such as an imaging device and user inputs (e.g., to control the treatment, such as controls over a robotic system).

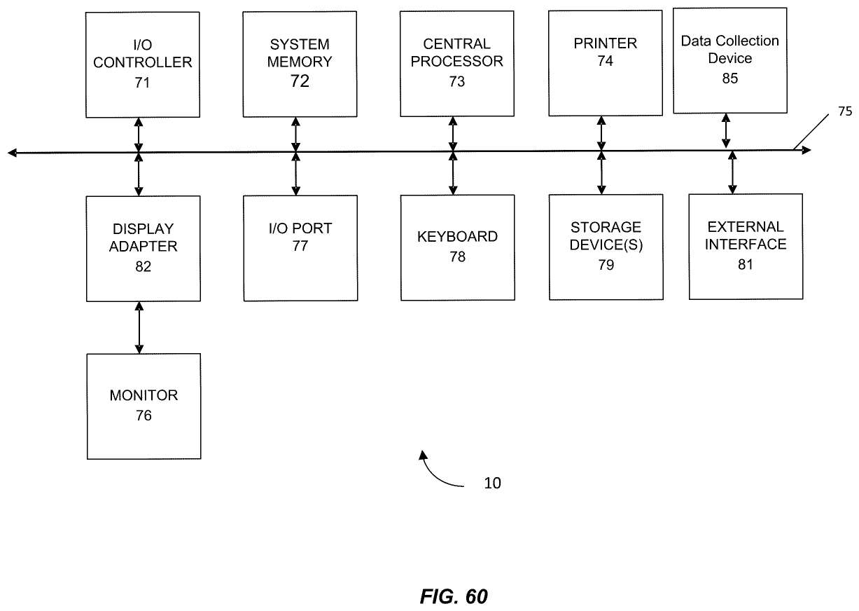

Any of the computer systems mentioned herein may utilize any suitable number of subsystems. Examples of such subsystems are shown in FIG. 60 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 60 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, Lightning, Thunderbolt). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely” “only”, and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

1. A method of analyzing a biological sample, the biological sample including cell-free DNA fragments, the method comprising:
receiving a plurality of sequence reads of the cell-free DNA fragments;
identifying a group of sequence reads located in one or more genomic regions, wherein each of the one or more genomic regions has a histone modification associated with a target tissue type;
determining a value of a fragmentomic feature of each cell-free DNA fragment corresponding to each sequence read in the group of sequence reads;
determining one or more relative frequencies of cell-free DNA fragments having values of the fragmentomic feature in a set of one or more value ranges, wherein the set of the one or more value ranges occurs at a differential rate in chromatin immunoprecipitation followed by sequencing for the histone modification associated with the one or more genomic regions than in sequencing without chromatin immunoprecipitation;
determining an aggregate value of the one or more relative frequencies;
comparing the aggregate value to one or more calibration values; and
determining an amount of the histone modification in the biological sample using the comparison.

2. The method of claim 1, wherein:
the fragmentomic feature is a sequence motif corresponding to an ending sequence of an end of the cell-free DNA fragment, and
the one or more value ranges are one or more sequence motifs.

3. The method of claim 1, wherein:
the fragmentomic feature is a size, and
the one or more value ranges are one or more size ranges.

4. The method of claim 1, wherein:
the fragmentomic feature is a topological form, and
the one or more value ranges are one or more topological forms.

5. The method of claim 1, wherein:
the fragmentomic feature is a nucleosomal footprint, and
the one or more value ranges are one or more nucleosomal footprints.

6. The method of claim 1, further comprising:
comparing the amount of the histone modification to one or more second calibration values, and:
using the comparison of the amount of the histone modification to the one or more second calibration values, either:
determining a fractional concentration of the target tissue type,
determining a classification of a level of a disorder, or
determining a classification of a transplant status of the target tissue type.

7. A method of analyzing a biological sample, the biological sample including cell-free DNA fragments, the method comprising:
receiving a plurality of sequence reads of the cell-free DNA fragments, wherein the plurality of sequence reads include ending sequences corresponding to ends of the cell-free DNA fragments;
identifying a group of sequence reads located in one or more genomic regions, wherein each of the one or more genomic regions has a histone modification associated with a target tissue type;
for each sequence read of the group of sequence reads, determining one or more sequence motifs corresponding to one or more ending sequences of a corresponding cell-free DNA fragment;
determining one or more relative frequencies of a set of the one or more sequence motifs, wherein the set of the one or more sequence motifs occurs at a higher rate in chromatin immunoprecipitation followed by sequencing for the histone modification associated with the one or more genomic regions than in sequencing without chromatin immunoprecipitation;
determining an aggregate value of the one or more relative frequencies;
comparing the aggregate value to one or more calibration values; and
determining a fractional concentration of cell-free DNA fragments from the target tissue type using the comparison, wherein the one or more calibration values are determined from one or more calibration samples whose fractional concentrations of cell-free DNA fragments from the target tissue type are known.

8. The method of claim 7, wherein:
the one or more relative frequencies are one or more first relative frequencies,
the aggregate value is a first aggregate value, and
the one or more calibration values are determined by:
for each calibration sample of one or more calibration samples:
determining one or more second relative frequencies of the set of the one or more sequence motifs in the one or more genomic regions, and
determining a second aggregate value of the one or more second relative frequencies,
thereby associating each of one or more second aggregate values with known fractional concentrations, wherein the one or more calibration values include the one or more second aggregate values.

9. The method of claim 7, wherein the aggregate value is a value selected from a group consisting of: (i) an entropy value; (ii) a sum of relative frequencies; (iii) a ratio of relative frequencies; and (iv) a multidimensional data point corresponding to a vector of counts for the set of the one or more sequence motifs.

10. The method of claim 7, wherein a sequence motif of the set of the one or more sequence motifs corresponds to a single nucleotide, a two-nucleotide sequence, a three-nucleotide sequence, a four-nucleotide sequence, a five-nucleotide sequence, a six-nucleotide sequence, or a seven-nucleotide sequence.

11. The method of claim 10, wherein the sequence motif includes the nucleotide at the end of the cell-free DNA fragment.

12. The method of claim 10, wherein the sequence motif is at the 5′ end.

13. The method of claim 7, wherein the target tissue type comprises the placenta, liver, heart, neutrophils, monocytes, B cells, adipose, or NK cells.

14. The method of claim 7, wherein the target tissue type is the placenta,
the method further comprising:
determining a classification of a pregnancy-associated disorder or a gestational age using the fractional concentration.

15. The method of claim 7, further comprising determining a classification of a level of cancer using the fractional concentration.

16. The method of claim 7, wherein:
the group of sequence reads is a first group of sequence reads,
the one or more genomic regions are one or more first genomic regions,
the histone modification is a first histone modification,
the target tissue type is a first target tissue type,
the set of the one or more sequence motifs is a set of one or more first sequence motifs,
the one or more relative frequencies are one or more first relative frequencies,
the aggregate value is a first aggregate value,
the one or more calibration samples are one or more first calibration samples, and
the fractional concentration is a first fractional concentration,
the method further comprising:
identifying a second group of sequence reads located in one or more second genomic regions, wherein each of the one or more second genomic regions have a second histone modification associated with a second target tissue type,
for each sequence read of the second group of sequence reads, determining one or more second sequence motifs corresponding to the one or more ending sequences of a corresponding cell-free DNA fragment,
determining one or more second relative frequencies of a set of the one or more second sequence motifs, wherein the set of the one or more second sequence motifs occurs at a higher rate in chromatin immunoprecipitation followed by sequencing for the second histone modification associated with the one or more second genomic regions than in sequencing without chromatin immunoprecipitation,
determining a second aggregate value of the one or more second relative frequencies,
comparing the second aggregate value to one or more second calibration values, and
determining a second fractional concentration of cell-free DNA fragments from the second target tissue type using the comparison, wherein the one or more second calibration values are determined from one or more second calibration samples whose fractional concentrations of DNA fragments from the second target tissue type are known.

17. A method of analyzing a biological sample, the biological sample including cell-free DNA fragments, the method comprising:
receiving a plurality of sequence reads of the cell-free DNA fragments, wherein the plurality of sequence reads include ending sequences corresponding to ends of the cell-free DNA fragments;
identifying a group of sequence reads located in one or more genomic regions, wherein each of the one or more genomic regions have a histone modification associated with a target tissue type;
for each sequence read of the group of sequence reads, determining one or more sequence motifs corresponding to one or more ending sequences of a corresponding cell-free DNA fragment;
determining one or more relative frequencies of a set of the one or more sequence motifs, wherein the set of the one or more sequence motifs occurs at a higher rate in chromatin immunoprecipitation followed by sequencing for the histone modification associated with the one or more genomic regions than in sequencing without chromatin immunoprecipitation;
determining an aggregate value of the one or more relative frequencies;
comparing the aggregate value to one or more calibration values; and
estimating a first value for a characteristic of the target tissue type using the comparison, wherein the one or more calibration values are determined from one or more calibration samples whose values for the characteristic of the target tissue type are known.

18. The method of claim 17, wherein:
the one or more relative frequencies are one or more first relative frequencies,
the aggregate value is a first aggregate value, and
the one or more calibration values are determined by:
for each calibration sample of one or more calibration samples:
determining a second relative frequency of the set of the one or more sequence motifs in the one or more genomic regions, and
determining a second aggregate value of the one or more second relative frequencies,
thereby associating each of one or more second aggregate values with known values for the characteristic, wherein the one or more calibration values include the one or more second aggregate values.

19. (canceled)

20. The method of claim 17, wherein the target tissue type is an organ that has cancer.

21-25. (canceled)

26. The method of claim 17, wherein:
the aggregate value is a first aggregate value, and
the one or more calibration values are one or more first calibration values,
the method further comprising:
measuring sizes of the cell-free DNA fragments using the sequence reads, determining one or more size frequencies of the sequence reads for one or more size ranges,
determining a second aggregate value of the one or more size frequencies, and
comparing the second aggregate value to one or more second calibration values,
wherein estimating the first value for the characteristic comprises using the comparison of the second aggregate value to the one or more second calibration values, wherein the one or more second calibration values are determined from the one or more calibration samples.

27-87. (canceled)
